(19) United States
(12) Patent Application Publication
(10) Pub. No.: US 2025/0377301 A1
(43) Pub. Date: Dec. 11, 2025

(54) STABILISED FLUOROPHORES, COMPOSITIONS, METHODS OF PREPARATION, CONJUGATES THEREOF, AND METHODS OF USE

(71) Applicant: Freistaat Bayern (Ludwig-Maximilians-Universität München), München (DE)

(72) Inventors: Oliver THORN-SESHOLD, München (DE); Viktorija GLEMBOCKYTE, München (DE); Benedikt BAUMGARTNER, Haunscheim (DE); Alexander WIEGAND, München (DE)

(21) Appl. No.: 18/737,536

(22) Filed: Jun. 7, 2024

(51) Int. Cl. G01N 21/64; C07C 251/20; C07D 487/14; C07D 498/14; C07F 5/02; C07F 7/08; C07F 9/6527; C07F 9/6561; G01N 33/533

(52) U.S. Cl. CPC G01N 21/6456; C07C 251/20; C07D 487/14; C07D 498/14; C07F 5/022; C07F 7/0814; C07F 9/6527; C07F 9/6561; G01N 33/533; C07C 2603/24

(57) ABSTRACT

The present application relates to photostabilized or “self-healing” dyes that are constructed by attaching an azoaryl unit to a parent dye such that its optical properties are improved, particularly its optical properties that are most relevant in highly-demanding imaging, including instant brightness, signal stability, photon budget, and rate of detecting localisations. Therefore the compounds of the invention can be useful for high-performance imaging, e.g. delivering higher spatial and temporal resolution, within a shorter experimental acquisition time, and with more confidence, whether in oxygen-free conditions or in aerated conditions.

### TECHNICAL FIELD

The present application relates to compounds containing a fluorophore unit and an azoaryl unit that modifies the fluorescence properties of the fluorophore, which have one of the formulas (I.1 to I.9), and more particularly, to such compounds wherein the triplet excited state of the compound is nonradiatively depopulated faster than is the triplet excited state of the respective fluorophore F. Particularly in imaging applications where light is applied at high intensities, such compounds display advantageous performance features, such as increased photon budget, increased instant brightness, and/or increased survival time, relative to comparable fluorophore moieties without an azoaryl moiety. They are thus suitable for fluorescent imaging in a variety of situations and applications.

### BACKGROUND OF THE INVENTION

Fluorescent dyes are crucial for a vast range of applications in biology, physics, chemistry, medicine, biotechnology, and other scientific and technical fields. For example, fluorescence microscopy is an unparalleled technique to image structures on the sub-micron level, and dynamics on the millisecond scale, with high sensitivity and selectivity. The performance features of a fluorescent dye substantially determine the applications it can be used in, and the power or value of the results that can be obtained.

Particularly for highly-demanding imaging applications where fluorescent dyes are subjected to high excitation intensities, three key features that should be maximised for best imaging performance are (a) the photon budget, i.e. how many fluorescence photons on average are emitted before the dye is destroyed during imaging; (b) the instant brightness, i.e. on average how many fluorescence photons are emitted per unit of time; and, related to both of these, (c) the survival time, i.e. for how long on average that the fluorescent dye can be imaged under a specific condition before it is destroyed. Also, (d) the tendency of the dye to photosensitise surrounding molecules should usually be minimised, to avoid damaging effects or phototoxicity in a sample.

The requirements for fluorescent dyes with high performance in demanding imaging applications, and the logic of photoprotective additives, have been reviewed or treated elsewhere (see e.g. (Pati; Proceedings of the National Academy of Sciences 2020 117, 24305-24315); (Zhang; Angewandte Chemie International Edition 2022 61, e202112959); and references therein). A brief overview is given in the following.

The four aforementioned features (a)-(d) are all strongly impacted by the tendency of the photoexcited dye to enter a triplet state by intersystem crossing, and by the average lifetime of that triplet state before it recovers back to a singlet state. Triplet states do not fluoresce, so they are also known as “dark states”; the more time that a dye spends in a triplet dark state, the less time that it can undergo productive excitation-fluorescence cycles. Therefore (b) the instant brightness of a dye can be improved by either reducing the tendency to enter the triplet state (the intersystem crossing yield, Φ_{ISC}), or by reducing the average lifetime before a triplet state recovers to a singlet state (the triplet lifetime, T₃). Also, triplet states can undergo several types of reactivity that damage the dye or molecules in its environment; therefore, minimising Φ_{ISC} and/or T₃ are helpful to improve (a) photon budget and (c) survival time, while also reducing (d) photosensitisation and phototoxicity.

There are several reasons why minimising T₃ is particularly needed for good performance in the many “highly-demanding” imaging applications which use high excitation intensity (photons per unit surface area per unit time), e.g. in some super-resolution imaging techniques such as STED. For example, (1) even dyes that have Φ_{ISC} values that are considered to be very low will eventually enter a dark triplet state. The higher the excitation intensity applied, the more potential cycles of excitation-fluorescence will be missed during a given dark state lifetime.

Therefore, in demanding applications, a dye's T₃ value can strongly limit its performance even if it has a low Φ_{ISC}, since a maximum limit for its possible fluorescence output is an average instantaneous brightness of (1/Φ_{ISC}) photons per time T₃. Typical values for a good fluorophore operating in oxygen-free conditions without other additives might be Φ_{ISC}˜0.01% and T₃ 100 μs; but if T₃ could be reduced to e.g. 100 μs, the maximum instant brightness of the fluorophore under high-intensity illumination could be improved by a factor of up to one million. Maximising the instant brightness is important for high performance: high instant brightness means that more imaging frames can be acquired per second (allowing higher temporal resolution imaging, and/or faster completion of an experiment); and high instant brightness also makes it more likely that a sufficient number of photons to pass a given detection threshold can be captured before a motile fluorescent object leaves a detected area (allowing better object tracking). (2) Another consideration of super-resolution methods is that the spatial resolution obtained improves if more photons can be collected: so, since minimising T₃ improves the photon budget, it directly impacts the spatial resolution too.

Additives that are variously known as e.g. “photostabilisers”, “photoprotective agents”, “triplet state quenchers”, etc can be added to samples as “solution state additives” with the aim that they should reduce the lifetime of a fluorophore's dark states and/or increase its photon budget, for example by accelerating the recovery of a fluorophore's undesired triplet state to the singlet ground state, by an intermolecular reaction (Rasnik; Nature Methods 2006 3, 891-893). Such additives include e.g. Trolox, cyclooctatetraene (COT), 1,4-diazabicyclo[2.2.2]octane (DABCO), or their derivatives. Such additives face many practical problems. Typically, very high concentrations are needed for efficient photoprotection (e.g. mM range), since the additive must react with the dye's triplet state before other species do; and these high concentrations may be toxic to live biological samples, disruptive to biological or physical structures, and/or difficult to establish due to poor solubility or partitioning of the additive in the medium used. Also, specifically regarding COT and its derivatives, which are currently popular triplet state quencher additives, they are not efficient at depopulating triplet excited states of all fluorophores, but only of a few.

A few groups have explored the properties of fluorescent dyes which are constructed by covalently tethering triplet state quenchers to fluorophores (or scaffolding them to be in close proximity to each other, using a DNA template); such dyes are referred to as “self-healing” dyes. These were intended to allow photoprotection without applying high concentrations of solution-state additives, and to achieve fluorescence performance that should be independent of the concentration of the dye. This concept was pioneered by Liphardt et al in the 1980s (Liphardt; Optics Communications 1981 38, 207-210), reporting E1a and E1b, i.e. a stilbene as a triplet state quencher, that was covalently connected to a fluorophore although not in electronic conjugation to it. For clarity, throughout this application, “conjugation” of motifs is intended in the biochemical sense (covalent connection but not electronic conjugation), unless “electronic conjugation” is explicitly specified.

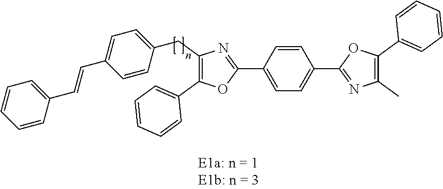

This concept of self-healing dyes was next significantly progressed two decades later when the groups of Cordes, Tinnefeld, and Blanchard created and studied further examples of fluorophores covalently connected to photoprotective motifs. These works showed that quenching the fluorophore triplet state is indeed the major mechanism of photostabilisation at work (van der Velde; ChemPhysChem 2013 14, 4084-4093), which is why most of the literature and the present invention focus on triplet state quenching.

Blanchard et al. have been active studying fluorophores with tethered COT derivatives such as E2a and E2b ((Zheng; J. Phys. Chem. Lett. 2012 3, 2200-2203); (Pati; Proceedings of the National Academy of Sciences 2020 117, 24305-24315); and references therein). Also, in two patent families Blanchard et al. have described fluorophores that are covalently tethered to photoprotective agents that belong to other molecular classes such as nitroaryls or chromanols (includes Trolox), e.g. E3a and E3b, as well as thiols and phenols ((Blanchard; WO2013109859A1); (Blanchard; WO2010096720A2); (Zheng; J. Phys. Chem. Lett. 2012 3, 2200-2203)). Azoaryls or azobenzenes were not taught or suggested as potential photoprotective motifs. This accurately reflects that azoaryls have never been considered for this purpose in small molecule fluorescence imaging.

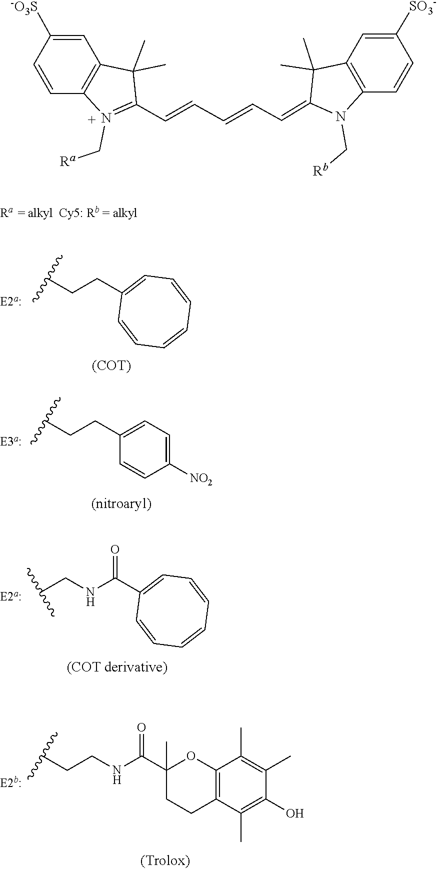

Cordes, Tinnefeld and coworkers have further elucidated the performance-enhancing mechanisms and performance-limiting challenges of self-healing dyes. A summary article from 2020 sets out the state of the art, notably giving a list of fluorophores (rhodamines, cyanines, carbopyronines, bophy, oxazines, fluoresceins) and photostabilizer types (COT, Trolox [TX], nitroaryls [e.g. NPA/NBA], nickel complex [e.g. TrisNTA], stilbene, phenol [e.g. BHT]) that have been combined for self-healing dyes (FIG. 5 in (Isselstein; J. Phys. Chem. Lett. 2020 11, 4462-4480)). From this review we note that the limited efficiency of intramolecular triplet quenching in self-healing dyes remains a major, performance-limiting challenge: since their “improvement [of photostability, signal stability, and brightness] could, however, not reach that acquired from intermolecular stabilization of the native fluorophore [by solution state additives]”. We note particularly that “to date, self-healing dyes were shown to be rather ineffective in the presence of molecular oxygen . . . the large discrepancy between the performance of self-healing dyes with and without oxygen is not yet solved . . . [one cause] might be that oxygen is always faster compared to triplet-quenching via intramolecular stabilizers . . . [but, quenching] rates [for COT as a stabiliser] are comparable to those estimated with molecular oxygen, suggesting a more complex reason than only kinetics for the experimental findings of rather unstable fluorophores in the presence of oxygen even when using COT.”

Therefore, there remain unsolved challenges for self-healing small molecule dyes, including to: (i) rationally design covalently-tethered triplet-quenching moieties such that the rate of fluorophore triplet energy transfer to the quencher is maximally fast, which would help to avoid damaging reactivity between the fluorophore triplet and other molecules in samples, such as molecular oxygen which is consistently present in live biological samples. However, we identify an additional challenge, which we have not seen being discussed in the relevant literature: (ii) once the quencher moiety has accepted the triplet energy, it becomes a triplet state. The quencher moiety has its own triplet state lifetime, before it undergoes intersystem crossing back to the singlet manifold; and during this lifetime it can also react with molecules in its surroundings to cause damage by chemically destroying the quencher moiety (which leaves the fluorophore moiety unprotected, removing all performance benefits of the self-healing construction), or by directly or indirectly chemically destroying the fluorophore (for example, indirectly via converting molecular oxygen to reactive singlet oxygen that then reacts with the fluorophore), and/or by directly or indirectly reacting with its surroundings (which can negatively impact imaging performance if e.g. the chemical features of a molecule that are required in order to bind the self-healing dye are destroyed, such that no more dye will dock at that binding site, meaning that no more imaging can take place).

Therefore, to maximise the performance of a self-healing dye, we conclude that the quencher moiety should be designed and integrated in the dye such that not only (i) its rate of accepting triplet energy from the fluorophore moiety is as fast as possible, but also (ii) its triplet state lifetime is as short as possible. In this context, we note that although the most widely-used triplet quencher moiety COT has a good triplet accepting rate, its triplet lifetime of ca. 100 μs (Das; J. Chem. Soc., Faraday Trans. 1994 90, 963-968) is not short but is similar to typical organic molecules. A principle inventive aspect of this work has been to discover an improved chemical moiety that does fill both criteria (i) and (ii), which to our knowledge has never yet been applied to provide a self-healing small molecule fluorophore. The present application confirms experimentally that the claimed compounds can be used to create self-healing dyes from a range of valuable fluorophore moieties. This chemical moiety in the present invention is an azoaryl moiety, i.e. a derivative or a heterocyclic analogue of azobenzene. In the present application “aryl” is defined as an aromatic or heteroaromatic moiety having 5 to 12 ring atoms selected from C, N, O and S. Examples thereof include phenyl, naphthalene, pyridine, N-pyridinium, pyrimidine, imidazole, pyrazole, thiazole, thiophene, benzothiazole, triazole, and tetrazole or any combination of the same. The aryl group can be optionally substituted, e.g. by alkyl, halogen, ester, amide, nitrile, nitro, trifluoromethyl, alkoxy, amine, hydroxy, carboxylic acid, sulfonic acid, phosphonic acid, sulfonamide, carboxamide, and carboxylester or any combination of the same.

Azoaryl moieties will be described and specified in detail later, but at this stage it is useful to consider their general structural scope and features, as illustrated by azoaryl moieties AZ-1 through AZ-5 below. As shown, in principle, an azoaryl motif is one that contains two aryl groups (Aryl¹ and Aryl²) that are connected by an —N═N— bond, and where these two aryl rings are not connected to each other in an additional way that allows further electronic conjugation between them (i.e. not connected by ring fusion, nor by a biaryl bond (illustrated in the non-azoaryl compound NA-1), nor by e.g. a —CH═CH— linker, etc). The two aryl groups are not particularly limited. Both may be phenyl (AZ-2); or, one may be phenyl and the other a heteroaromatic ring (e.g. AZ-4); or, both may be heteroaromatic rings (e.g. AZ-5); and the aryl groups may optionally by connected to each other by a linker that does not allow electronic conjugation (e.g. AZ-3; typical linker backbones have 3 atoms (as in AZ-3) or 2 atoms). Azoaryls may be either E or Z configured at the central N═N double bond and may easily and reversibly be interconverted between E and Z isomers e.g. after photon absorption or single electron redox; for the entirety of this patent and its claims, any representation of an azoaryl should be understood to cover the pure E isomer, the pure Z isomer, or any mixture of the two. Any or all positions on the aryl rings may be substituted (e.g. with -alkyl instead of —H groups).

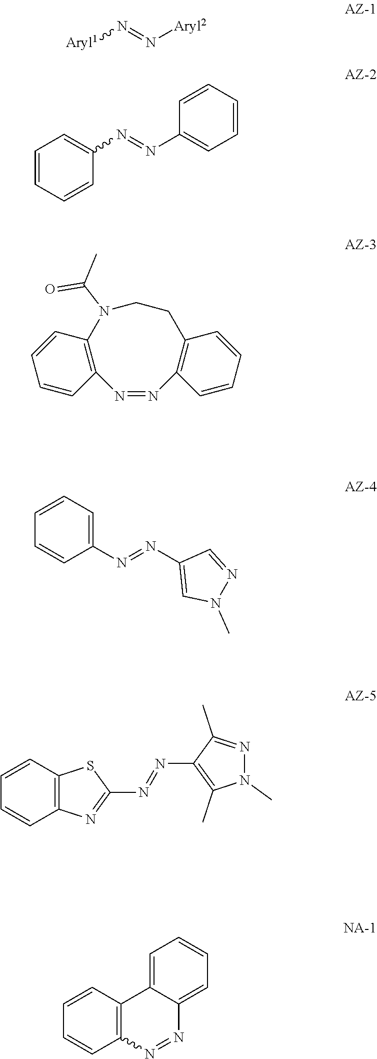

Azoaryls have never been shown as triplet-quencher moieties for self-healing small molecule fluorophores and one of the novelties of the present invention is the discovery that they can be used for this purpose. Azoaryls have been known for over 150 years, but have almost exclusively been studied and used in the context of their singlet-manifold photochemistry, as colorants (light absorption), as photoswitches (light absorption followed by E/Z isomerisation) (Jerca; Nature Reviews Chemistry 2022 6, 51-69)), and as fluorescence quencher moieties in FRET probes (they absorb energy from a nearby fluorophore moiety's singlet excited state, therefore suppressing fluorescence signal). In brief, it seems that the scientific literature has so overwhelmingly taught that azoaryls are highly effective fluorescence Quenchers, that apparently no prior research has been directed towards using them as enhancers of small molecule fluorescence.

Small molecule triplet sensitiser chromophores can perform triplet energy transfer (TET) to azobenzenes ((Monti; Journal of Photochemistry 1983 23, 249-256); (Bortolus; J. Phys. Chem. 1979 83, 648-652)) and the rates of TET to azobenzene can be exceptionally fast ((Monti; Chemical Physics Letters 1981 77, 115-119); (Jones; J. Am. Chem. Soc. 1965 87, 4219-4220)). We noted that this is encouraging for solving the challenge point (i) stated above, which had apparently not been perceived before, probably because the research in this prior art around TET in the context of azoaryls has been exclusively focused on the effects of TET on the azobenzene, and had not considered any potential utility for the performance of the small molecule chromophore. In brief, the known utility of TET on the azobenzene is that, since azobenzene T1 states collapse to give mainly the E-isomer of the singlet groundstate SO state, triplet-sensitised Z→E isomerisation of the azobenzene can be used to ensure highly complete net photoisomerisation of azobenzene derivatives, i.e. by irradiating a triplet sensitiser chromophore that is in the vicinity of an azobenzene. Such highly complete photo-triggered isomerisation is a much-pursued goal in the field of molecular photoswitching; and therefore it is unsurprising that developments in this area have focused on using small molecule chromophores that are very good triplet sensitisers (high Φ_{ISC}) such as heavy metal complexes, and phototoxic species such as methylene blue and eosin ((Isokuortti; Chem. Sci. 2021 12, 7504-7509); (Ronayette; Can. J. Chem. 1974 52, 1858-1867)), with the aim of delivering high sensitisation efficiency. We note that since high-Φ_{ISC} molecules perform poorly in fluorescence imaging, especially at high intensity (see above), it is unsurprising that none of these studies had considered or measured the fluorescence performance of the sensitiser molecule; i.e., this prior art has not taught towards the current invention which relies on optimising fluorescence properties. We note also that all of these studies were conducted in an intermolecular mode (no tether between sensitiser and azoaryl), i.e. none of this prior art was oriented towards the necessary tethered chemical design that is required for a self-healing dye (see above).

Azobenzene T1 states have an exceptionally short triplet lifetime before spontaneously decaying, ca. 10 μs, due to large spin-orbit coupling (Cembran; J. Am. Chem. Soc. 2004 126, 3234-3243)). As far as we are aware, this lifetime has not been exploited before in a small molecule context relevant to the current invention.

There is extensive prior art from the field of FRET which discourages the possibility of using azoaryls in high-performance fluorescent dyes. There are vast numbers of research reports and patents using azoaryl moieties that are covalently tethered to fluorophore moieties because the azoaryl quenches the fluorescence of the construct by FRET; these are accompanied by many commercially available azoaryl fluorescence quencher reagents (including DABCYL, the “BHQ” series of quenchers, BBQ-650, IQ4, and Eclipse), and such FRET quenching designs are significantly applied industrially (see (Chevalier; Chemistry—An Asian Journal 2017 12, 2008-2028); (Fang; Angewandte Chemie International Edition 2022 61, e202207188); and references therein). Not only is the performance aim the opposite of the current invention, but none of these reports have imaged the fluorescence of the tethered dye and found it to actually be better fluorescent than the isolated fluorophore. As a typical example, a FRET probe constructed as IRDye8000W-polypeptide-BHQ-3 (Linder; Bioconjugate Chem. 2011 22, 1987-1297) that was intended as a protease-cleavable NIR fluorogenic probe for matrix metalloproteinases, had more than 98% loss (quenching) of fluorescence intensity despite only having very little overlap of the absorption of the azoaryl BHQ-3 with the fluorophore IRDye8000W. Therefore, this large body of prior art around FRET applications essentially teaches that azoaryls would, if anything, suppress the fluorescence of covalently attached fluorophore moieties, i.e. it teaches directly away from the present invention, which will show how azoaryls can instead be used to significantly enhance selected fluorescence properties of nearby fluorophore moieties.

In 2021, Cordes published a study on the fluorescence properties of the fluorescent protein GFP that had been covalently tethered to known triplet quenchers (COT, trolox, and nitrophenyl) or to an azobenzene photoswitch (FIG. 2A in (Henrikus; ChemBioChem 2021 22, 3283-3291)). GFP is a barrel-structured protein, where the fluorescent chromophore sits inside the beta-barrel, protected from collisions with surrounding molecules, but the attachment site for the moieties used is on the outside of the barrel. Therefore the moieties cannot collide with the chromophore; and since triplet energy transfer requires spatial overlap of orbitals (i.e. collision of the moieties), it was unsurprising that “none of these [COT, trolox, and nitrophenyl, known] photostabilisers increased or decreased the photobleaching time, count-rate, total photon count and SNR strongly”. It was stated that the azobenzene-tethered-GFP exhibited some increased photostability, with most fluorescence performance metrics being changed by between −5% up to +50%), but the mechanism for this result was not triplet-state quenching nor was it claimed to be triplet quenching: indeed, the noisy fluorescence blinking patterns (FIG. 6B in (Henrikus; ChemBioChem 2021 22, 3283-3291)) are very different to the stable fluorescence emission pattern of self-healing fluorophores (e.g. traces such as FIG. 4c in (Isselstein; J. Phys. Chem. Lett. 2020 11, 4462-4480), which is a paper authored by the same group). (1) This result therefore directly teaches away from the idea that an azoaryl could succeed in improving fluorescence properties by triplet state quenching in self-healing dyes. We note several further aspects of this prior art that either situate it in a separate area from the current invention, or else teach away from the current invention: (2) This prior art report is about a protein fluorophore, whereas the current invention concerns small molecules. The challenges for photostabilisation, and the methods of solving them, are very different between shielded protein fluorophores where collisional mechanisms are blocked (hence the motivation for using protein fluorophores in biology—their signal is not affected by their environment), and small molecule fluorophores where collisional mechanisms are crucial and must be addressed. (3) This prior art shows that there is no photostabilisation of the GFP by the azobenzene when oxygen is present, which teaches away from the performance obtainable with the current invention.

Against this background of prior art that either does not give precedents for, or else actively teaches away from the invention, we discovered that azoaryl moieties can act as extremely powerful triplet-quenching photostabilisers for small molecule fluorophore moieties when covalently tethered together, in such a way that the fluorescence of the resulting self-healing dye is drastically increased (in several examples with >1000% enhancement depending on the evaluation metrics). The current invention of azoaryl-based self-healing dyes will therefore have utility in delivering high-performance imaging outcomes in highly-demanding settings especially where these involve high intensity excitation and/or a need for high photon acquisition count.

### SUMMARY OF THE INVENTION

The invention describes fluorophore-containing compositions wherein a fluorophore unit is in proximity to one or more azoaryl units that increase the rate of relaxation from the dark triplet state of the fluorophore to the ground state of the fluorophore, so reducing the time a fluorophore unit spends in dark states, so modifying its photophysical characteristics, such as by improving the total photon budget emitted from a single fluorophore before photobleaching and increasing the instantaneous brightness of a fluorophore. The molecule of the invention can optionally contain a tether unit for labelling target species.

The present invention can be applied to biological, biophysical, or molecular imaging, where high illumination intensities are needed for high-spatiotemporal-resolution measurements and where unwanted dark states and photobleaching of fluorophores often limits the overall time and signal-to-noise ratio of the measurement; as well as to super-resolution imaging, which benefits from high photon budget, signal stability, dye survival time; to PCR, sequencing and microarray applications.

The invention is summarized in the appended claims.

### DESCRIPTION OF THE FIGURES

FIG. 1: FIG. 1a shows the normalised autocorrelation functions G(T) for unstabilised EY-P (data as indicated with the labelled arrow, fitted with the dashed line), and self-healing EY-AK (data as indicated with the labelled arrow, fitted with the dotted line), both acquired at 50 μW in solution measurements. FIG. 1b shows normalised G(T) for self-healing EY-AK recorded at varying excitation powers. FIG. 1c shows triplet fractions for EY-P calculated from fitted FCS data at varying excitation powers. FIG. 1d shows autocorrelation analysis of fluorescence intensities measured on surface immobilized unstabilised Atto542 and self-healing Atto542-AO. FIG. 1e shows single-molecule trajectory results acquired at 50 ms/frame time resolution for self-healing and parent fluorophores.

FIG. 2: FIG. 2a-b show how exchangable 8 nt DNA imager strands bearing parent fluorophores or self-healing compounds of the invention were used in DNA-PAINT imaging, under exclusion of molecular oxygen. FIG. 2c-g show DNA-PAINT imaging with AlexaFluor647 and with its analogous self-healing compounds of the invention. The compounds of the invention have substantially increased imaging quality (FIG. 2c-e), total photon budget (FIG. 2f), and localisation frequency (FIG. 2g), compared to the parent fluorophore. Scale bars in FIG. 2c,d,e are 500 nm.

FIG. 3: FIG. 3a-d show example trajectories and total photon count statistics for single molecule imaging of Cy5 in deoxygenated samples optionally in the presence of azobenzene PST-2S (2 mM) freely diffusing in the solution as a photoprotective agent. FIG. 3e-f show a schematic of DNA-PAINT imaging and data extraction. FIG. 3g-j show results of DNA-PAINT imaging with exchangeable 8 nt DNA imager strands bearing Cy5 or AbberiorStar635P, optionally in the presence of an azoaryl-bearing “photostabiliser” 21 nt strand that binds permanently onto the DNA docking site, wherein the dye on the imager strand and the azoaryl on the stabiliser strand are brought in proximity when both are hybridised to the scaffold strand. Reference measurements were performed under normal (oxygenated) conditions; measurements in the presence of the azoaryl photostabiliser were performed under deoxygenated conditions. The image brightness is enhanced by the azoaryl strand (FIG. 2g,i); and the rate of detecting localisations, as well as the bright time of each localization are increased many-fold (FIG. 3h,j), which indicate that the azoaryl can take over the role of triplet quenching that is usually performed by molecular oxygen and improve the photostability

FIG. 4: FIG. 4 illustrates the advantages which are achieved with Cy5-DABCYL and further compounds of the invention.

### DETAILED DESCRIPTION OF THE INVENTION

### Definitions

Unless stated otherwise the following definitions apply.

The term “alkyl” refers to a saturated or unsaturated, linear or branched or cyclic hydrocarbon moiety containing between 1 to 50 carbon atoms and optionally 0 to 19 heteroatoms, preferably 0 to 10 heteroatoms, wherein the heteroatoms are typically chosen from O, N, S, Se, Si, Hal, B or P, preferably chosen from O, N. The term “alkylene” is used to specifically indicate a bivalent alkyl moiety.

An aliphatic moiety is a saturated or unsaturated, linear or branched or cyclic moiety containing between 1 to 50 carbon atoms and optionally 0 to 19 heteroatoms, preferably 0 to 15 heteroatoms, wherein the heteroatoms are typically chosen from O, N, S, Se, Si, Hal, B or P, preferably chosen from O, N, Hal, S, Si or P, more preferably O, N, Hal, S, Si, even more preferably chosen from O or N.

Halogen (or halide or -Hal) refers to —F, —Cl, —Br or —I, preferably —F or —Cl.

If a moiety is referred to as being “optionally substituted” by one or more substituents it can in each instance include one or more of the indicated substituents, chosen the same or different.

Fluorophore units in the compounds of the invention may be fluorescent diagnostic agents, used to label or detect or image target biological species or structures, as explained below.

The term “acceptable salt” refers to a salt of a compound of the present invention. Suitable acceptable salts include acid addition salts which may, for example, be formed by mixing a solution of compounds of the present invention with a solution of an acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compound carries an acidic moiety, suitable acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of acceptable salts include, but are not limited to, counterions listed in Berge; J. Pharm. Sci. 1977 66, 1-19.

The term “acceptable ester” refers to an ester of a compound of the present invention. Suitable acceptable esters include acetyl, butyryl, and iso-butyryl esters, and acetoxymethyl ethers.

### Components and Structure of the Compound of the Invention

The invention concerns compounds containing a fluorophore unit, an azoaryl unit, and optionally but advantageously a tether unit, wherein the units may be connected to each other via linker units, optionally making use of functional groups referred to as connection points to join these units and/or linkers to each other. These five unit types will be explained first, before explaining the overall structure of the compounds of the invention.

Fluorophore Unit: The fluorophore unit contains a fluorophore to be photostabilised. The fluorophore unit is preferably a small molecule fluorophore. In one aspect, the fluorophore contains a conjugated π-system that involves from 4 to 50 carbon atoms, optionally containing 1 or more heteroatoms, preferably 1 to 20 heteroatoms, more preferably 1 to 10 heteroatoms, wherein the heteroatoms can be independently selected from O, N, S, Se, Si, Hal, B or P, preferably independently selected from O, N, Hal, S, Si or P, more preferably independently selected from O, N, Hal, S, Si, even more preferably independently selected from O, N or Hal; and where the fluorophore unit has an overall molecular weight of <2000 Da. The fluorophore is not particularly limited, but may in principle be any fluorescent species that is employed for purposes that include detecting its fluorescence signal, and where it is desired to enhance its fluorescence signal as described; preferably, the fluorophore can be chosen from any class of fluorophores known to those skilled in the art (non-limiting examples of fluorophore units preferable for use in compounds of the invention can be found in literature, e.g. (Grimm; Nature Methods 2022 19, 149-158) and references therein); preferably, the fluorophore unit can be chosen as a fluorophore which is already useful for highly-demanding imaging applications, so that its performance can be further improved by modifying it into a self-healing dye according to the invention; preferably, the fluorophore is an organic fluorophore, which can be charged or uncharged, and the chemical structure of which can include polycyclic, aromatic, conjugated polyunsaturated, and/or heteroaromatic motifs that are the chromophores responsible for absorption of excitation light and emission of fluorescence light, or can contain a latent form of such chromophores as is the case with spirocyclised lactones of fluoresceins and rhodamines, or with O- or N-acylated versions of fluorophores that rely on free phenol or aniline electron donating groups for fluorescence emission, as is known to those skilled in the art. The fluorophore is not a genetically-encoded fluorescent protein such as GFP protein.

Fluorescence emission may occur with different patterns in time and/or space and/or with respect to reactions or environment. For example, fluorophores may be continuously fluorescent; or they may intrinsically “blink” spontaneously (switch between bright emissive and dark non-emissive states) e.g. through a reversible intramolecular cyclisation reaction; or they may blink extrinsically during assay conditions as a result of reactions with additives such as thiols during imaging; or they may exhibit environment-dependent fluorescence; or their fluorescence may turn on following a covalent reaction including a photochemical reaction or enzymatic reaction, or following a non-covalent association including intercalation between DNA bases or binding to nucleic acids in the minor groove, or following complexation such as of a metal cation e.g. calcium. Explicitly, these patterns of fluorescence emission have utility for different purposes, and for all of them it can be of great value to improve the fluorescence emission characteristics as discussed above (higher photon budget, higher instantaneous brightness, lower generation of singlet oxygen, greater fluorophore resistance to photobleaching, etc) for the emissive or “bright times” of the fluorophore (e.g. for an intrinsically blinking fluorophore such as hmSiR, during times when the fluorophore is fully conjugated as the xanthene form).

Optionally, the fluorophore may be chosen to be a widely-used fluorophore; or to be a derivative of the same with an identical π-system-chromophore (for example, 4-(N-methyl)amino-7-isobutylcoumarin as a derivative whose π-system-chromophore is identical to that of 4-(N-methyl)amino-7-methylcoumarin); or a derivative with a near-identical r-system-chromophore in the sense that important auxochromic groups are modified without changing the atoms and groups which define the π-system-chromophore (for example, 4-(N-methyl)amino-7-isobutylcoumarin as a derivative whose important auxochromic amine substituent is a modification of that used in 4-(1-azetidinyl)-7-methylcoumarin but whose π-system-chromophore is otherwise identical).

Optionally, the fluorophore may be chosen from any of several series of structurally and/or functionally related fluorophores, some of which are shown in Scheme 1, and including but not limited to:
the cyanine (“Cy”) series of polymethine fluorophores, a general structure of which is represented in Scheme 1, and which includes structurally related Cy3, Cy3.5, Cy3B, Cy5, AlexaFluor 555, AlexaFluor 647, Cy5.5, Cy7, Cy7.5, FNIR-tag, ICG, IRDye 8000W, SNIR1, IRDye 700, IRDye 78, DiO, Dil, DiO, DiR, and their derivatives, and polymethine analogues such as MeOFlav7 and JuloFlav7;
the coumarin series of fluorophores and their auxochromically related species, including 4-methylumbelliferone, AlexaFluor 350, AlexaFluor430, pacific blue, Star 440 SXP, Atto 425, and Coumarin 153;
the BODIPY series of fluorophores and their auxochromically related species, including BODIPY FL, BODIPY 507/545, BODIPY TR, BDP R6G, BODIPY 558/568, BDP 581/591, BOPHY, C11-BODIPY, and BDP 650/665;
the xanthene series of fluorophores, which includes the fluorescein, rhodamine, and rhodol series of fluorophores and their auxochromically related species; a general structure of xanthene-based fluorophores is represented in Scheme 1 and clarifies that xanthene analogues where the bridge atom is carbon (e.g. carbofluoresceins, carborhodamines, carborhodols (also known as fluorescein-carbopyronine hybrids), and ketorhodamines, including ATTO 647N, JF585), silicon (e.g. silarhodamines, including HMSiR, HM-DS655, SiP650, SiR 700, 680SiR, SiR680, HMSiR_{indol}, HMSiRⱼᵤₗₒₗ, HMSiR_{THQ}, or Yale676_{sb}), or other atoms including phosphorus (phosphorhodamines, including Nebraska Red 700), nitrogen, boron, sulfur, or selenium, are included; and that variants that are lactonisable to the nonfluorescent spiro form or permanently in the open form or which are spontaneously blinking (e.g. hydroxymethyl or hydroxyethyl xanthenes such as HMSiR, HEtetTFER, Yale676sb, HMSiR_{THQ}) are also included. Other examples therefore include calcein, Fluo-4, fluoresceins such as fluorescein, oregon green, tetrachlorofluorescein, rhodols such as rhodol, Nebraska Red rhodol, rhodamines (e.g. Rhodamine 110, Rhodamine 6G, Rhodamine B, tetramethylrhodamine (TMR or TAMRA), JF526, Alexa Fluor 488, Alexa Fluor 546, Alexa Fluor 594, DyLight488, JF549, Sulforhodamine 101, AlexaFluor 532, JFX549, Atto 647, Atto 488, CF 488, Rhodamine 800, Atto 550, HEtetTFER, HMJF526, HMRG, HMAcRG, JF593, sulfone-rhodamine), and xanthene analogues without a pendant aryl ring (e.g. pyronines/carbopyronines such as Atto 520, JF585, Atto 610, CP550, SiP650, TMDHS), and expanded xanthene fluorophores such as naphthofluoresceins, as well as auxochromically related species where the π-system is affected by cross-conjugated auxochromic groups, such as in AlexaFluor 568, Atto 594, and AlexaFluor 633;
the phenoxazine series of fluorophores and related species, including Atto 655, Atto 680, resorufin, brilliant cresyl blue, benzophenoxazines such as Nile blue and Nile red, and related species such as the thionines methylene blue, azure B, and azure A;
and many other series of widely-used fluorophores, including squaraines, Hoechst series fluorophores such as Hoechst 33342, pyrene series fluorophores such as AlexaFluor 405 and Cascade Blue, and metal-complexing fluorogenic fluorophores such as benzofuran series fluorophores including Fura-1 and Fura-2;
and derivatives of the same, such as calcein AM or Fura-2-acetoxymethyl ester or Fluo-4 AM (derivatives of calcein or Fura-2 or Fluo-4, respectively).

For all fluorophore classes, their variants with substitution patterns that tune physicochemical properties e.g. solubility or biolocalisation are included (e.g. bearing one or more sulfonates, phosphonates, halides, alkyl groups, etc), as are commercial fluorophores with such structures (e.g. AlexaFluor594 and Texas Red are understood as included within the xanthene class).

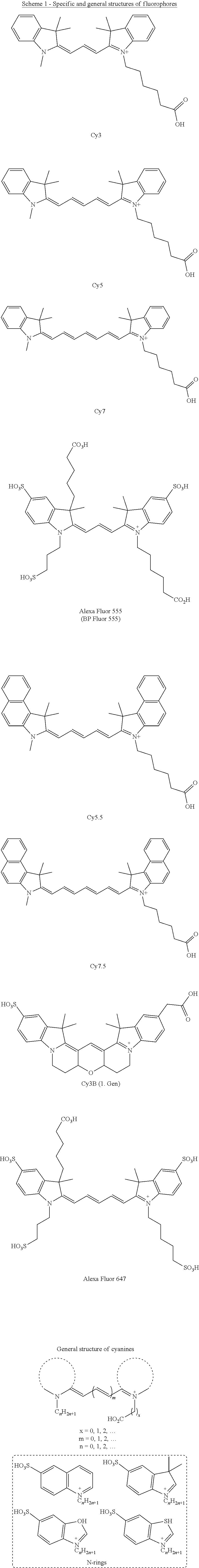

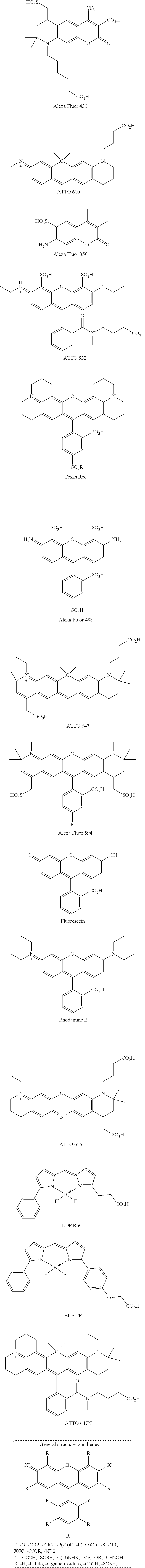

Connection Points: As shown in Scheme 1, the fluorophores in the fluorophore unit can feature one or more connection points suitable for covalently linking them with the azoaryl unit and optionally the tether unit. Most simply, such a connection point can be a carboxylic acid group (see Cy3, Cy5, Cy7, Cy7.5, AlexaFluor555, Cy5.5, etc) which can be used to form a covalent link such as an amide, for example, by reacting an activated carboxylic acid derivative (of the fluorophore, such as an N-hydroxysuccinimide (NHS) ester), with a suitable partner reactive group (such as an aliphatic primary or secondary amine) that is borne by e.g. a linker-azoaryl unit-linker-tether unit. Other suitable connection points include but are not limited to aliphatic or aromatic primary or secondary amines e.g. for forming amide connections, alkynes such as —C≡CH e.g. for forming triazole connections, maleimides e.g. for forming thioether connections, azides such as —N₃ e.g. for forming triazole or amide connections, etc. Derivatives of a fluorophore can be made by methods known to those skilled in the art, such that a new connection point is introduced to it, e.g. by reacting a fluorophore or fluorophore derivative with a bifunctional linker. One example of this is 2-O-alkylation of a rhodol with tert-butyl 2-bromoacetate, followed by ester hydrolysis (i.e. the product is a 2-OCH₂CO₂H rhodol derivative) to introduce a carboxylic acid connection point. Many other connection point types, and methods for their introduction, are known to those skilled in the art, and further examples are given below as they occur.

Azoaryl unit: an azoaryl unit is defined to contain one, two, three, or four azoaryl species (aryl-N═N-aryl′) that may be the same or different. An azoaryl species may in principle have any aryl and aryl′ groups freely chosen, the same or different, as defined below, including where zero, one or both aryl rings are not carboaromatic rings (e.g., phenyl rings) but instead are heteroaromatic rings.

Examples of azoaryl species include but are not limited to those shown in Schemes 2-3 and described in the following. They may be unsubstituted except at their connection point(s) (e.g. azobenzenes M1, M3); or they and/or their connection point(s) may be substituted with one or more electron-withdrawing groups (e.g. halogen (e.g., M9), ester, amide, nitrile, nitro, trifluoromethyl) or one or more electroneutral groups (e.g. alkyl) or one or more electron-donating groups (e.g. alkoxy (e.g., M8), amine, hydroxy) or any combination of the same. Further substituents include carboxylic acid, sulfonic acid, phosphonic acid, sulfonamide, carboxamide, and carboxylester.

The azoaryl species can be annelated in that the two aryl rings are connected by both the diazene (—N═N—) and by a separate aliphatic linkage containing between 1-12 carbon atoms and between 1-8 heteroatoms chosen from N, O, F, Si, P, S, and Cl; preferably the annelating linkage is (—CH₂—)₁₋₃, or derivatives thereof in which 0-2 of the carbon atoms in the bridge are replaced by heteroatoms chosen from N, O, Si, P, and S, and wherein the annelating linker may be further substituted with small motifs; this includes e.g. dibenzooxadiazepine (one heavy atom in the annelating linkage) (e.g., M11), diazocine (two), diazonine (three), and their derivatives. Any aryl-N═N-aryl′ species may feature both aryl and aryl′ rings as phenyl rings (e.g., M1-M10) or naphthalene, or one of them (e.g., M12, M14, M17), or neither of them (e.g., M13, M15, M16, M18), whereby the non-phenyl ring or rings are heteroaromatic rings that may be e.g. pyridine, N-pyridinium, pyrimidine, imidazole, pyrazole, thiazole, thiophene, benzothiazole, triazole, tetrazole, or any combination of the same.

Optionally, where the azoaryl is used monovalently (single connection point to the rest of the photostabilised construct), it may be chosen from azoaryls such as shown in Scheme 2, or their congeners that are derived by minor modifications.

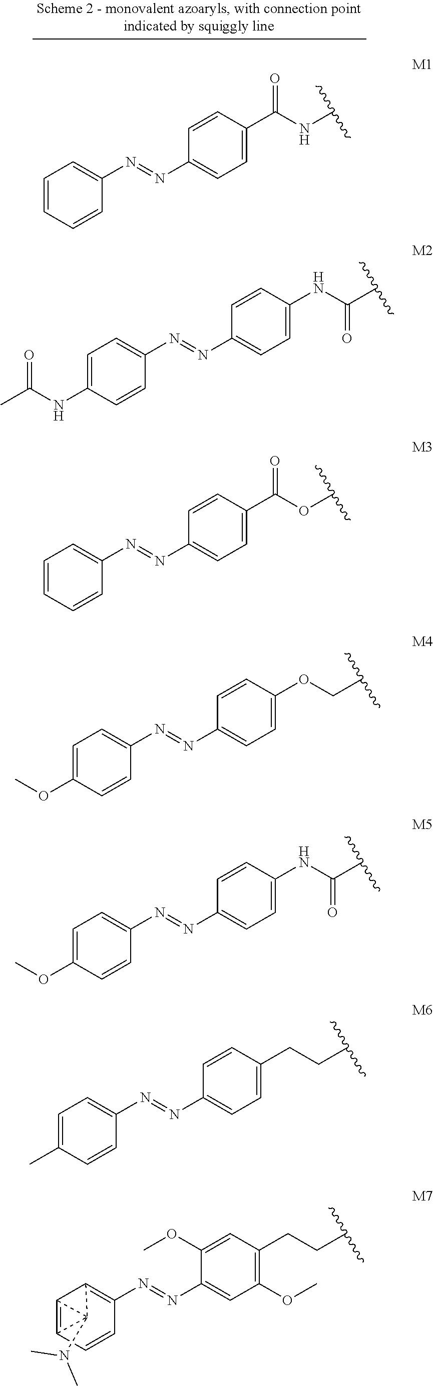

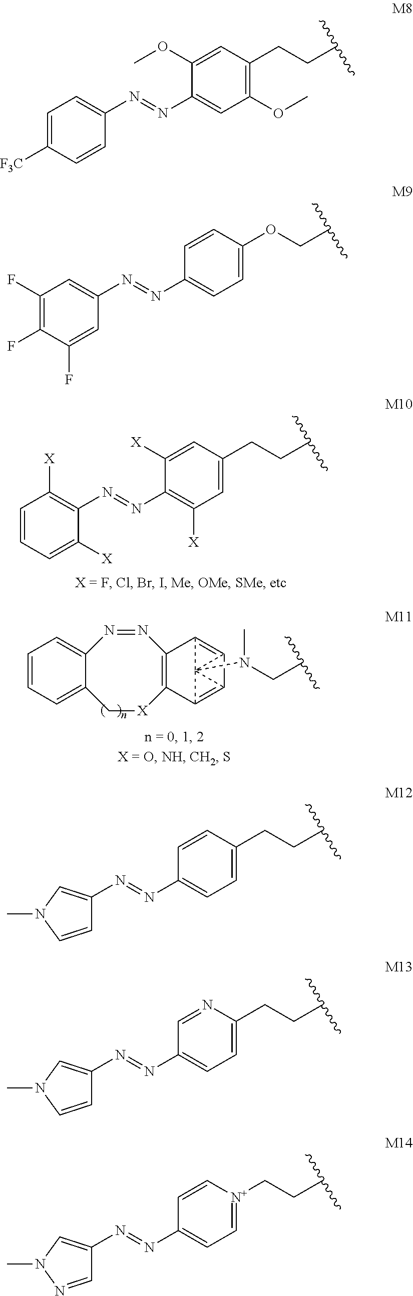

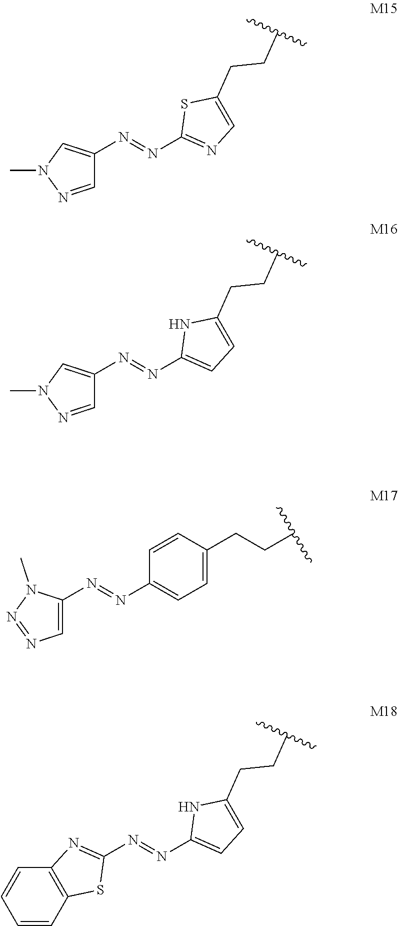 [SMILES: *CCc1cnc(/N=N/c2cnn(C)c2)s1 ; *CCc1ccc(/N=N/c2cnn(C)c2)[nH]1 ; *CCc1ccc(/N=N/c2cnnn2C)cc1 ; *CCc1ccc(/N=N/c2nc3ccccc3s2)[nH]1]

Optionally, where the azoaryl is used bivalently (two connection points to the rest of the photostabilised construct), it may be chosen from azoaryls such as B1-B8 in Scheme 3, or their congeners that are derived by minor modifications.

Optionally, where the azoaryl is used trivalently (three connection points to the rest of the photostabilised construct), it may be chosen from azoaryls such as E3 shown in Scheme 3.

An azoaryl unit may contain one (as in M1-M17, B1-B9), or it may contain two, three, or four, aryl-N═N-aryl′-type diazene motifs (“multiple azoaryl”). The diazene motifs in a multiple azoaryl may be electronically non-conjugated with each other, e.g. as in the dimeric (Y1) and tetrameric (D1) examples in Scheme 3; or the diazenes may be electronically conjugated to each other, as in bis-azobenzenes C1-C3 (monovalent) or E1-E2 (bivalent) or E3 (trivalent); or any combination of the same.

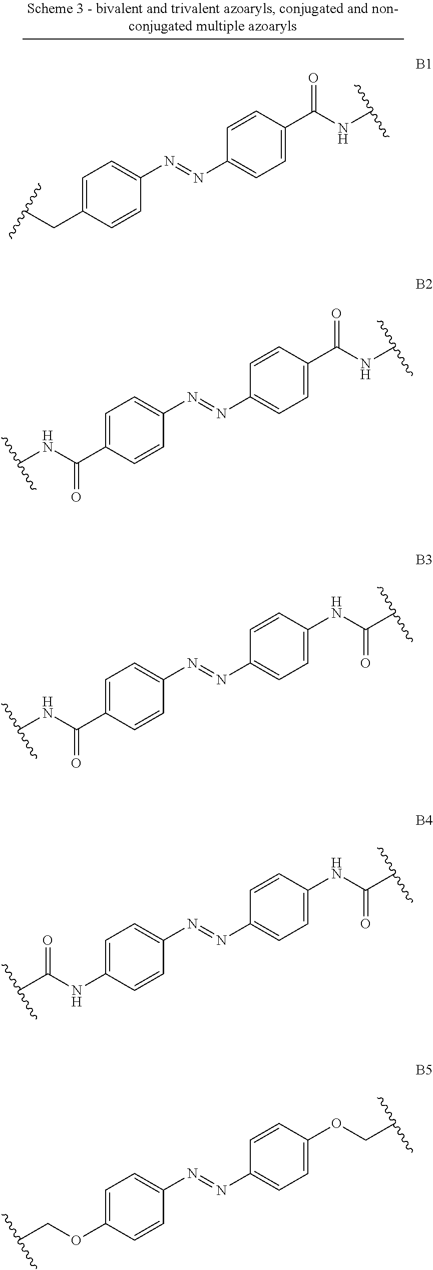 [SMILES: *Cc1ccc(/N=N/c2ccc(C(=O)N*)cc2)cc1 ; *NC(=O)c1ccc(/N=N/c2ccc(C(=O)N*)cc2)cc1 ; *NC(=O)c1ccc(/N=N/c2ccc(NC(*)=O)cc2)cc1 ; *C(=O)Nc1ccc(/N=N/c2ccc(NC(*)=O)cc2)cc1 ; *COc1ccc(/N=N/c2ccc(OC*)cc2)cc1]

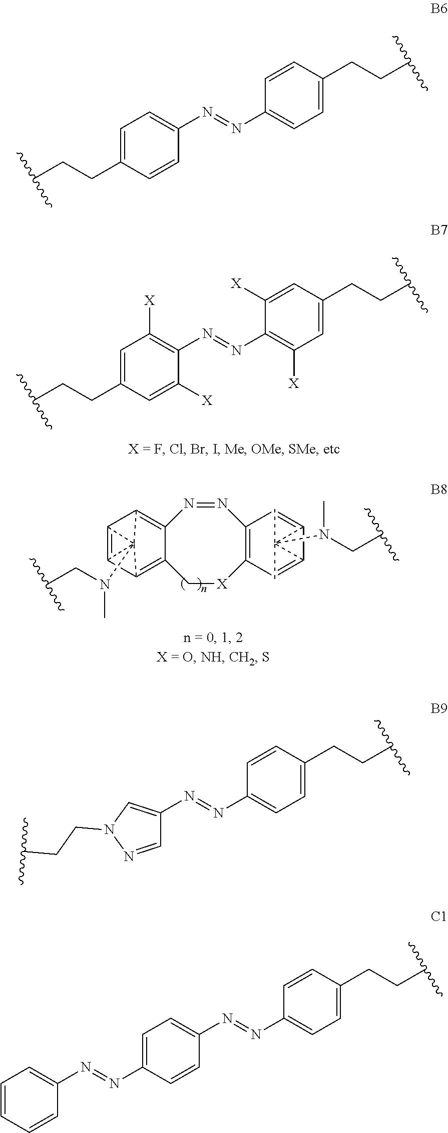

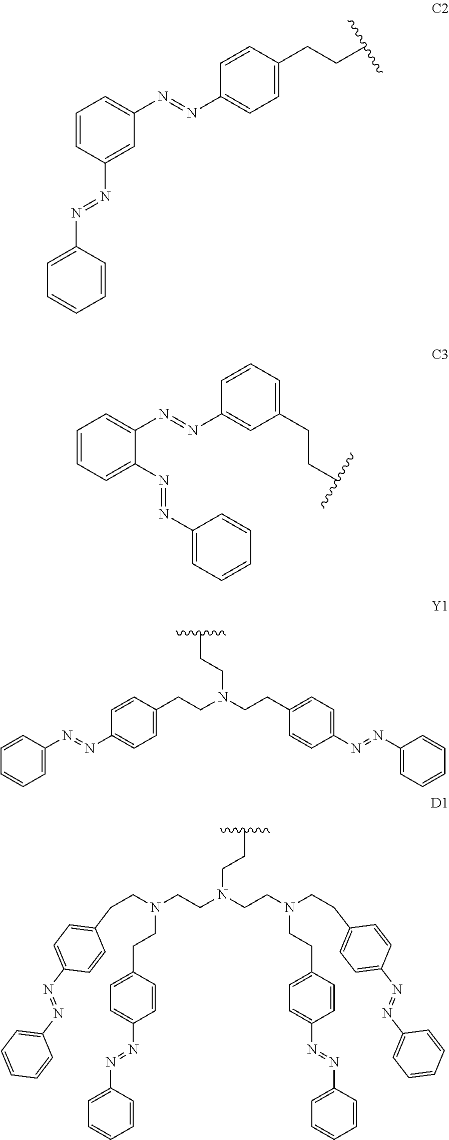 [SMILES: *CCc1ccc(/N=N/c2cccc(/N=N/c3ccccc3)c2)cc1 ; *CCc1cccc(/N=N/c2ccccc2/N=N/c2ccccc2)c1 ; *CCN(CCc1ccc(/N=N/c2ccccc2)cc1)CCc1ccc(/N=N/c2ccccc2)cc1 ; *CCN(CCN(CCc1ccc(/N=N/c2ccccc2)cc1)CCc1ccc(/N=N/c2ccccc2)cc1)CCN(CCc1ccc(/N=N/c2ccccc2)cc1)CCc1ccc(/N=N/c2ccccc2)cc1]

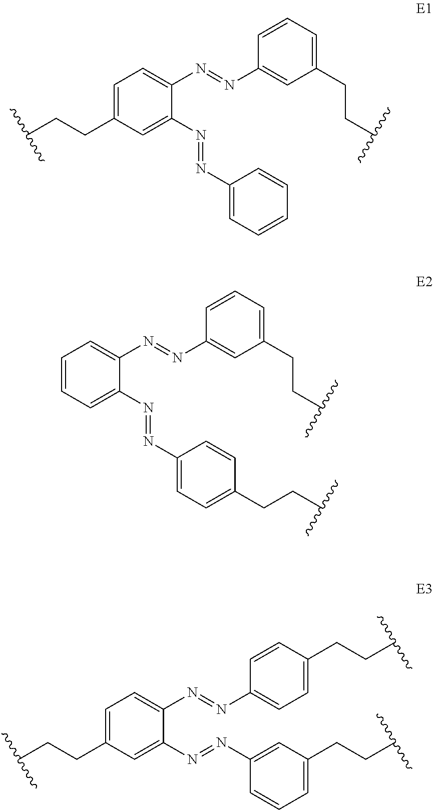 [SMILES: *CCc1cccc(/N=N/c2ccc(CC*)cc2/N=N/c2ccccc2)c1 ; *CCc1ccc(/N=N/c2ccccc2/N=N/c2cccc(CC*)c2)cc1 ; *CCc1ccc(/N=N/c2ccc(CC*)cc2/N=N/c2cccc(CC*)c2)cc1]

The same variety of connection points introduced above can be used to attach an azoaryl unit to other units of the conjugate of the invention. With reference to the examples shown above, these include but are not limited to: carboxylic acid (can form e.g. benzamides as in M1, B2; or esters as in M3); aryl amine (can form e.g. anilides M2, M5, B4, M5); alkyl group (M6, M7, M8, B6, B7, C2, C3); or aryl alcohol (can form e.g. ethers M4, M9, B5, or, esters); or for multivalent azoaryls, any combination of the same.

Tether Unit: A tether unit is defined as a molecular motif which allows covalently attaching, reversible-covalently attaching, or noncovalently associating its appended fluorophore-azoaryl conjugate onto a target species or structure of interest. The target can be freely chosen for the purposes of imaging it or modifying its fluorescence or other properties; examples include but are not limited to: a protein e.g. a protein or glycoprotein of interest in biology, optionally in living cells or on a virus; or a DNA strand, e.g. a short oligonucleotide strand for DNA-PAINT imaging; or an antibody; or a glycan or glycoprotein; and in all cases, the target can then for example be imaged with the high-performance fluorescence that the invention allows.

The aim for a covalent (or reversible-covalent) tether unit is typically to achieve high chemical specificity and/or high rate of the labelling reaction, often within a complex or biological environment. Therefore, such tether units can advantageously be chosen by those skilled in the art, including but not limited to: substrates for self-labelling protein tags (e.g. SNAP, CLIP, and Halo tag substrates in T1-T3 in Scheme 4), bioorthogonally reactive moieties (e.g. alkynes or strained alkynes (e.g., T5, T7-T10), e.g. for click reactions with azides; azides (e.g., T6), e.g. for click reactions with alkenes or alkynes or for Staudinger-Bertozzi ligation to phosphines; phosphines (e.g., T11) for Staudinger-Bertozzi ligation to azides; tetrazines or strained alkenes (e.g., T12-T15) e.g. for inverse electron demand Diels Alder (IEDDA) reaction with strained alkenes or tetrazines respectively); or chemical groups with favourable reactivity of labelling: such as isothiocyanate (e.g., T16) e.g. for amines; amine (e.g., T17) e.g. for electrophiles including for peptide bond formation; NHS ester (e.g., T18) e.g. for peptide bond formation; benzylic halide, preferentially chloride, e.g. for thiol alkylation (e.g., T19); Michael acceptors e.g. maleimide (e.g., T20) or acrylates, e.g. for thiolate addition; thiol (e.g., T21) e.g. for conjugation to maleimide or ethynylphosphonamidates; pentafluorophenyl ring e.g. pentafluorobenzamide (e.g., T22) e.g. for reaction with thiolates and/or for labelling the cellular endoplasmic reticulum; halocarbonyl electrophiles e.g. iodoacetamides (e.g., T23) e.g. for reaction with thiols; as well as others that are well-known to those skilled in the art.

Further examples of the tether unit include a benzylguanine derivative for SNAP-tag labelling, a 6-chlorohexyl derivative for HaloTag-labelling, a benzylcytosine derivative for CLIP-tag labelling; or a strained alkene, alkyne, strained alkyne, azide, or tetrazine for click reactions; or an isothiocyanate, amine, NHS ester, benzylic halide, maleimide, acrylate, thiol, ethynylphosphonamidate, tetrafluorophenyl, pentafluorophenyl, 2-chloroacetamide, or 2-chloroacetate. In another aspect, the tether unit is biotin, desthiobiotin, a lipid motif containing 8 to 30 carbon atoms, a mitochondrial-targeting delocalised lipophilic cation including one based on a substituted triphenylphosphonium substituent, a DNA-binding motif, a taxane, a phalloidin, a jasplakinolide, or a short nucleic acid strand of 6 to 30 bases including a DNA strand. In a further aspect, the tether unit is a substrate for a self-labelling protein tag (such as a HaloTag, CLIP-tag, or SNAP-tag) or is a bioorthogonally or biologically reactive moiety that is also useful for covalently labelling biomolecules; or is a moiety that is useful for noncovalently targeting specific cellular regions or environments. In another embodiment, the tether unit is a benzylguanine derivative for SNAP-tag labelling, a 6-chlorohexyl derivative for HaloTag-labelling, or a benzylcytosine derivative for CLIP-tag labelling; and the fluorophore unit is from the xanthene-type series, the cyanine series or the polymethine series.

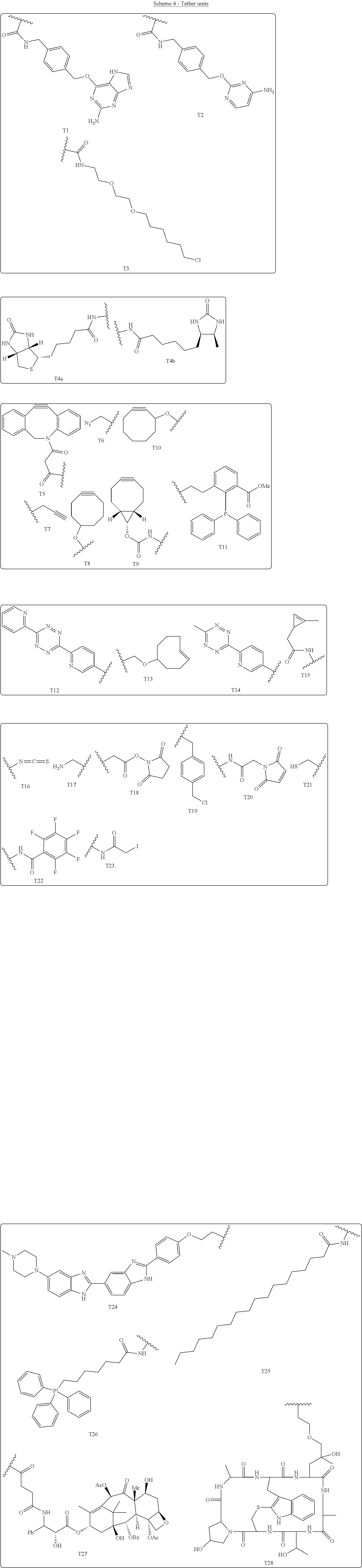

The aim for a noncovalent associating group is typically to achieve specific localisation to a target environment or a defined biological target. Therefore, such tether units can advantageously be chosen by those skilled in the art, including but not limited to: targeting lipid membranes or lipid droplets, e.g. by using a lipid anchor, such as in T25 or a cholesterol motif; or targeting mitochondria, e.g. by using a delocalised lipophilic cation, such as in T26; or targeting DNA, e.g. by using a DNA-associating molecular structure, such as an oligo-benzimidazole e.g. in T24; or the target may be a specific protein, which usually requires using a ligand of good protein-binding affinity (typically Kd below 1 μM), of which well-known examples are targeting the protein tubulin (e.g. by using taxane ligands e.g. in T27) so as to be able to image the microtubules that tubulin forms in cells; or targeting the protein actin (e.g. by using phalloidin or jasplankinolide ligands e.g. in T28) so as to be able to image the actin filaments that actin protein forms in cells; or targeting proteins such as streptavidin or neutravidin (e.g. by using biotin or desthiobiotin ligands e.g. in T4a and T4b) since these proteins are often either attached to structures such as DNA origami or beads, or are attached to other proteins (giving fusion proteins), and it can be advantageous to e.g. image those conjugates; and many other tethers that are widely-reported and well-known to those skilled in the art are also possible. A special tether unit type is a nucleic acid sequence such as a DNA or PNA sequence which can e.g. noncovalently associate to a complementary strand, in a manner that is either rapidly-reversible (e.g. 8-nucleotide sequences, see Examples) or else quasi-irreversible on the experimental timescale (e.g. 21-nucleotide sequences, see Examples). Compounds of the invention can be attached to nucleic acid sequences via intermediary tethering groups (see Examples), or can be directly built into the sequence by e.g. synthesising the compound's phosphoramidite derivative.

As shown in Scheme 4, the same variety of connection points introduced above can be used to connect a tether unit to other units within the compounds of the invention; see Examples.

Linker Unit: Linkers are motifs whose function is to stably and covalently connect two other units within a compound of the invention (e.g. linking fluorophore unit to azoaryl unit, or azoaryl unit to tether unit, etc). As such, they are not particularly limited in nature. Linkers can be flexible and/or freely rotatable, or can be rigidised; typically the linker is a bond or contains between 1 to 48 atoms along the shortest linear path between the two units that it connects, preferentially 1 to 24 atoms, more preferentially 1 to 20 atoms, even more preferentially 1 to 10 atoms. The atoms which are present in the shortest path can be selected from C, O, N and S. The linker unit can be a hydrocarbon linker unit which can optionally contain 1 to 16, preferably 1 to 8, more preferably 1 to 4, heteroatoms selected from N, O and S. Examples of linker units include, e.g. —(CH₂)ₜ— (with t being 1 to 20), —O(CH₂)ₜO— (with t being 1 to 6), —(O)C(CH₂)ₜC(O)— (with t being 1 to 6), —(O)C(CH₂)ₜC(O)— (with t being 1 to 6), —(CH₂CH₂O)ₙ— (with n being 1 to 6), poly(glycine) and —(C(O)CH₂NH)ₚ— (with p being 1 to 6), etc; examples are shown in Scheme 5. As is known to those skilled in the art, linkers include but are not limited to chains with linear or primarily linear connective topology, e.g. poly(ethyleneoxide) (also known as polyethylene glycol or PEG; —(CH₂CH₂O)ₙ—), or saturated hydrocarbon —(CH₂)ₙ—, or poly(glycine) —(C(O)CH₂NH)ₙ—. Linkers may be functionalised at their termini to allow forming connections to the units that they contact by more straightforward chemistries, such as the terminal amine functionalisation of the PEG linker in L1 which allows connection by e.g. urea or amide formation to azoaryl and/or tether units with suitable reactive functional groups as connection points (e.g. carboxylic acid or ester groups or an activated derivative of the same such as an N-hydroxysuccinimide (NHS) ester, or an aliphatic or aromatic primary or secondary amine); other useful connection points include a primary alkyne, an azide, or a maleimide. Linkers may also feature branch points, including but not limited to tertiary amines or tertiary amides or trisubstituted phenyl rings, which allow the one linker to connect three units to each other (e.g. L2 which connects unit R″, unit R′, and a HaloTag-reactive chlorohexyl tether unit), which can enable the formation of branched topologies between units within compounds of the invention, or can be employed to give dendrimeric structures (as with tertiary amine branch points connecting azoaryl species to give dendrimeric azoaryl unit D1 in Scheme 3).

It should be clear that the linker requirement for stable connection of fluorophore and azoaryl is an additional structural and functional differentiation from previously reported FRET probes, which typically require linker units that are to be cleaved in situ so that their fluorophore motif becomes separated from (unaffected by) their azoaryl motif (i.e. fluorescence is unquenched).

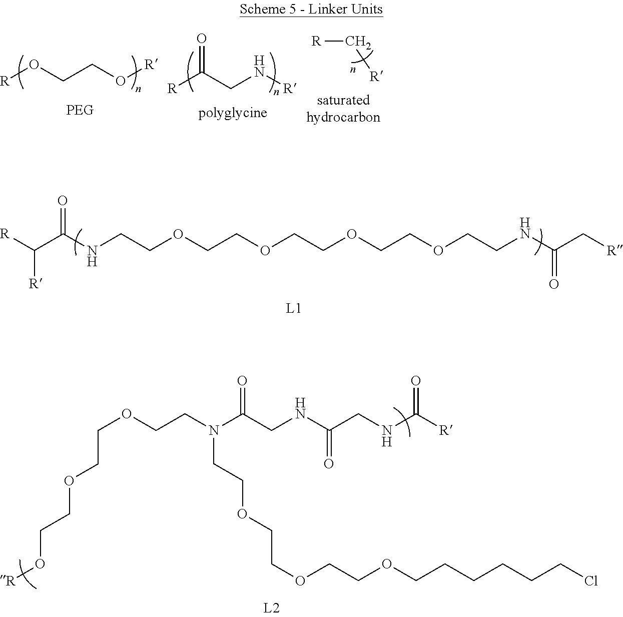

Compounds of the Invention: Compounds of the invention contain a fluorophore unit, an azoaryl unit, and optionally but advantageously a tether unit, connected by linker units. The chemistry of the individual units and of the connection points between these units has been discussed above. In Scheme 6 the nine topologies with which these units can be connected to each other are shown (P1-P8 for compounds containing a tether unit, P9 for compounds without a tether unit); in this scheme, “Azo” is an azoaryl unit; “Attach” is a tether unit; “F” is a fluorophore unit; linker units are drawn as straight or wavy lines; an intersection between straight and wavy lines marks a trifunctional branch point in a linker unit; an asterisk on a unit indicates that the unit is bivalent instead of monovalent in its connectivity. Specific examples of monovalent vs bivalent azoaryl units have been given; similar bivalent connectivities for fluorophore units and/or tether units are known to those skilled in the art. Preferably, the compound of the invention is P2, P3, P4, or P7, more preferably P2 or P4, since it is understood that these formulae permit the closest approach of the fluorophore to both the attachment site (important for super resolution imaging) and to the azoaryl (important for self-healing efficiency), while leaving the tether unit monovalent (can be chemically easier, cheaper, or more practical to work with).

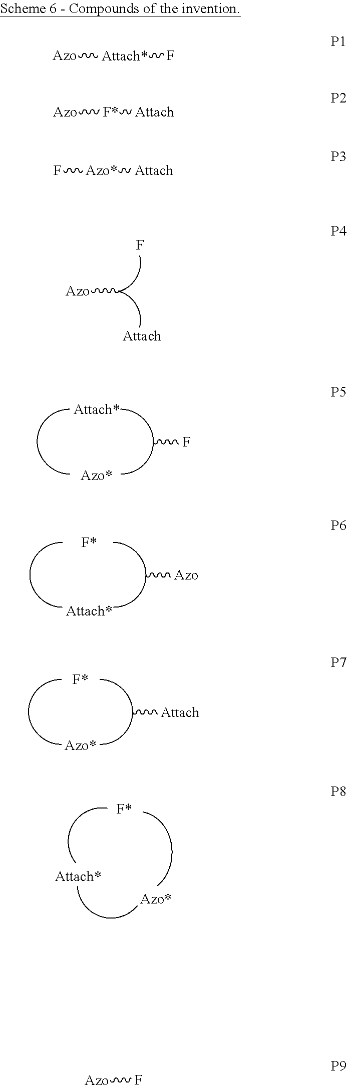

Scheme 7 illustrates compounds according to the invention. F1 uses a strained alkyne DBCO tether unit to covalently label e.g. an azide-labelled target, a bivalent bis(alkyl)-type azobenzene azoaryl unit, and a water-solubilised polysulfonated red/NIR Cy5 fluorophore unit. F2 uses a taxane tether unit to noncovalently bind to microtubules, a hetero-azoaryl, and a permanently fluorescent red/NIR silarhodamine fluorophore unit. F3 uses a maleimide covalent tether unit to label e.g. thiol targets, with a bis(alkoxy)azobenzene azoaryl unit and a solubilised AbberiorStar635P red fluorophore. F4 uses an NHS ester tether unit to covalently label e.g. amines, a bis-azobenzene azoaryl unit, and a Cy3B yellow fluorophore. F1-F4 all have a P3 topology with bivalent azoaryls. F5 uses a HaloTag-reactive chloroalkane tether unit to covalently label HaloTag protein domains or protein fusions, a bivalent carborhodamine green fluorophore, and a monovalent, dimeric azoaryl unit including a branch point tertiary amine, and has a P2 topology. F6 uses an Atto647 fluorophore unit, a bivalent tether unit based around a tetrazine for bioorthogonal ligation to e.g. targets bearing a trans-cyclooctene, and a tetra-ortho-substituted azoaryl unit, and has P1 topology. F7 uses a bivalent Cy7 NIR fluorophore unit, macrocyclically connected with a bivalent azoaryl unit, and a Staudinger ligation tether unit intended to react covalently with an azide, in P7 topology. F8 features a modified BODIPY fluorophore unit, with a branched linker unit that connects it to both the azoaryl and the tether unit, which is a biotin to bind noncovalently to avidin-type protein domains or fusions, in P4 topology.

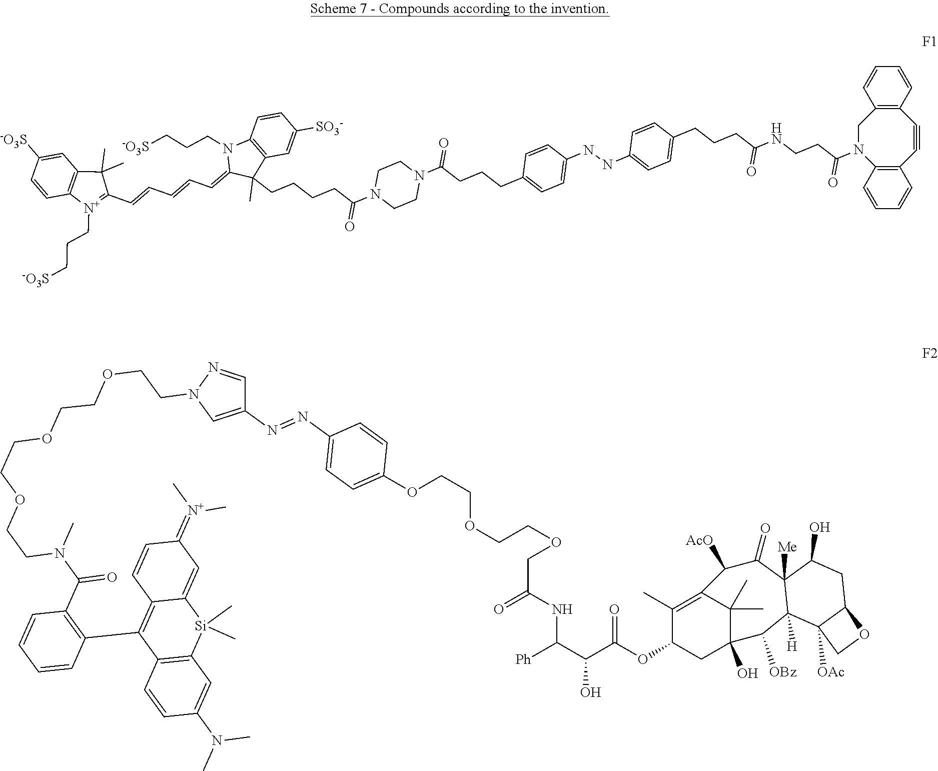

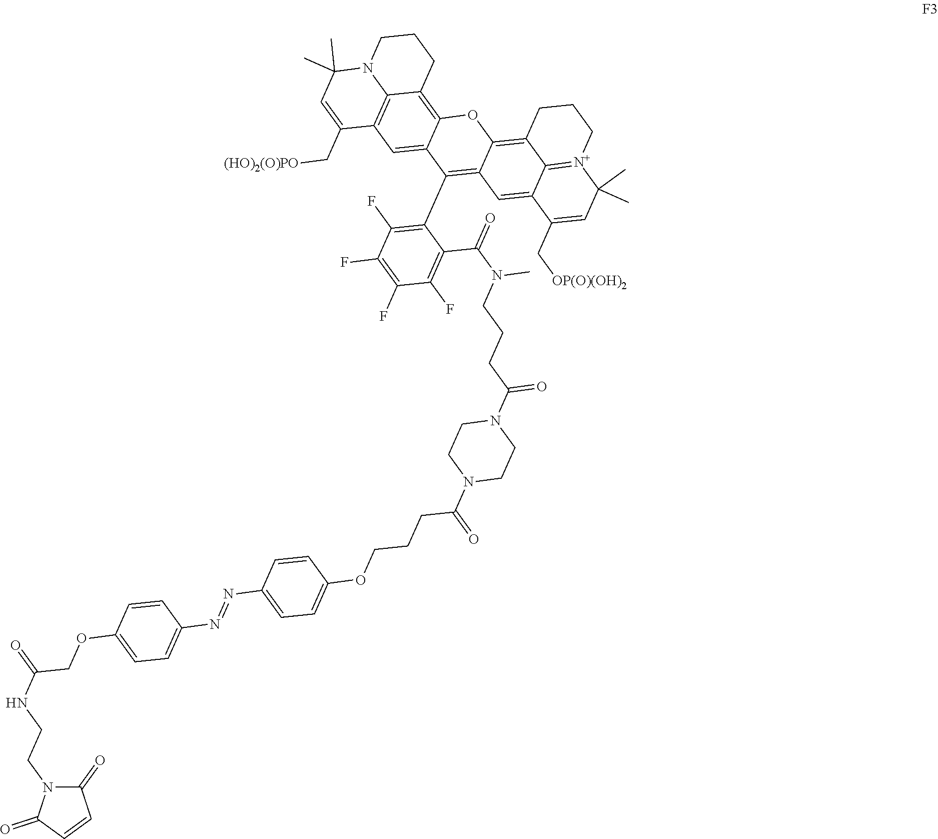

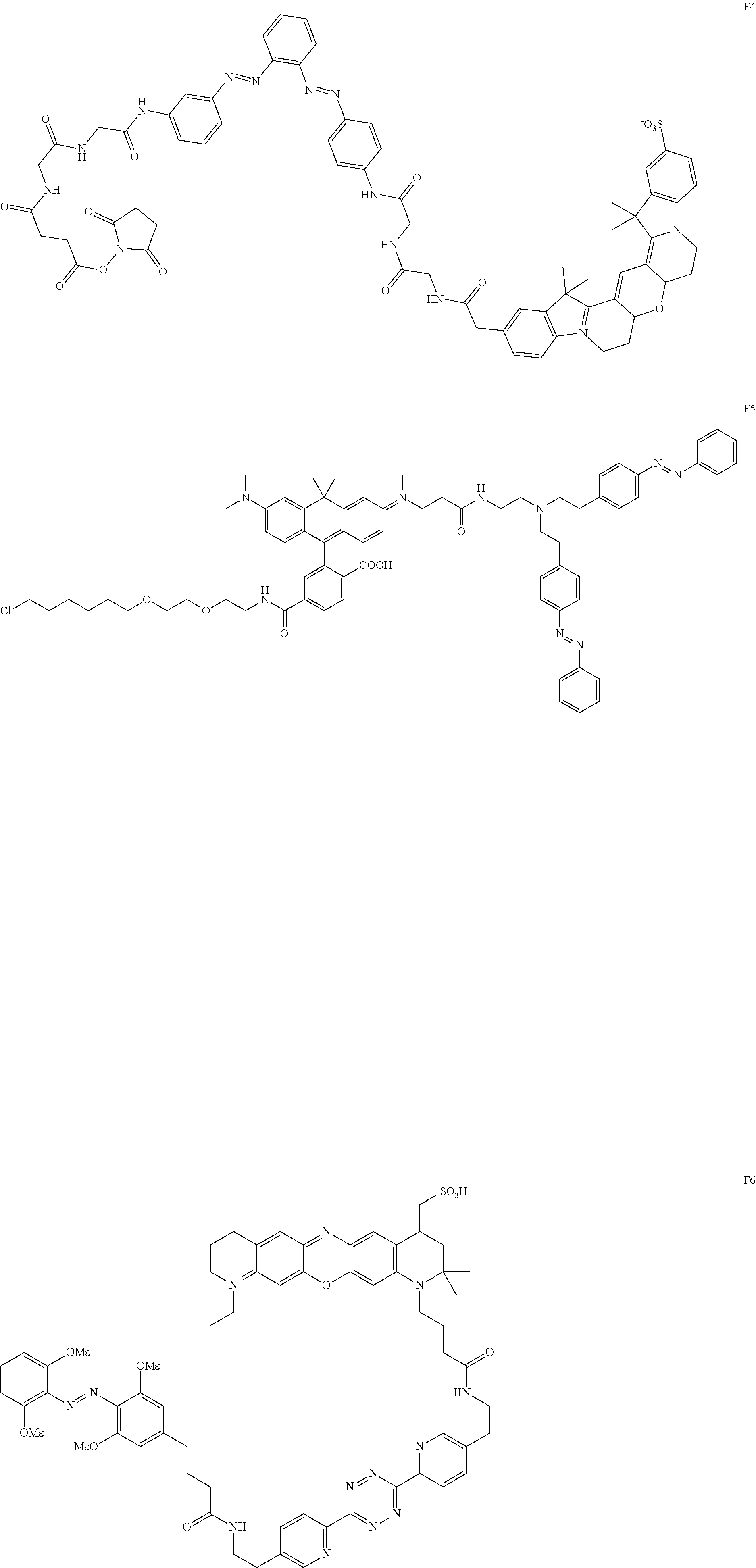

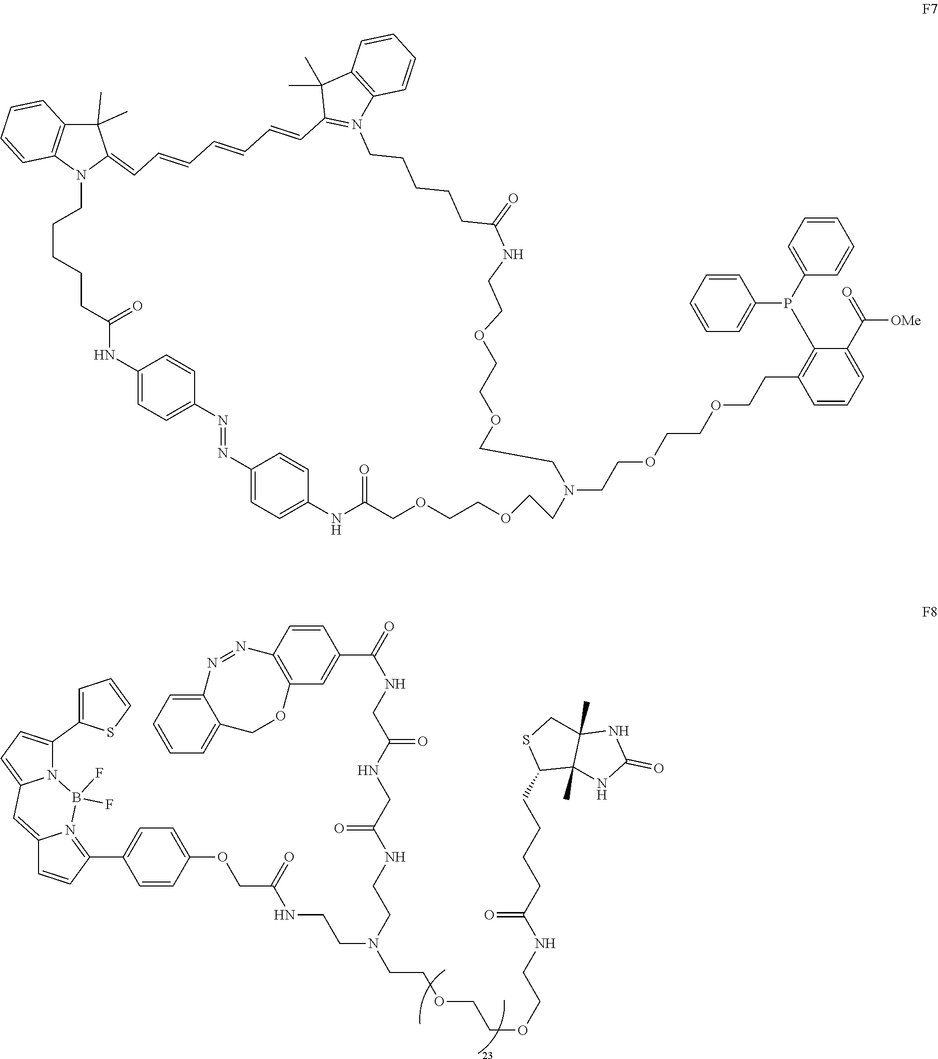

Scheme 8 illustrates selected compounds according to the invention for which results will be presented in the Examples, wherein Scheme 8a shows azoaryl units and cyanine-type fluorophore units used in the construction of molecules of the invention, and Scheme 8b shows a set of cyanine-based compounds according to the invention, each bearing a bioorthogonally ligatable tetrazine as a tether unit.

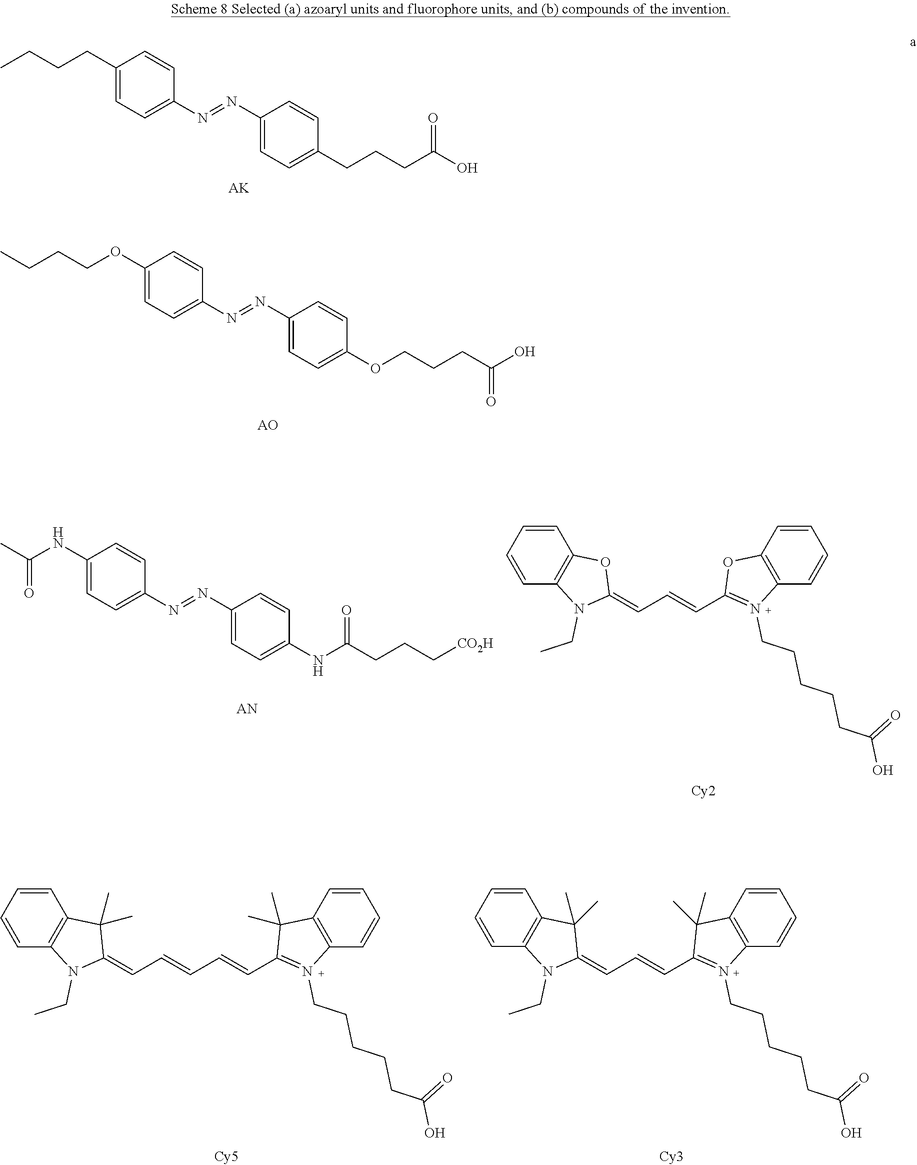

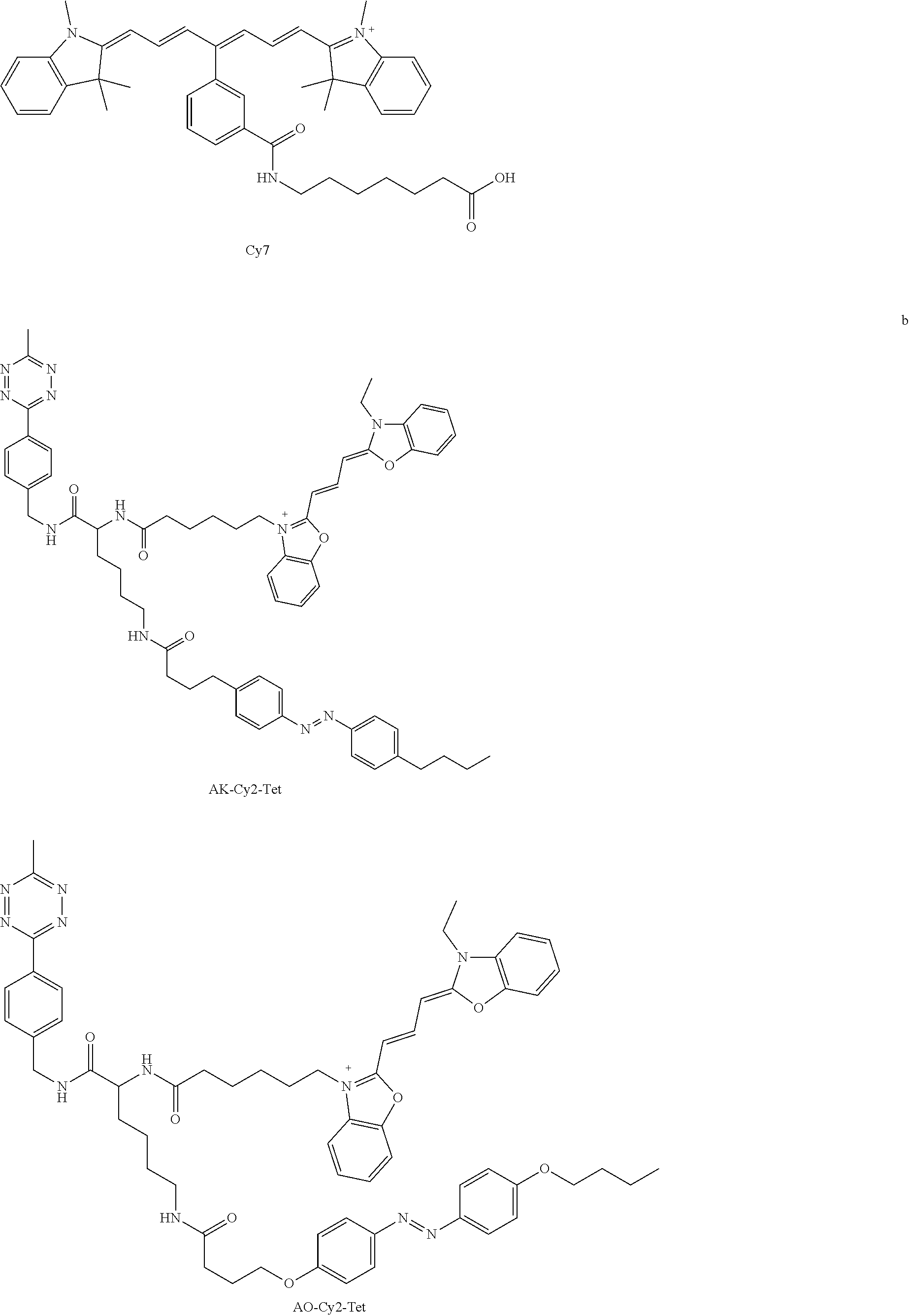

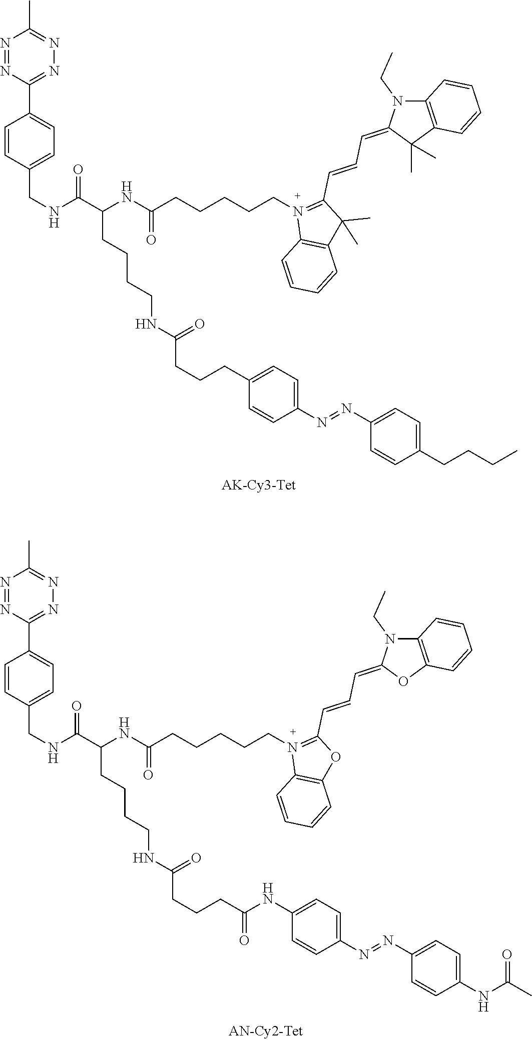

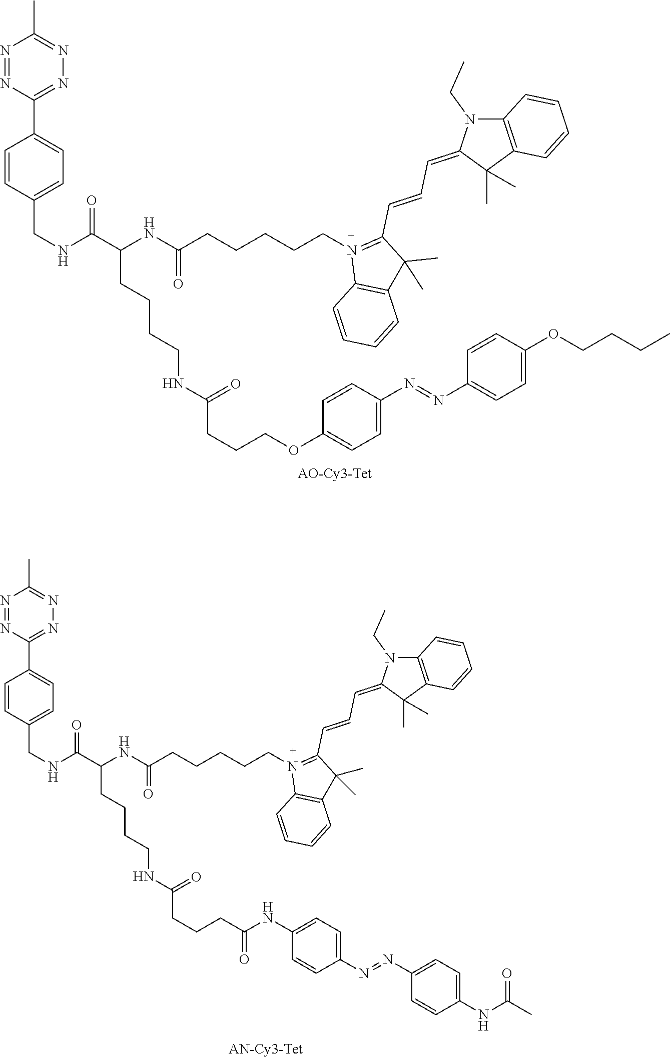

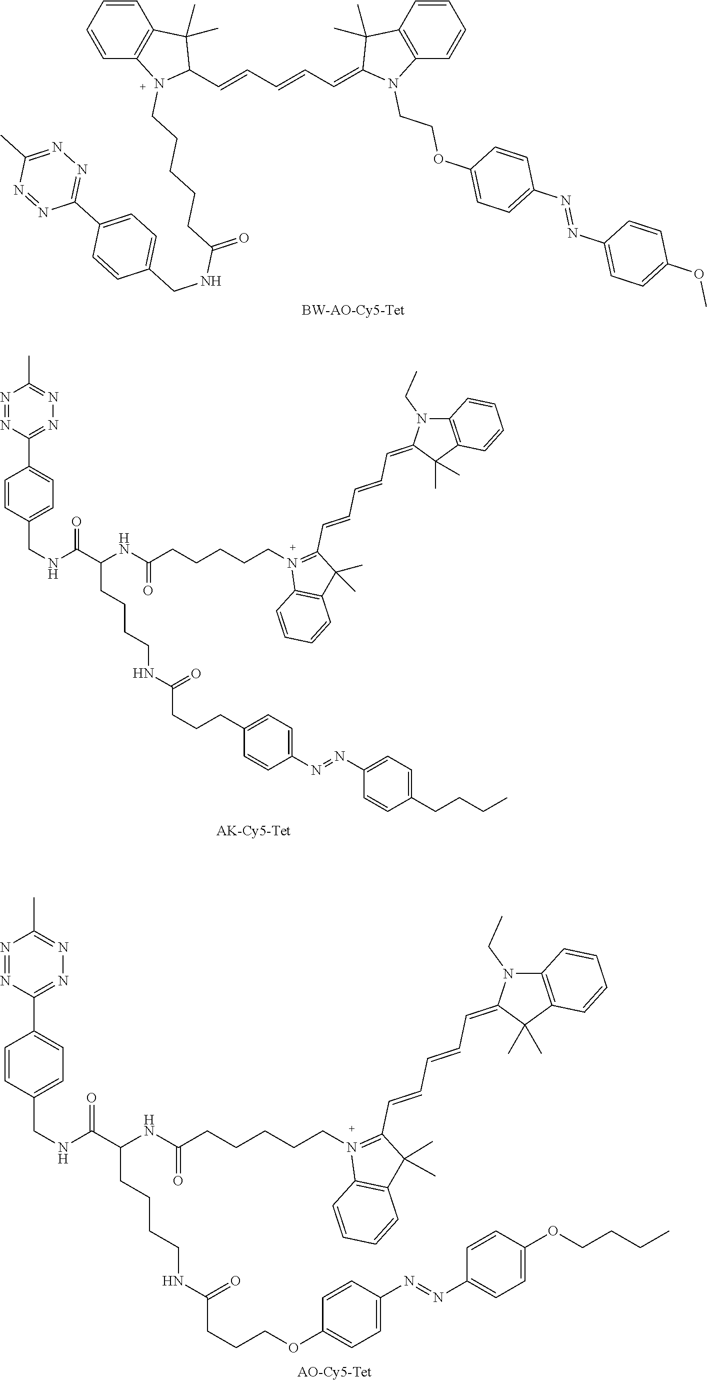

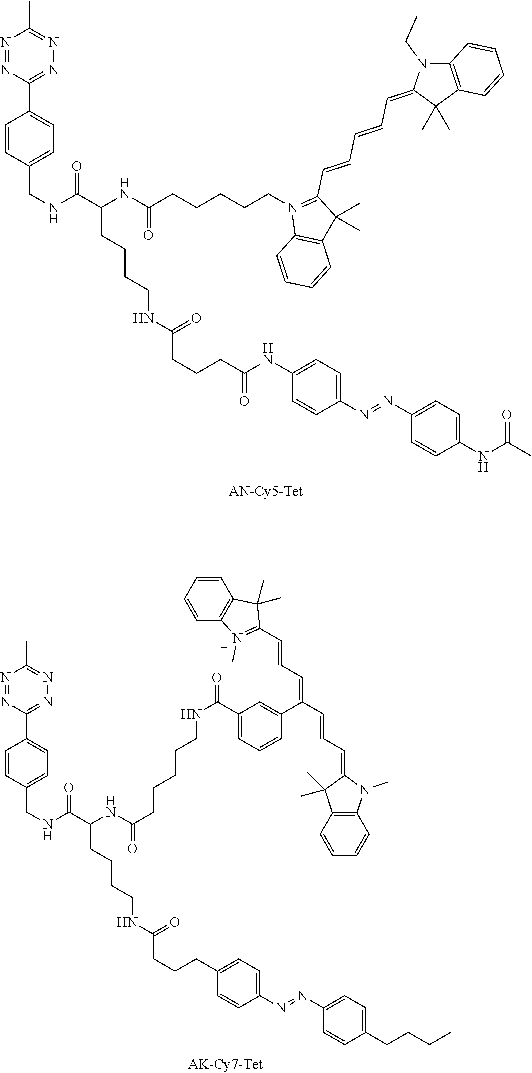

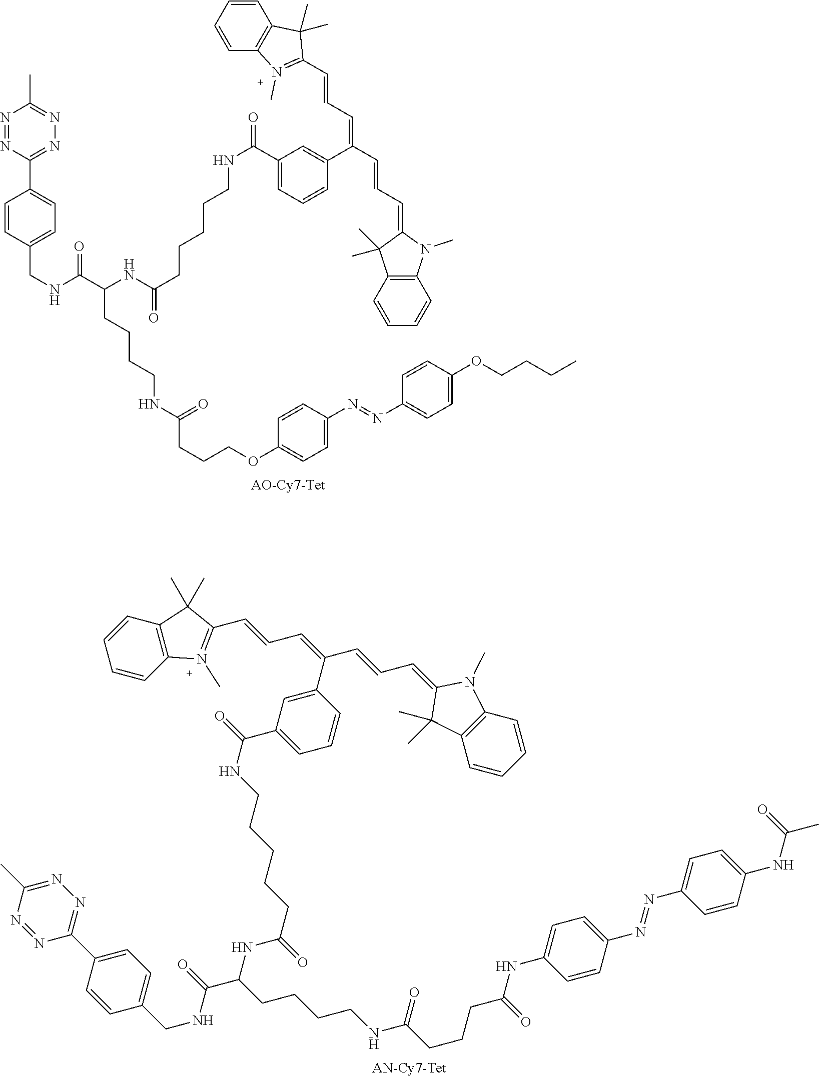

### Compound Having the Formula (I)

The present invention provides a compound of formula (I) that contains a fluorophore unit, an azoaryl unit, and optionally a tether unit. The formula (I) can be more precisely realised in the topological variants (1.1-1.8) and constitutional variant (1.9), wherein “Azo” is an azoaryl unit; “Attach” is a tether unit; “F” is a fluorophore unit; linker units are drawn as straight line or wavy line; an intersection between a straight and a wavy line marks a branch point in a linker; an asterisk on a unit indicates that the unit is bivalent instead of monovalent in its connectivity:

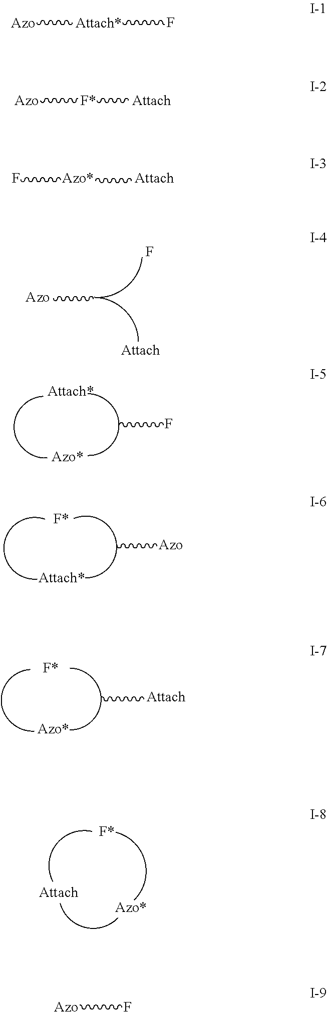

Classes and examples of fluorophore moieties suitable for use in the fluorophore unit have been described explicitly above; but any fluorophore compound derived by substitution of the same is also considered suitable for use. How acceptable compounds can be derived by substitution is known to those skilled in the art and will be understood by reference to the following non-limiting examples.

The compound's performance in fluorescence imaging is preferably improved in one or more of the following aspects as compared to the performance of the respective fluorophore F (hereafter denoted a “reference” fluorophore): higher resistance of the fluorophore motif to photobleaching, lower degree of photodamage caused by the fluorophore (e.g. by reducing the photogeneration of singlet oxygen in aerated imaging experiments), higher average instantaneous brightness, greater stability of the signal intensity, higher total photon budget, greater detection of localisations (i.e. more chance to detect a threshold number of fluorescence photons within a certain spatiotemporal window), greater spatial resolution especially in super resolution imaging by virtue of detecting a greater number of photons per emitter and per unit time, greater temporal resolution or framerate of imaging by virtue of more rapidly detecting photons.

When the fluorophore is from the xanthene class, this includes but is not limited to structures derived by substitutions of the xanthene scaffold i.e. general structures XL-1 (closed, nonfluorescent) and XL-2 (open, fluorescent) that can be in equilibrium with each other; illustratively, these generalised structures encompass derivatives such as XL-3 and XL-4:

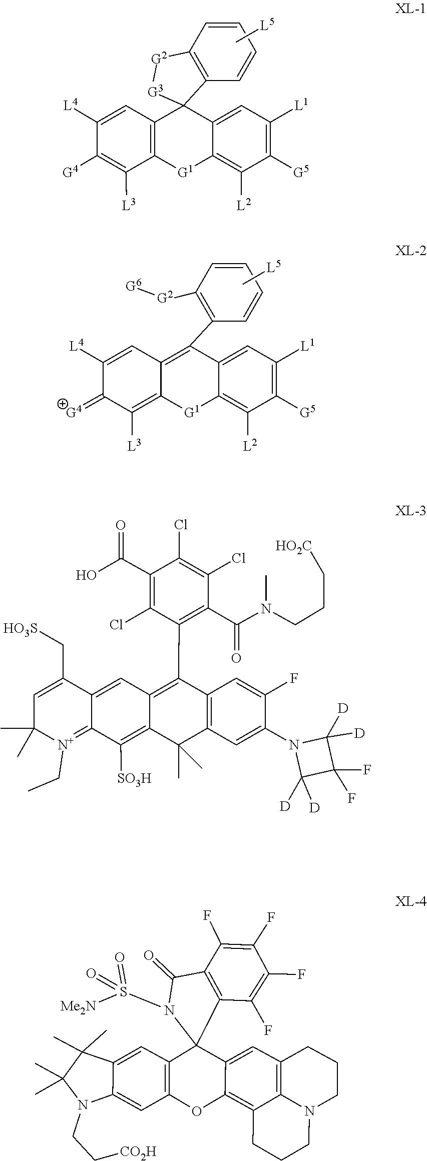

where
all “alkyl” or “alkylene” groups in a derivative [e.g. both alkyl groups in a “—C(—C₁₋₄-alkyl)₂-”] can be selected independently, and each can be optionally substituted by 1-12 heteroatom-containing moieties, wherein the heteroatoms are selected from O, N, S, Se, Si, Hal, B or P, with preferable heteroatom-containing moieties being sulfonic acid or phosphonic acid groups (—SO₃H, —PO₃H₂);
any hydrogen bound to carbon (C—H) in a derivative may be substituted by a deuterium (C-D) (deuteration), preferably at alkyl or alkylene groups of substituents G⁴, G⁵, L¹, L², L³ and/or L⁴, more preferably at G⁴ and G⁵;
L¹, L², L³ or L4 are independently selected from —H, -Hal, —NO₂, —SO₃H, —C₁₋₄-alkyl, —CN, —OMe, —OCF₃, and a —C₁₋₈-alkylene-group that connects to their neighbouring substituents G⁴ and/or G⁵ to form fused rings;
zero, one, two, three, or four substituents L⁵ can be attached at any free position on the indicated benzene ring, and are independently selected from —C₁₋₄-alkyl, —O—C₁₋₄-alkyl, —CO₂H, -Hal, —C(O)—C₁₋₄-alkyl, —C(O)—O—C₁₋₄-alkyl, —C(O)—N(—C₁₋₄-alkyl)₂, —C(O)—NH(—C₁₋₄-alkyl)₁, —SO₃H, —S(O)₂—N(—C₁₋₄-alkyl)₂, and —S(O)₂—NH(—C₁₋₄-alkyl);
G¹ is selected from —O—, —S—, —Se—, —C(—C₁₋₄-alkyl)₂-, —Si(—C₁₋₄-alkyl)₂-, —C(O)—, —CF₂—, —N(—C₁₋₄-alkyl)-, and —N(-phenyl);
G² is selected from —C(O)—, —S(O)₂—, —P(O)₂—, —CH₂—, and —CH(CH₃)—;
G³ is selected from —O—, —N(—C₁₋₄-alkyl)-, —NS(O)₂(—C₁₋₄-alkyl)-, —NS(O)₂(N(—C₁₋₄-alkyl)₂)—, —NS(O)₂(NH(—C₁₋₄-alkyl))-, —N(C≡N)—;
G⁴ and -G⁵ are independently selected from —O—H, —O—C₁₋₄-alkyl, —NH₂, —NH—C₁₋₄-alkyl, —N(—C₁₋₄-alkyl)₂, —N(—C₁₋₆-alkylene-) (e.g. —N-azetidinyl or —N-pyrrolidinyl), —N—C(O)—C₁₋₄-alkyl, noting that (i) the =G⁴ group in XL-2 may either be a tautomer of -G⁴ in XL-1 (e.g. ═O instead of —OH) or may bear an additional positive charge (e.g. =N+Me₂ instead of —NMe₂), and that (ii) when L¹, L², L³ or L4 are alkylene groups, each (G⁴ or G⁵)-alkylene bond replaces one (G⁴ or G⁵)-H or (G⁴ or G⁵)-C₁₋₄-alkyl bond in the above definition;
G⁶ in compounds that can undergo an open-closed equilibrium is either protonated -G³-H, or unprotonated -G³-: with a lone pair as indicated (e.g. —O—); but G⁶ in compounds that are exclusively in the open form can also be -G³-C₁₋₆-alkyl or —N(—C₁₋₈-alkylene-) (e.g. —N— azetidinyl or —N-pyrrolidinyl).

In one aspect, the derivative can contain two (e.g. XL-3) or one (e.g. XL-4) connection points, such as but not limited to —CO₂H, —NH₂, —C≡CH, and —N₃, independently chosen, that are required for assembling a compound of the invention; preferably, connection points are near to the π-system of the fluorescent form of the fluorophore, but are not in electronic conjugation to it.

It is understood that all combinations of the above definitions and preferred definitions are envisaged by the present inventors.

### Mechanism of Stabilisation

Without wishing to be bound by theory, it is assumed that when the fluorophore unit in a compound of the invention enters a (dark) triplet state, it then tends to undergo rapid triplet energy transfer to the azoaryl unit which returns the fluorophore unit to the ground state from where it can again engage in excitation/emissions cycles (bright state); while the triplet state azoaryl unit then undergoes rapid de-excitation to its ground state. It is assumed that the rate of triplet energy transfer to azoaryls is similarly good as or significantly better than to existing photostabilizers such as COT. It is assumed that, in particular, azoaryls have a uniquely rapid self-decay of the triplet state back to the singlet ground state, so they should act as a very fast and efficient triplet quenching agent which resists high-light-intensity operations well. Therefore, taken together, it is assumed that this mechanism can result in improvements of one or more imaging parameters, including photon output rate, signal stability, photon budget, survival time, instantaneous brightness, and localisation/detection rate, whether in oxygen-free conditions, or in aerated/oxygenated conditions including in living systems (which current photostabilisation concepts largely cannot address). Azoaryl electronics can be rationally tuned and it is further assumed that by exploring tuned structures, examples will be found that can be optimised to photostabilise even those fluorophores for which existing photostabilization strategies are usually not efficient (e.g. rhodamines).

### Methods

The present invention is also directed to fluorescence imaging methods in which the compounds of the invention are employed. Examples of fluorescence imaging methods in which the compounds of the present invention can be employed include fluorescence intensity imaging, fluorescence spectrum imaging, fluorescence lifetime imaging (FLIM), confocal or TIRF or superresolution (e.g. STED, MinSTED, PALM, dSTORM) imaging of fluorescence intensity; single-molecule FRET and/or multicolour FRET assays, single-particle tracking assays, calcium imaging assays, membrane potential imaging, or membrane fluidity or viscosity imaging.

Methods which Involve Labelling Ex Situ and then Imaging In Situ

In one aspect, the present invention is directed to a method of fluorescence imaging, wherein a compound of the present invention is labelled onto a target that is a biomolecule such as a protein, or an antibody, or a nanoparticle; and optionally the labelled object is then applied to a biological system such as cells in culture or a tissue slice, or else additional components are added to the labelled object such as additional proteins or small molecules for in vitro reconstitution assays; and the fluorescence emitted by the compound of the present invention under excitation illumination is detected.

Methods which Involve Labelling In Situ and then Imaging In Situ

In a further aspect, the present invention refers to a method of fluorescence imaging, wherein a compound of the present invention is introduced to a biological system such as cells in culture or a tissue slice or an animal so that the compound labels its target, such as a protein or an organelle, optionally by reaction with a self-labelling protein such as SNAP, Halo, or CLIP tag proteins, or optionally by bioorthogonal ligation chemistry such as a tetrazine-strained alkene-click reaction, and the fluorescence emitted by the compound of the present invention under excitation illumination is detected.

Methods which Involve Multiplexed Labelling and FRET Imaging

Another aspect of the present invention relates to a method of fluorescence imaging, wherein multiple compounds of claim 1, which are the same or different, are used to label multiple targets, and fluorescence emitted by one or more of the compounds of claim 1 is detected wherein optionally the fluorescence can be generated by direct photoexcitation of that compound, or else fluorescence is detected that is generated by fluorescence resonance energy transfer from a donor fluorophore that may or may not be a compound of the present invention to a compound of the present invention, or else fluorescence is detected that is generated by fluorescence resonance energy transfer from a compound of the present invention to another fluorophore that may or may not be a compound of the present invention.

This method can be used, for instance, to simultaneously image several different species in a sample.

### Methods of Fluorescence Imaging

A further aspect of the present invention refers to a method of fluorescence imaging, wherein a compound of the present invention is labelled onto a target by covalent reaction or by noncovalent association, and is either imaged directly or else the labelled target is brought into contact with additional assay components (such as being added to cells, or having proteins added to a solution) before imaging, wherein the imaging is done by photoexciting the compound of the present invention and detecting the fluorescence it emits by fluorescence intensity imaging, fluorescence spectrum imaging, fluorescence lifetime imaging (FLIM), confocal or TIRF or superresolution (e.g. STED, MinSTED, PALM, dSTORM) imaging of fluorescence intensity; single-molecule FRET and/or multicolour FRET assays, single-particle tracking assays, calcium imaging assays, membrane potential imaging, or membrane fluidity or viscosity imaging.

Another aspect of the present invention is directed to a method of fluorescence imaging, wherein a compound of the present invention is photoexcited and the fluorescence it emits is detected by imaging, wherein the imaging is preferably fluorescence intensity imaging, fluorescence spectrum imaging, fluorescence lifetime imaging (FLIM), confocal or TIRF or superresolution (e.g. STED, MinSTED, PALM, dSTORM) imaging of fluorescence intensity; single-molecule FRET and/or multicolour FRET assays, single-particle tracking assays, calcium imaging assays, membrane potential imaging, or membrane fluidity or viscosity imaging.

This method can, for instance, be used to monitor transport processes or to determine in which phase of a sample the compound of the present invention is present.

A further aspect of the present invention relates to a method of detecting a cellular process or protein or structure by fluorescence, the method comprising (i) administering to a cell, cell culture, or organism, a biomolecule such as a protein or an antibody that is labelled with a compound of the present invention, and (ii) detecting that biomolecule in the cell, cell culture, or organism, by the fluorescence of the compound of the invention.

Yet another aspect of the present invention is directed to a method for modifying the photophysical properties of a fluorophore, the method comprising covalently linking from one to four azoaryl units to the fluorophore, wherein the nature of the covalent linkage and/or the nature of the azoaryl unit is optionally varied in order to find a fluorophore-azoaryl compound structure with an optimal set of photophysical properties.

### Utility

The compounds of the invention are particularly useful as fluorescence imaging agents.

In one embodiment, the present invention provides a fluorescent dye for (typically in vitro) imaging of the structure, composition, or dynamics of a target species that may feature a permanent or exchangeable binding motif for the dye and/or for a carrier attached to the dye. Permanent binding motifs have been discussed in the section “tether units”. Exchangeable binding motifs can be used in procedures based around DNA-PAINT. The target species is not particularly limited; examples of targets include a protein, a nucleic acid, a biomolecule, a scaffold molecule, an antibody, an affibody, an aptamer, or an amino acid. Preferably, the target is a species for which it is valuable to track its spatial and/or temporal location. The image acquisition with the self-healing dye of the invention can be conducted as it would be for the parent dye; preferably, the imaging method involves high-excitation-intensity fluorescence imaging, as used in e.g. many single molecule and/or super-resolution imaging methods. The mode of imaging conducted can be e.g. fluorescence intensity imaging, fluorescence lifetime imaging, or fluorescence spectrum imaging, of the emitted intensity from the compound of the invention.

The compounds of the invention can be used in combination with one or more other imaging agents, diagnostics, theranostics, or therapeutics, whose nature is not particularly limited and will depend on the experiment being conducted. Preferably, one or more other imaging agents can be imaged independently from a compound of the invention, i.e. multiplexed multicolour imaging and/or lifetime imaging, to acquire multiple channels of information simultaneously. The imaging can also be performed where one or more of the other imaging agents are FRET donors or acceptors for the compound of the invention, particularly in the case that the other agent is covalently attached to the compound of the invention or if they are scaffolded in proximity to each other, or quenchers for the compound of the invention. Wherever multiple fluorescent agents are excited in an assay, each agent may be a compound of the invention.

The method of the above embodiments can be used as part of e.g. biochemical, biophysical and biological research in diverse settings, wherein such compounds of the invention are valuable optical probes. It is illustrated by Schemes 7-8 and the accompanying discussion, that a range of self-healing dyes with a range of structures, topologies, fluorescence properties, and physicochemical properties, including with extensive structural substitutions, are possible within the scope of the compounds of the invention. Various further modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention. The unity of all compounds of the invention is however ensured by the novel and coherent structure-based mechanism of functioning that the covalently-connected assembly of azoaryl unit, fluorophore unit, and optional tether unit delivers.

The improved fluorescence properties and reduced photodamage properties have great impact, particularly in assays where high-intensity excitation imaging is used, such as single-molecule imaging methods; examples of preferable methods settings include fluorescence lifetime imaging (FLIM) experiments, and confocal or TIRF or superresolution (e.g. STED, MinSTED, PALM, dSTORM) imaging of fluorescence intensity; examples of preferred application types include demanding imaging experiments such as single-molecule FRET and/or multicolour FRET assays; examples of preferred application settings include imaging objects that are highly sensitive to photosensitised damage (such as certain enzymes, motor proteins, and redox-active proteins) or that are located in environments that are highly sensitive to photosensitised damage (e.g. targets located at or within mitochondrial membranes or endoplasmic reticulum or plasma membrane) or where detection and imaging of only a few fluorophores must be performed (a situation in which maximised photon budget and maximised fluorophore photostability are crucial, and where high intensity excitation must often be used to collect signal on a practical timescale even though this can cause photodamage which it would be valuable to suppress); examples of preferred biological use applications where it is expected that azoaryl-photostabilised small molecule fluorophores of the invention will be valuable include imaging calcium concentration via photostabilised calcium sensors, imaging membrane voltage or other electrical properties, and imaging membrane order or tension. The compounds of the invention can be used in combination with one or more other imaging agents, diagnostics, theranostics, or therapeutics, whose nature is not particularly limited and will depend on the experiment being conducted. Preferably, one or more other imaging agents can be imaged independently from a compound of the invention, i.e. multiplexed multicolour imaging and/or lifetime imaging, to acquire multiple channels of information simultaneously. The imaging can also be performed where one or more of the other imaging agents are FRET donors or acceptors for the compound of the invention, particularly in the case that the other agent is covalently attached to the compound of the invention or if they are scaffolded in proximity to each other, or quenchers for the compound of the invention. Wherever multiple fluorescent agents are excited in an assay, each agent may be a compound of the invention.

### EXAMPLES

The invention will be more fully understood by reference to the following examples. They should not, however, be construed as limiting the scope of the invention.

### 1. Abbreviations

AcOH: acetic acid, Boc: tert-butyloxycarbonyl, calc.: calculated, DBCO: dibenzocyclooctyne, DBCO-amine: CAS number 1255942-06-3; 3-amino-1-[(5-aza-3,4:7,8-dibenzocyclooct-1-yne)-5-yl]-1-propanonedichloromethane), DCM: dichloromethane, DIPEA: diisopropylethylamine, DMF: dimethylformamide, DMSO: dimethylsulfoxide, EI: electron ionization, ESI: electron spray ionization, EtOAc: ethyl acetate, HBTU: 2-(1H-benzotriazol-1-yl)-1,1,3,3-tetramethyluronium hexafluorophosphate, iPrOH: propan-2-ol, J: coupling constant (in Hz), MeCN: acetonitrile, MeOH: methanol, NHS: N-hydroxysuccinimide, TEA: triethylamine, TFA: trifluoroacetic acid, THF: tetrahydrofuran, TSTU: N,N,N′,N′-tetramethyl-O—(N-succinimidyl)uronium tetrafluoroborate, THF: tetrahydrofuran, TLC: thin layer chromatography.

### 2. General Synthesis Conditions

Chemicals were obtained from Sigma-Aldrich, AttoTec, Abberior GmbH, TCI, Alfa Aesar, Acros, ABCR, or Carbolution, and were used as received without further purification unless stated otherwise. Unless stated otherwise, all reactions and characterisations were performed with solvents used as obtained, under closed air atmosphere without special precautions against air or moisture and were stirred with Teflon-coated magnetic stir bars. Reactions were monitored by thin layer chromatography (TLC) on Si60 F254 aluminium-backed sheets (Merck GmbH) and visualised by UV irradiation and/or KMnO₄ stain (3.0 g KMnO₄, 20 g K₂CO₃, 0.30 g KOH, 0.30 L H₂O). Flash column chromatography was conducted under positive nitrogen pressure using Ceduran® Si60 silica gel from Merck GmbH. TLC control, extractions and column chromatography were conducted using distilled, technical grade solvents as eluents. “Hexane” indicates a mixture of isomeric hexanes (e.g. 2-methylpentane, 3-methylpentane, 2,2-dimethylbutane, 2,3-dimethylbutane). Reaction yields refer to isolated chromatographically and spectroscopically pure materials, corrected for residual solvent content.

Analytical high performance liquid chromatography (HPLC) analysis was conducted using an Agilent 1100 system, through a Hypersil Gold HPLC column from ThermoFisher Scientific GmbH, with an inline DAD detector and unit resolution Agilent LC/MS IQ mass spectrometer (ESI mode). Mixtures of water (analytical grade, 0.1% formic acid) and MeCN (analytical grade, 0.1% formic acid) were used as eluent systems.

Nuclear magnetic resonance (NMR) spectroscopy was performed using Bruker Avance Ill HD Biospin spectrometers (¹H: 400 MHz/¹³C: 101 MHz, with BBFO cryoprobe™; or ¹H: 500 MHz/¹³C: 126 MHz). NMR spectra were measured at 298 K, unless stated otherwise, and were analysed with MestreNova 12. ¹H-NMR spectra chemical shifts (δ) in parts per million (ppm) relative to tetramethylsilane (δ=0 ppm) are reported using the residual protic solvent (CHCl₃ in CDCl₃: δ=7.26 ppm; CD₃SOCHD₂ in (CD₃)₂SO: δ=2.50 ppm; CHD₂OD in CD₃OD: δ=3.31 ppm; CHDCl₂ in CD₂Cl₂: δ=5.32 ppm; CHD₂CN in CD₃CN: δ=1.94 ppm; CD₃COCHD₂ in (CD₃)₂CO: δ=2.05 ppm) as an internal reference. For ¹³C-NMR spectra, chemical shifts in ppm relative to tetramethylsilane (δ=0 ppm) are reported using the central resonance of the solvent signal (CDCl₃: δ=77.2 ppm, (CD₃)₂SO: δ=39.5 ppm, CD₃OD: δ=49.0 ppm, CD₂Cl₂: δ=54.0 ppm, CD₃CN: δ=118.3 ppm, (CD₃)₂CO: δ=29.8 ppm) as an internal reference. For ¹H-NMR spectra, in addition to the chemical shift, the following peak data is reported in parenthesis: multiplicity, coupling constant(s) J (in Hz), and number of hydrogen atoms. The abbreviations for multiplicities and related descriptors are s=singlet, d=doublet, t=triplet, q=quartet, or combinations thereof, m=multiplet and br=broad. Where known products matched literature analysis data, reference for literature is given.

High-resolution mass spectrometry (HRMS) was conducted using a Thermo Finnigan LTQ FT Ultra FourierTransform ion cyclotron resonance spectrometer, applying electron spray ionisation (ESI) with a spray capillary voltage of 4 kV at temperature 250° C. with a method dependent range from 50 to 2000 u. All reported m/z values refer to positive ionization mode, unless stated otherwise.

### 3. General Synthesis Protocols (GPs)

### GP-A: Amide Couplings with Fluorophore NHS Ester and Azobenzene Amine

A fluorophore NHS ester (1 eq.) was dissolved in DMF (5 mg/mL) and charged with a solution of a DBCO-azoaryl-piperazine (1.1 eq., in DMF, 0.03 M) and DIPEA (3 eq.). The reaction mixture was vortexed and incubated at room temperature until full conversion of the starting material was confirmed by LC/MS. All volatile solvents were removed under a gentle stream of nitrogen and the crude material was used for coupling to DNA oligos without further purification.

### GP-B: Amide Couplings with Fluorophore Carboxylic Acid and Azobenzene Amine

A fluorophore carboxylic acid (1 eq.) was dissolved in DMF (5 mg/mL) and charged with TSTU (1.1 eq.) and DIPEA (5 eq., 10% in DMF). The reaction mixture was vortexed and incubated at room temperature for 1 h. A DBCO-azoaryl-piperazine (0.8 eq., in DMF 0.03 M) was added and the reaction mixture was vortexed and incubated at room temperature until full conversion of the starting material was confirmed by LC/MS. All volatile solvents were removed under a gentle stream of nitrogen. Typically, the crude product was later used for coupling to azide-labelled DNA oligos in a next step without further purification.

### GP-C: Preparation of Self-Healing Dyes with Branched Linker Unit

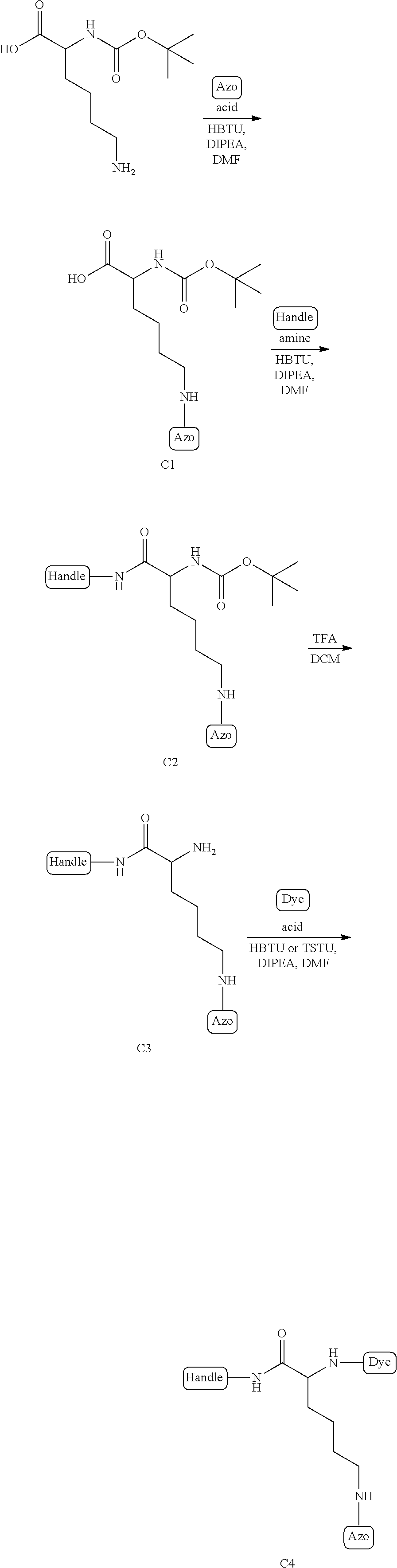

C1: An azobenzene carboxylic acid (1 eq.) was dissolved in DMF (0.10-0.25 M) and to the solution were added HBTU (0.95 eq.) and DIPEA (5.00 eq.). The reaction mixture was vortexed and incubated at room temperature for 10-30 min. Na-(tert-butoxycarbonyl)-L-lysine (1.10 eq.) in DMF (0.11-0.28 M) was added and the reaction mixture was vortexed and incubated at room temperature overnight. All volatile solvents were removed under a gentle stream of nitrogen and the crude material was purified by normal phase silica gel flash column chromatography with a DCM:MeOH eluent gradient to yield C1.

C2: An azobenzene-lysine carboxylic acid C1 (1 eq.) was dissolved in DMF (0.08-0.10 M) and to the solution were added HBTU (0.9-1.0 eq.) and DIPEA (5.0-7.0 eq.). The reaction mixture was vortexed and incubated at room temperature for 15-60 min. Commercial methyltetrazine-amine hydrochloride, CAS number 1596117-29-1 (0.9-1.0 eq.), neat or as a DMF solution (0.13 M), was added, and the reaction mixture was vortexed and incubated at room temperature overnight. All volatile solvents were removed under a gentle stream of nitrogen and the crude material was purified by normal phase silica gel flash column chromatography with a DCM:MeOH eluent gradient to yield C2.

C3: C2 (1 eq.) was dissolved in DCM to reach a concentration 0.05-0.07 M and trifluoroacetic acid (⅛ of the volume of DCM) was added. The reaction mixture was vortexed and incubated at room temperature until full conversion of the starting material was confirmed by LC/MS. All volatile solvents were removed under a gentle stream of nitrogen. For AO and AK compounds, the crude material C3 was used for subsequent experiments without further purification; whereas AN compounds were purified by normal phase silica gel flash column chromatography with a DCM:MeOH eluent gradient (to yield pure C3).

C4: To a fluorophore carboxylic acid (1.05-1.10 eq.) solution in DMF (ca. 0.03 M), was added a solution of either HBTU (1.0 eq., 13 mg/mL, for AN or AO compounds) or TSTU (1.0 eq., 17 mg/mL, for AK compounds) in DMF, followed by DIPEA (5.0 eq.). The reaction mixture was vortexed and incubated at room temperature for 15 min. A solution of C3 (0.90-0.95 eq.) and DIPEA (2.0 eq.) in DMF (100 μL) was added, the vial was washed with DMF (100 μL) and the reaction mixture was vortexed and incubated at room temperature for 1-5 h. The reaction mixture was purified by reverse phase preparative HPLC (water:MeCN gradients) to yield C4. Typically, the product C4 was used for coupling to trans-cyclooctene-labelled DNA oligos in the next step without further purification.

### 4. Synthesis

### 4.1 Synthesis of DBCO-AK-Piperazine

### 4-(4-((4-(4-methoxy-4-oxobutyl)phenyl)diazenyl)phenyl)butanoic acid (1)

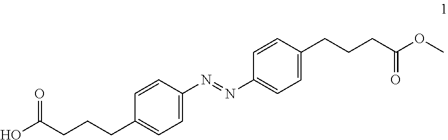 [SMILES: COC(=O)CCCc1ccc(/N=N/c2ccc(CCCC(=O)O)cc2)cc1]

A solution of Oxone® (5.73 g, 18.6 mmol, 9 eq.) in water (30 mL) was added to a solution of methyl 4-(4-aminophenyl)butanoate (Song; Journal of Enzyme Inhibition and Medicinal Chemistry 2020 35,1069-1079) (0.50 g, 2.6 mmol, 1.25 eq.) in DCM (30 mL, 0.09 M), and the biphasic reaction mixture was vigorously stirred at room temperature for 20 h. The phases were separated, the aqueous phase was extracted with DCM (3×60 mL) and the combined organic phases were washed with water (60 mL). 4-(4-aminophenyl)butanoic acid (0.37 g, 2.1 mmol, 1 eq.) and AcOH (100%, 30 mL) were added and stirred at room temperature for 15 min. The reaction mixture was then concentrated under reduced pressure, co-evaporated with toluene (2×20 mL) and purified by silica gel flash column chromatography (DCM/AcOH: 100/0 to 99/1) to yield 1 (0.64 g, 1.75 mmol, 84%) as an orange solid.

HRMS (ESI): m/z C₂₁H₂₃N₂O₄ [M−H]⁻: calc.: 367.16633, found: 367.16662.

¹H-NMR (400 MHz, methylene chloride-d₂) δ (ppm): 7.84 (dd, J=8.4, 1.2, 4H), 7.40-7.31 (m, 4H), 3.65 (s, 3H), 2.75 (dt, J=10.4, 7.6, 4H), 2.38 (dt, J=23.3, 7.4, 4H), 2.06-1.92 (m, 4H).

¹³C-NMR (101 MHz, methylene chloride-d₂)) δ (ppm): 179.1, 174.0, 151.6, 151.6, 145.5, 145.3, 129.6, 123.2, 123.2, 51.8, 35.3, 35.2, 33.7, 33.5, 26.8, 26.5.

### tert-butyl 4-(4-(4-((4-(4-methoxy-4-oxobutyl)phenyl)diazenyl)phenyl)butanoyl)piperazine-1-carboxylate (2)

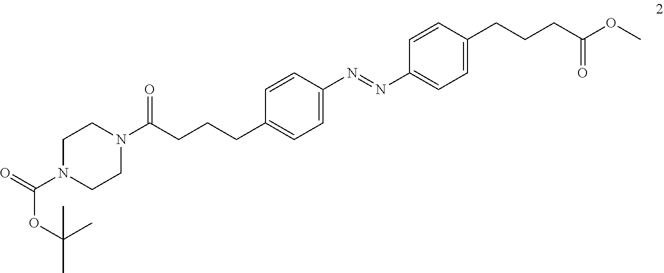 [SMILES: COC(=O)CCCc1ccc(/N=N/c2ccc(CCCC(=O)N3CCN(C(=O)OC(C)(C)C)CC3)cc2)cc1]

N-Boc-piperazine (303 mg, 1.63 mmol, 2.0 eq.), HBTU (371 mg, 0.97 mmol, 1.2 eq.) and TEA (0.79 mL, 5.7 mmol, 7 eq.) were added to a solution of 1 (300 mg, 0.81 mmol, 1 eq.) in DMF (25 mL, 0.03 M). The reaction mixture was stirred at room temperature for 16 h, concentrated under reduced pressure, and purified by silica gel flash column chromatography (hexanes/EtOAc: 90/10 to 70/30) to yield 2 as an orange oil (173 mg, 0.39 mmol, 49%).

HRMS (ESI): m/z C₂₅H₃₃N₄O₃⁺ [M-Boc+2H]⁺: calc.: 437.25472, found: 437.25462.

¹H-NMR (500 MHz, chloroform-d₁) δ (ppm): 7.85-7.81 (m, 4H), 7.32 (dd, J=8.3, 3.3, 4H), 3.68 (s, 3H), 3.62-3.34 (m, 8H), 2.75 (dt, J=17.0, 7.5, 4H), 2.39-2.28 (m, 4H), 2.08-1.96 (m, 4H), 1.46 (s, 9H).

¹³C-NMR (126 MHz, chloroform-d₁) δ (ppm): 174.0, 171.5, 154.7, 151.4, 151.4, 145.0, 144.9, 129.3, 129.3, 123.0, 80.5, 51.7, 45.5, 41.6, 35.3, 35.1, 33.5, 32.3, 28.5, 26.5, 26.4.

### 4-(4-((4-(4-(4-(tert-butoxycarbonyl)piperazin-1-yl)-4-oxobutyl)phenyl)diazenyl)phenyl)-butanoic acid (3)

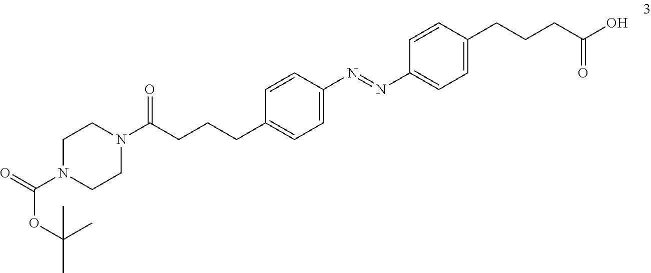 [SMILES: CC(C)(C)OC(=O)N1CCN(C(=O)CCCc2ccc(/N=N/c3ccc(CCCC(=O)O)cc3)cc2)CC1]

An aqueous solution of lithium hydroxide (15.0 mL, 2 M,) was added to a solution of 2 (150 mg, 0.28 mmol, 1 eq.) in MeOH (15 mL, 0.02 M). The reaction mixture was stirred at 50° C. for 2 h, adjusted to pH 6 using AcOH and extracted with EtOAc (3×150 mL). The combined organic phases were dried (Na₂SO₄), concentrated under reduced pressure and purified by silica gel flash column chromatography (EtOAc) to yield 3 (40 mg, 0.08 mmol, 27%) as an orange oil.

HRMS (ESI): m/z C₂₉H₃₇N₄O₅ [M−H]⁻: calc.: 521.27694, found: 521.27692.

¹H-NMR (400 MHz, acetone-d₆) δ (ppm): 7.90-7.81 (m, 4H), 7.48-7.40 (m, 4H), 3.55-3.47 (m, 4H), 3.45-3.34 (m, 4H), 2.81-2.74 (m, 4H), 2.45 (t, J=7.3, 2H), 2.37 (t, J=7.3, 2H), 2.02-1.91 (m, 4H), 1.44 (s, 9H).

¹³C-NMR (101 MHz, acetone-d₆) δ (ppm): 174.4, 171.5, 155.0, 152.0, 151.9, 146.8, 146.5, 130.2, 123.6, 123.6, 80.0, 45.9, 41.9, 35.8, 35.5, 33.5, 32.7, 28.5, 27.5, 27.3.

### tert-butyl 4-(4-(4-((4-(4-(3-(11,12-didehydrodibenzo[b,f]azocin-5(6H)-yl)-3-oxopropylamino)-4-oxobutyl)phenyl)diazenyl)phenyl)butanoyl)piperazine-1-carboxylate (4)

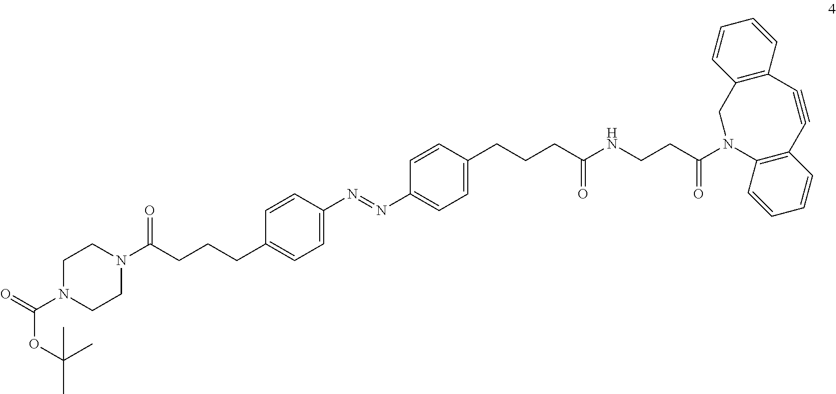 [SMILES: CC(C)(C)OC(=O)N1CCN(C(=O)CCCc2ccc(/N=N/c3ccc(CCCC(=O)NCCC(=O)N4Cc5ccccc5C#Cc5ccccc54)cc3)cc2)CC1]

DBCO-amine (15.0 mg, 0.05 mmol, 1 eq.), HBTU (30.9 mg, 0.08 mmol, 1.5 eq.) and TEA (45.5 μL, 0.30 mmol, 6 eq.) were added to a solution of 3 (34.0 mg, 0.07 mmol, 1.2 eq.) in DMF (2.5 mL, 0.03 M). The reaction mixture was stirred at room temperature for 16 h, concentrated under reduced pressure, and purified by silica gel flash column chromatography (DCM/MeOH: 100/0 to 97/3) to yield 4 (40 mg, 0.05 mmol, 95%) as a yellow oil.

HRMS (ESI): m/z C₄₇H₅₂N₆NaO₅⁺ [M+Na]⁺: calc.: 803.38969, found: 803.38985.

¹H-NMR (400 MHz, methylene chloride-d₂) δ (ppm): 7.87-7.77 (m, 4H), 7.69-7.57 (m, 2H), 7.46-7.21 (m, 9H), 7.19 (dd, J=7.3, 1.6 Hz, 1H), 6.18 (t, J=5.8 Hz, 1H), 5.10 (d, J=14.0 Hz, 1H), 3.67 (d, J=13.9 Hz, 1H), 3.53 (t, J=5.4 Hz, 2H), 3.41-3.32 (m, 6H), 3.34-3.07 (m, 2H), 2.73 (t, J=7.6 Hz, 2H), 2.60 (t, J=7.6 Hz, 2H), 2.34 (t, J=7.5 Hz, 2H), 2.06-1.88 (m, 6H), 1.86-1.74 (m, 2H), 1.43 (s, 9H).

¹³C-NMR (101 MHz, methylene chloride-d₂) δ (ppm): 173.2, 172.3, 171.8, 154.8, 151.5, 148.7, 145.6, 142.0, 132.5, 129.6, 129.5, 129.1, 128.8, 128.7, 128.4, 128.1, 127.5, 127.2, 125.9, 123.4, 123.2, 122.8, 118.3, 114.9, 110.8, 108.2, 80.3, 55.9, 54.4, 54.1, 53.8, 53.6, 53.3, 45.7, 41.8, 36.1, 35.8, 35.5, 35.4, 34.9, 32.7, 28.4, 27.3, 26.9.

### 4-(4-(4-((4-(4-(3-(11,12-didehydrodibenzo[b,f]azocin-5(6H)-yl)-3-oxopropylamino)-4-oxobutyl)phenyl)diazenyl)phenyl)butanoyl)piperazine (5, DBCO-AK-Piperazine)

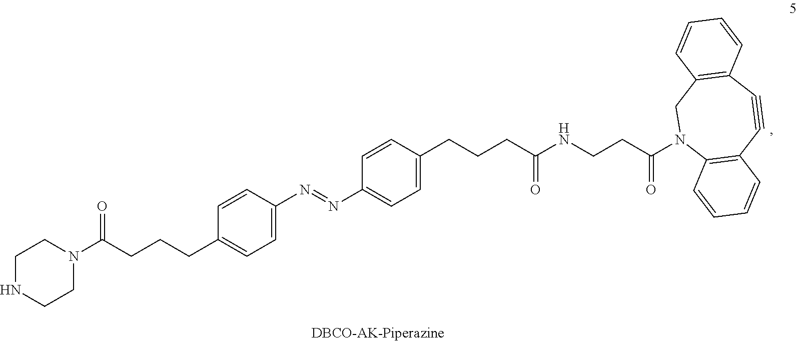 [SMILES: O=C(CCCc1ccc(/N=N/c2ccc(CCCC(=O)N3CCNCC3)cc2)cc1)NCCC(=O)N1Cc2ccccc2C#Cc2ccccc21]

TFA (400 μL) was added to a solution of 4 (18.5 mg, 0.02 mmol, 1 eq.) in DCM (600 μL, 0.04 M) and stirred at room temperature for 20 min. All volatiles were removed under a gentle stream of nitrogen to yield solid 5 (DBCO-AK-Piperazine) that was carried over directly for subsequent experiments without further purification.

HRMS (ESI): m/z C₄₂H₄₅N₆O₃⁺[M+H]⁺: calc.: 681.35476, found: 681.35480.

### 4.2 Synthesis of DBCO-AO-Piperazine

### tert-butyl 4-(2-(4-((4-hydroxyphenyl)diazenyl)phenoxy)acetyl)piperazine-1-carboxylate (6)

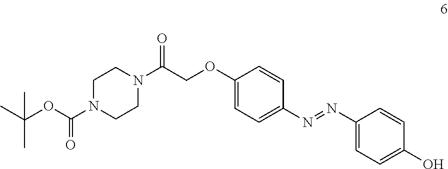 [SMILES: CC(C)(C)OC(=O)N1CCN(C(=O)COc2ccc(/N=N/c3ccc(O)cc3)cc2)CC1]

N-Boc-piperazine (273 mg, 1.47 mmol, 2 eq.), HBTU (334 mg, 0.88 mmol, 1.2 eq.) and TEA (0.79 mL, 5.7 mmol, 7 eq.) were added to a solution of 2-(4-((4-hydroxyphenyl)diazenyl)-phenoxy)acetic acid (Küllmer; Org. Biomol. Chem. 2022 20, 4204-4214) (200 mg, 0.74 mmol, 1 eq.) in DMF (15 mL, 0.05 M) and stirred at room temperature for 2 h. The mixture was concentrated under reduced pressure and purified by silica gel flash column chromatography (hexanes/EtOAc: 90/10 to 50/50) to yield 6 (269 mg, 0.61 mmol, 83%) as an orange/red oil.

HRMS (ESI): m/z C₂₃H₂₈N₄NaO₅⁺ [M+Na]⁺: calc.: 463.19574, found: 463.19516.

¹H-NMR (400 MHz, acetone-d₆) δ (ppm): 7.88-7.76 (m, 4H), 7.17-7.09 (m, 2H), 7.03-6.95 (m, 2H), 4.98 (s, 2H), 3.63-3.39 (m, 8H), 1.45 (s, 9H).

¹³C-NMR (101 MHz, acetone-d₆) δ (ppm): 166.8, 161.2, 160.9, 155.0, 148.2, 147.1, 125.4, 124.8, 116.5, 116.0, 80.1, 67.7, 45.7, 42.3, 28.5.

### tert-butyl 4-(2-(4-((4-(4-ethoxy-4-oxobutoxy)phenyl)diazenyl)phenoxy)acetyl)piperazine-1-carboxylate (7)

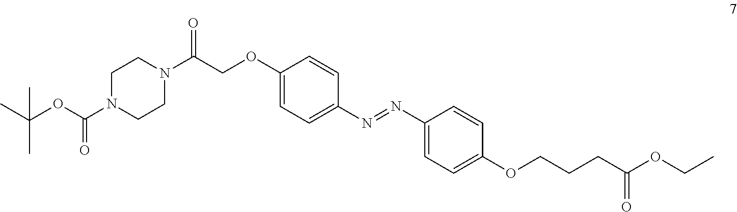 [SMILES: CCOC(=O)CCCOc1ccc(/N=N/c2ccc(OCC(=O)N3CCN(C(=O)OC(C)(C)C)CC3)cc2)cc1]

Ethyl 4-bromobutyrate (0.29 mL, 2.04 mmol, 4.5 eq.) was added to a mixture of 6 (200 mg, 0.45 mmol, 1 eq.) and K₂CO₃ (126 mg, 0.91 mmol, 2 eq.) in acetone (15 mL, 0.03 M). The mixture was stirred at 60° C. for 16 h, concentrated under reduced pressure and purified by silica gel flash column chromatography (DCM/MeOH: 100/0 to 99/1) to yield 7 (50 mg, 0.09 mmol, 20%) as an orange solid.

HRMS (ESI): m/z C₂₈H₃₈N₄O₇⁺ [M+H]⁺: calc.: 555.28133, found: 555.28130.

¹H-NMR (400 MHz, methylene chloride-d₂) δ (ppm): 7.91-7.82 (m, 4H), 7.08-6.98 (m, 4H), 4.78 (s, 2H), 4.20-4.06 (m, 4H), 3.60-3.40 (m, 8H), 2.52 (t, J=7.3, 2H), 2.18-2.07 (m, 2H), 1.45 (s, 9H), 1.32-1.19 (m, 3H).

¹³C-NMR (101 MHz, methylene chloride-d₂) δ (ppm): 173.3, 166.3, 161.6, 160.2, 154.7, 148.0, 147.3, 124.8, 124.7, 115.3, 115.1, 80.3, 68.0, 67.6, 60.8, 45.5, 42.2, 31.1, 28.4, 25.0, 14.4.

### 4-(4-((4-(2-(4-(tert-butoxycarbonyl)piperazin-1-yl)-2-oxoethoxy)phenyl)diazenyl)phenoxy)-butanoic acid (8)

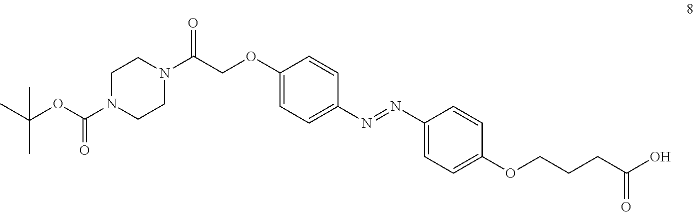 [SMILES: CC(C)(C)OC(=O)N1CCN(C(=O)COc2ccc(/N=N/c3ccc(OCCCC(=O)O)cc3)cc2)CC1]

An aqueous solution of lithium hydroxide (1.0 M, 0.51 mL, 0.51 mmol, 10 eq.) was added to a solution of 7 (28 mg, 0.05 mmol, 1 eq.) in THF (1 mL, 0.05 M). The reaction mixture was stirred at room temperature for 1 h, adjusted to pH 7 with an aqueous NH₄Cl solution (2 M), and extracted with a DCM:JPrOH mixture (3:1, 3×20 mL). The combined organic phases were dried (Na₂SO₄) and concentrated under reduced pressure to yield 8 (21 mg, 0.04 mmol, 77%) as a yellow/orange oil.

HRMS (ESI): m/z C₂₇H₃₅N₄O₇⁺ [M+H]⁺: calc.: 527.25003, found: 527.24991.

¹H-NMR (400 MHz, acetone-d₆) δ (ppm): 7.97-7.75 (m, 4H), 7.15-7.08 (m, 4H), 4.97 (s, 2H), 4.17 (t, J=6.3, 2H), 3.66-3.29 (m, 8H), 2.53 (t, J=7.3, 2H), 2.15-2.07 (m, 2H), 1.45 (s, 9H).

### tert-butyl 4-(2-(4-((4-(4-((3-(11,12-didehydrodibenzo[b,f]azocin-5(6H)-yl)-3-oxopropyl)amino)-4-oxobutox phenyl)diazenyl)phenoxy)acetyl)piperazine-1l-carboxylate (9)

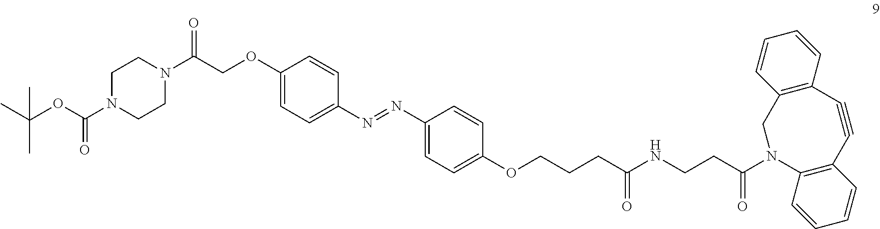 [SMILES: CC(C)(C)OC(=O)N1CCN(C(=O)COc2ccc(/N=N/c3ccc(OCCCC(=O)NCCC(=O)N4Cc5ccccc5C#Cc5ccccc54)cc3)cc2)CC1]

DBCO-amine (11.5 mg, 0.04 mmol, 1.05 eq.), HBTU (22.5 mg, 0.06 mmol, 1.5 eq.) and TEA (38.5 μL, 0.27 mmol, 7 eq.) were added to a solution of 8 (21 mg, 0.04 mmol, 1 eq.) in DMF (4 mL, 0.01 M), and stirred at room temperature for 1 h. The reaction mixture was concentrated under reduced pressure and purified via silica gel flash column chromatography (DCM/MeOH: 100/0 to 98/2) to yield 9 (19 mg, 0.02 mmol, 44%) as a yellow/orange oil.

HRMS (ESI): m/z C₄₅H₄₉N₆O₇⁺[M+H]⁺: calc.: 785.36572, found: 785.36518.

¹H-NMR (400 MHz, chloroform-d₁) δ (ppm): 7.91-7.84 (m, 4H), 7.81-7.73 (m, 2H), 7.66 (d, J=7.5 Hz, 1H), 7.43-7.33 (m, 5H), 7.04 (d, J=9.0 Hz, 2H), 6.98 (d, J=9.0 Hz, 2H), 6.18 (t, J=6.0 Hz, 1H), 5.11 (d, J=14.0 Hz, 1H), 4.78 (s, 2H), 4.00 (t, J=6.0 Hz, 2H), 3.69 (d, J=13.9 Hz, 1H), 3.63-3.53 (m, 4H), 3.50-3.35 (m, 8H), 2.23 (t, J=7.4 Hz, 2H), 2.02 (dt, J=14.5, 7.0 Hz, 2H), 1.46 (s, 9H).

¹³C-NMR (101 MHz, chloroform-d₁) δ (ppm): 172.5, 166.6, 161.2, 159.5, 154.6, 151.1, 148.1, 147.1, 140.1, 132.2, 129.1, 128.8, 128.7, 128.5, 128.0, 127.4, 126.7, 125.8, 124.6, 123.0, 122.6, 116.9, 115.0, 114.9, 111.6, 107.9, 80.7, 68.1, 67.4, 55.7, 45.5, 42.2, 35.5, 34.9, 32.9, 28.5, 25.2.

### N-(3-(11,12-didehydrodibenzo[b,f]azocin-5(6H)-yl)-3-oxopropyl)-4-(4-((4-(2-oxo-2-(piperazin-1-yl)ethoxy)phenyl)diazenyl)phenoxy)butanamide (10, DBCO-AO-Piperazine)

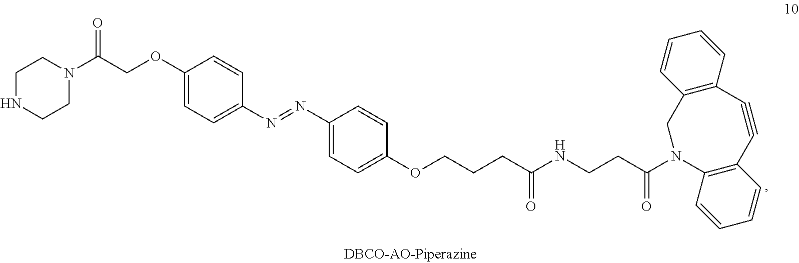 [SMILES: O=C(CCCOc1ccc(/N=N/c2ccc(OCC(=O)N3CCNCC3)cc2)cc1)NCCC(=O)N1Cc2ccccc2C#Cc2ccccc21]

TFA (100%, 80 μL, 1.27 mmol, 80 eq.) was added to a solution of 9 (10.0 mg, 12.7 μmol, 1 eq.) in DCM (120 μL, 0.11 M) and stirred at room temperature for 15 min. All volatiles were removed under a gentle stream of nitrogen. 10 (DBCO-AO-Piperazine) was obtained as an orange/red oil that was used for the next step without further purification.

HRMS (ESI): m/z C₄₀H₄₁N₆O₅ [M+H]⁺: calc.: 685.31329, found: 685.31282.

### 4.3 Synthesis of DBCO-Azoaryl-Piperazine-Dye Conjugates

### 2-((1 E,3E)-5-((Z)-1-(6-(4-(4-(4-((4-(4-((3-(11,12-didehydrodibenzo[b,f]azocin-5(6H)-yl)-3-oxopropyl)amino)-4-oxobutyl)phenyl)diazenyl)phenyl)butanoyl)piperazin-1-yl)-6-oxohexyl)-3,3-dimethylindolin-2-ylidene)penta-1,3-dien-1-yl)-1-ethyl-3,3-dimethyl-3H-indol-1-ium (11, Cy5-AK-DBCO)

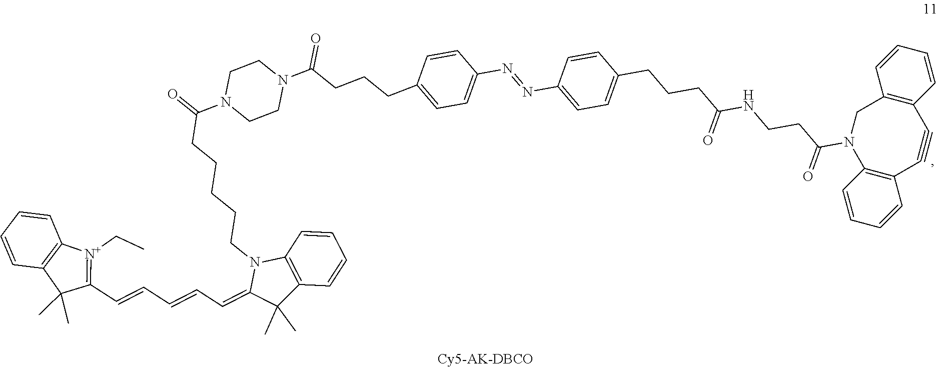 [SMILES: CC[N+]1=C(/C=C/C=C/C=C2\N(CCCCCC(=O)N3CCN(C(=O)CCCc4ccc(/N=N/c5ccc(CCCC(=O)NCCC(=O)N6Cc7ccccc7C#Cc7ccccc76)cc5)cc4)CC3)c3ccccc3C2(C)C)C(C)(C)c2ccccc21]

11 was synthesized according to GP-B, using:
as fluorophore acid: “Cy5 acid” (Pisoni; J. Org. Chem. 2014 79, 5511-5520), CAS number: 756457-35-9 (1.5 mg, 3.02 μmol, 1 eq.); and
as DBCO-azoaryl-piperazine: 5 (82 μL, 0.03 M in DMF, 2.42 μmol, 0.8 eq.).

HRMS (ESI): m/z C₇₅H₈₃N₈O₄⁺[M]⁺: calc.: 1159.65318, found: 1159.65425.

### N-(3-(11,12-didehydrodibenzo[b,f]azocin-5(6H)-yl)-3-oxopropyl)-4-(4-(4-(4-(4-Atto542-piperazin-1-yl)-4-oxobutyl)phenyl)diazenyl)phenyl)butanamide (12, Atto542-AK-DBCO)

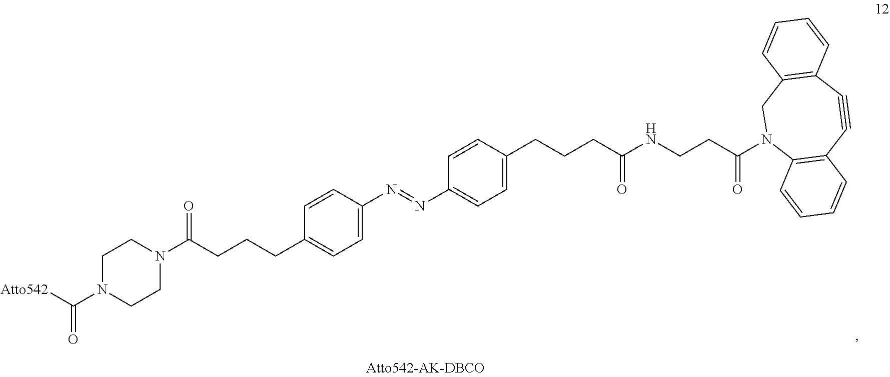

12 was synthesized according to GP-A, using:
as fluorophore NHS ester: commercial “Atto542 NHS ester” (1.0 mg, 0.89 μmol, 1 eq.)
as DBCO-azoaryl-piperazine: 5 (34 μL, 0.03 M in DMF, 0.98 μmol, 1.1 eq.)

HRMS (ESI): Atto542 molecular formula is not reported, but using the quoted mass after coupling, expected m/z [M]⁺ is 1576; found: 1576.4974.

### N-(3-(11,12-didehydrodibenzo[b,f]azocin-5(6H)-yl)-3-oxopropyl)-4-(4-((4-(4-(4-Cy3B-piperazin-1-yl)-4-oxobutyl)phenyl)diazenyl)phenyl)butanamide (13, Cy3B-AK-DBCO)

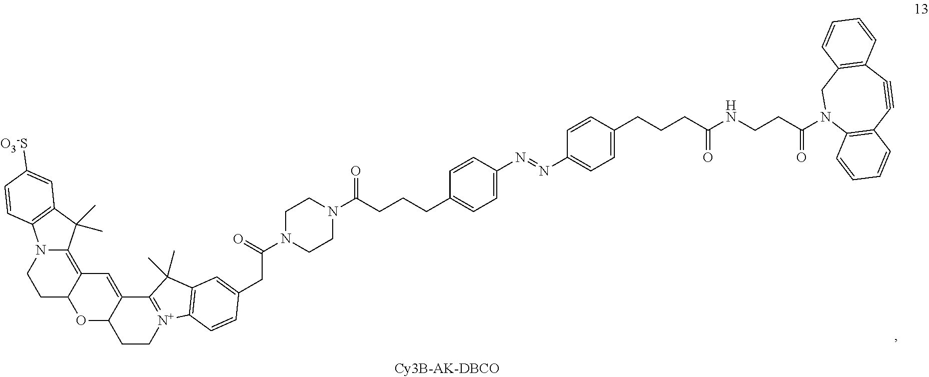

13 was synthesized according to GP-A, using:
as fluorophore NHS ester: commercial “Cy3B NHS ester” CAS number 228272-52-4 (1.0 mg, 1.52 μmol, 1 eq.),
as DBCO-azoaryl-piperazine: 5 (57 μL, 0.03 M in DMF, 1.67 μmol, 1.1 eq.).

HRMS (ESI): m/z C₇₃H₇₆N₈O₈S²⁺[M+2H]²⁺: calc.: 612.27479, found: 612.2762.

### 2-((1 E,3E)-5-((Z)-3-(5-(4-(4-(4-(4-(4-((3-(11,12-didehydrodibenzo[b,f]azocin-5(6H)-yl)-3-oxopropyl)amino)-4-oxobutyl)phenyl)diazenyl)phenyl)butanoyl)piperazin-1-yl)-5-oxopentyl)-3-methyl-5-sulfonato-1-(3-sulfonatopropyl)indolin-2-ylidene)penta-1,3-dien-1-yl)-3,3-dimethyl-1-(3-sulfonatopropyl)-3H-indol-1-ium-5-sulfonate (14, AF647-AK-DBCO)

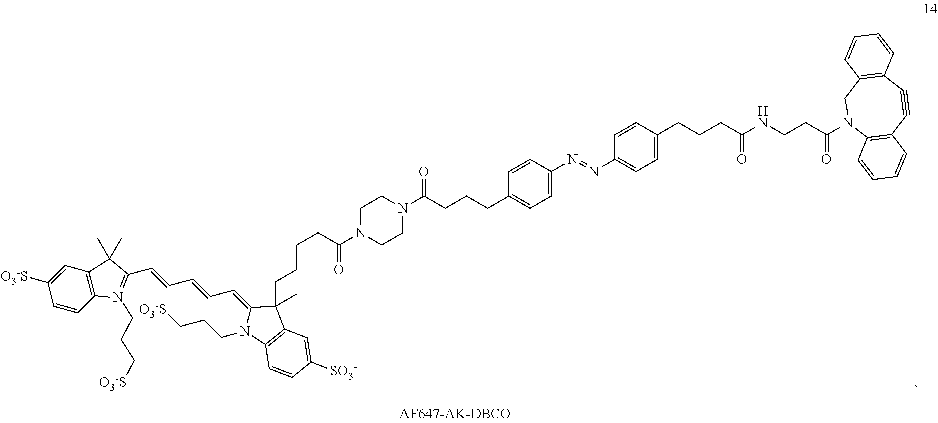

14 was synthesized according to GP-A, using:
as fluorophore NHS ester: commercial “AlexaFluor647 NHS ester”, CAS number 1620475-28-6 (1.0 mg, 0.95 μmol, 1 eq.)
as DBCO-azoaryl-piperazine: 5 (30 μL, 0.03 M in DMF, 0.88 μmol, 0.9 eq.)

HRMS (ESI): m/z C₇₇H₈₃N₈O₁₆S₄³⁻ [M]³⁻: calc.: 501.16088, found: 501.16091.

### N-(3-(11,12-didehydrodibenzo[b,f]azocin-5(6H)-yl)-3-oxopropyl)-4-(4-((4-(4-(4-AbberiorStar635P-piperazin-1-yl)-4-oxobutyl)phenyl)diazenyl)phenyl)butanamide (15, AbberiorStar635P-AK-DBCO)

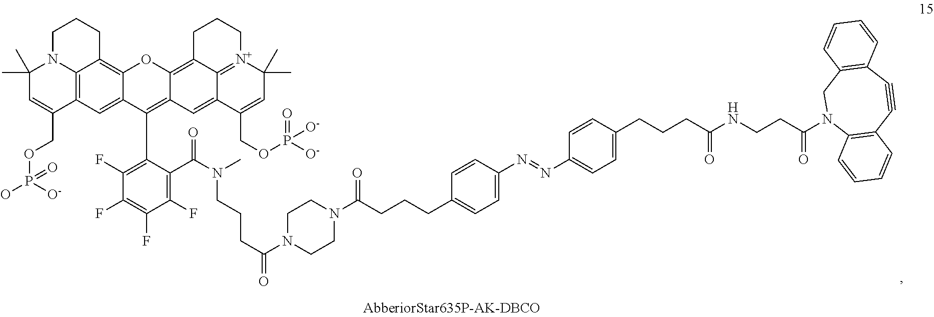 [SMILES: CN(CCCC(=O)N1CCN(C(=O)CCCc2ccc(/N=N/c3ccc(CCCC(=O)NCCC(=O)N4Cc5ccccc5C#Cc5ccccc54)cc3)cc2)CC1)C(=O)c1c(F)c(F)c(F)c(F)c1C1=c2cc3c4c(c2Oc2c1cc1c5c2CCCN5C(C)(C)C=C1COP(=O)([O-])O)CCC[N+]=4C(C)(C)C=C3COP(=O)([O-])[O-]]

15 was synthesized according to GP-A, using:
as fluorophore NHS ester: commercial “AbberiorStar635P NHS ester” (1.0 mg, 0.97 μmol, 1 eq.), and
as DBCO-azoaryl-piperazine: 5 (50 μL, 0.03 M in DMF, 1.5 μmol, 1.5 eq.)

HRMS (ESI): m/z Cs₅H₈₈F₄N₉O₁₄P2+[M+4H]⁺: calc.: 1596.5857, found: 1596.5883.

### 2-((1 E,3E)-5-((Z)-1-(6-(4-(2-(4-((4-(4-((3-(11,12-didehydrodibenzo[b,f]azocin-5(6H)-yl)-3-oxopropyl)amino)-4-oxobutoxy)phenyl)diazenyl)phenoxy)acetyl)piperazin-1-yl)-6-oxohexyl)-3,3-dimethylindolin-2-ylidene)penta-1,3-dien-1-yl)-1-ethyl-3,3-dimethyl-3H-indol-1-ium (16, Cy5-AO-DBCO)

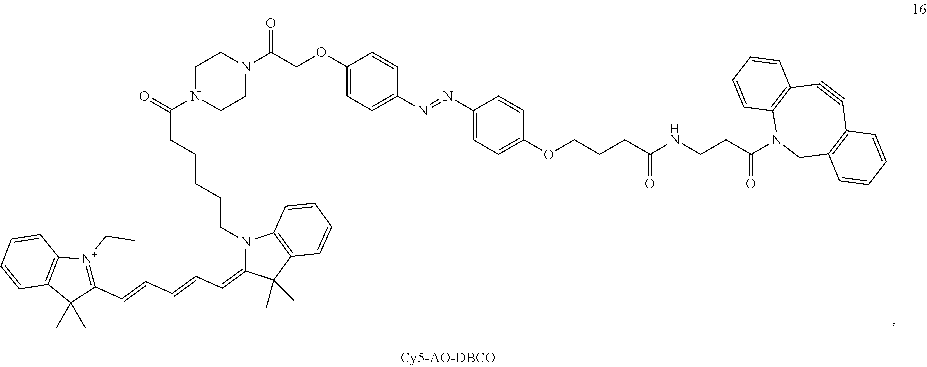 [SMILES: CC[N+]1=C(/C=C/C=C/C=C2\N(CCCCCC(=O)N3CCN(C(=O)COc4ccc(/N=N/c5ccc(OCCCC(=O)NCCC(=O)N6Cc7ccccc7C#Cc7ccccc76)cc5)cc4)CC3)c3ccccc3C2(C)C)C(C)(C)c2ccccc21]

16 was synthesized according to GP-B, using:
as fluorophore acid: Cy5 acid (1.5 mg, 3.02 μmol, 1 eq.), and
as DBCO-azoaryl-piperazine: 10 (83 μL, 0.03 M in DMF, 2.4 μmol, 0.8 eq.)

HRMS (ESI): m/z C₇₃H₇₉N₈O₆⁺ [M]⁺: calc.: 1163.61171, found: 1163.61310.

### N-(3-(11,12-didehydrodibenzo[b,f]azocin-5(6H)-yl)-3-oxopropyl)-4-(4-((4-(2-(4-Atto542-piperazin-1-yl)-2-oxoethoxy)phenyl)diazenyl)phenoxy)butanamide (17, Atto542-AO-DBCO)

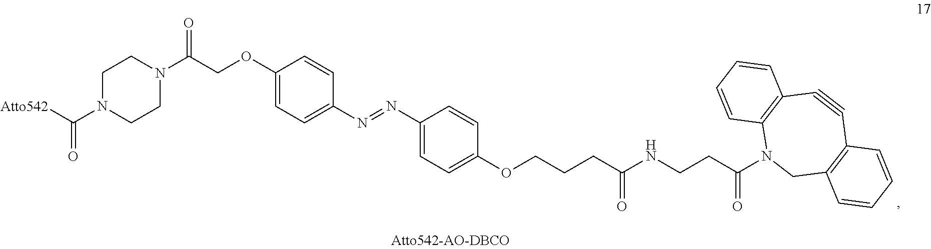

17 was synthesized according to GP-A, using:
as fluorophore NHS ester: Atto542 NHS ester (1.0 mg, 0.89 μmol, 1 eq.), and
as DBCO-azoaryl-piperazine: 10 (34 μL, 0.03 M in DMF, 0.98 μmol, 1.1 eq.).

HRMS (ESI): using the quoted Atto542 mass after coupling, expected m/z [M]⁻ is calc.: ˜1578, found: 1578.4958.

### N-(3-(11,12-didehydrodibenzo[b,f]azocin-5(6H)-yl)-3-oxopropyl)-4-(4-((4-(2-(4-Cy3B-piperazin-1-yl)-2-oxoethoxy)phenyl)diazenyl)phenoxy)butanamide (18, Cy3B-AO-DBCO)

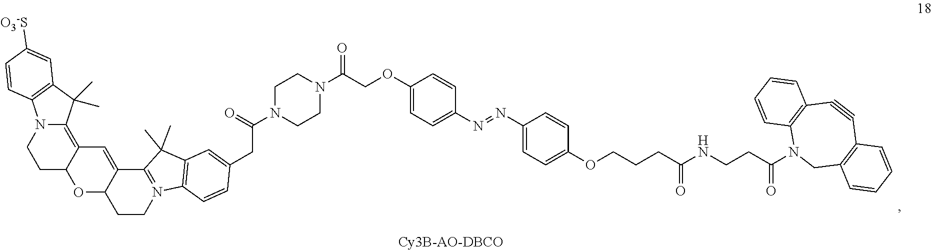

18 was synthesized according to GP-A, using:
as fluorophore NHS ester: Cy3B NHS ester (1.0 mg, 1.5 μmol, 1 eq.), and
as DBCO-azoaryl-piperazine: 10 (85 μL, 0.03 M in DMF, 1.7 μmol, 1.1 eq.).

HRMS (ESI): m/z C₇₁H₇₂N₈O₁₀S²⁺[M+2H]²⁺: calc.: 614.25406, found: 614.2547.

### 2-((1 E,3E)-5-((Z)-3-(5-(4-(2-(4-((4-(4-((3-(11,12-didehydrodibenzo[b,f]azocin-5(6H)-yl)-3-oxopropyl)amino)-4-oxobutoxy)phenyl)diazenyl)phenoxy)acetyl)piperazin-1-yl)-5-oxopentyl)-3-methyl-5-sulfonato-1-(3-sulfonatopropyl)indolin-2-ylidene)penta-1,3-dien-1-yl)-3,3-dimethyl-1-(3-sulfonatopropyl)-3H-indol-1-ium-5-sulfonate (19, AF647-AO-DBCO)

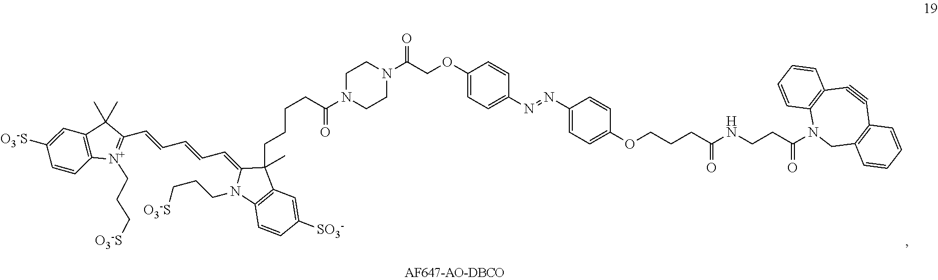

19 was synthesized according to GP-A, using:
as fluorophore NHS ester: AlexaFluor647 NHS ester (1.0 mg, 0.95 μmol, 1 eq.), and
as DBCO-azoaryl-piperazine: 10 (36 μL, 0.03 M in DMF, 1.0 μmol, 1.1 eq.).

HRMS (ESI): m/z C₇₅H₇₉NO₁₈S₄³⁻ [M]³⁻: calc.: 502.48039, found: 502.48034.

### N-(3-(11,12-didehydrodibenzo[b,f]azocin-5(6H)-yl)-3-oxopropyl)-4-(4-((4-(2-(4-AbberiorStar635P-piperazin-1-yl)-2-oxoethoxy)phenyl)diazenyl)phenoxy)butanamide (20, AbberiorStar635P-AO-DBCO)

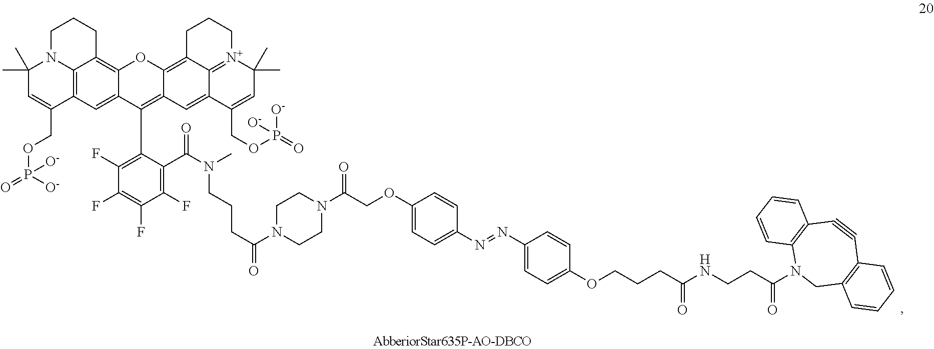 [SMILES: CN(CCCC(=O)N1CCN(C(=O)COc2ccc(/N=N/c3ccc(OCCCC(=O)NCCC(=O)N4Cc5ccccc5C#Cc5ccccc54)cc3)cc2)CC1)C(=O)c1c(F)c(F)c(F)c(F)c1C1=c2cc3c4c(c2Oc2c1cc1c5c2CCCN5C(C)(C)C=C1COP(=O)([O-])[O-])CCC[N+]=4C(C)(C)C=C3COP(=O)([O-])[O-]]

20 was synthesized according to GP-A, using:
as fluorophore NHS ester: AbberiorStar635P NHS ester (1.0 mg, 0.97 μmol, 1 eq.), and
as DBCO-azoaryl-piperazine: 10 (37 μL, 0.03 M in DMF, 1.1 μmol, 1.1 eq.).

HRMS (ESI): m/z C₈₃H₈₄F₄N₉O₁₆P₂⁺ [M+4H]⁺: calc.: 1600.5442, found: 1600.5796.

### 4.4 Synthesis of Labelled DNA Oligos from DBCO-Bearing Compounds or NHS Esters

### 4.4.1 General

Unless otherwise noted, oligo synthesis, dye coupling, purification, quality control and analysis were performed by commercial service provider Ella Biotech GmbH (Fuerstenfeldbruck, Germany) according to standard procedures.

DNA oligos for labelling were synthesised with two sequences: a 21-nt “permanent sequence” (5′-TATGAGAAGTTAGGAATGTTA-3′), and a 8-nt “DNA-PAINT sequence” (5′-GGAATGTT-3′); and they were synthesised to feature reactive handles including: 3′-amine, 3′-azide, 5′-trans-cyclooctene [TCO, from Biomers.net GmbH](calc: 6867 g/mol, found: 6869).

Complementary biotinylated 21-nt DNA-sequences (5′-biotin-TAACATTCCTAACTTCTCATA-3′ and 5′-TAACATTCCTAACTTCTCATA-biotin-3′) were synthesised similarly, and used for hybridization as described below.

### 4.4.2 Labelled DNA Oligos from DBCO-Bearing Compounds of the Invention

The azide oligos were coupled to the fluorophore-azoaryl-DBCO conjugates by copper-free click chemistry.

Analysis for 21-Nt Oligos Bearing Compounds of the Invention, from DBCO Coupling:

Cy5-AK-21nt: MW: 8192; ESI-MS (found): m/z=743.7 (M¹¹⁻); ESI-MS (deconvoluted): 8191.3 Da. Atto542-AK-21 nt: MW: 8608; ESI-MS (found): m/z=781.6 (M¹¹⁻); ESI-MS (deconvoluted): 8608.6 Da. Cy3B-AK-21nt: MW: 8255; ESI-MS (found): m/z=824.6 (M¹⁰⁻); ESI-MS (deconvoluted): 8255.6 Da. AlexaFluor647-AK-21 nt: MW: 8540; ESI-MS (found): m/z=609.0 (M¹⁴⁻); ESI-MS (deconvoluted): 8539.8 Da. AbberiorStar635P-AK-21nt: MW: 8625; ESI-MS (found): m/z=861.8 (M¹⁰⁻); ESI-MS (deconvoluted): 8628.1 Da. Cy5-AO-21nt: MW: 8196; ESI-MS (found): m/z=818.5 (M¹⁰⁻); ESI-MS (deconvoluted): 8195.0 Da. Atto542-AO-21nt: MW: 8612; ESI-MS (found): m/z=781.9 (M¹¹⁻); ESI-MS (deconvoluted): 8612.4 Da. Cy3B-AO-21nt: MW: 8259; ESI-MS (found): m/z=825.0 (M¹⁰⁻); ESI-MS (deconvoluted): 8258.9 Da. AlexaFluor647-AO-21nt: MW: 8544; ESI-MS (found): m/z=710.9 (M¹²⁻); ESI-MS (deconvoluted): 8543.0 Da. AbberiorStar635P-AO-21nt: MW: 8629; ESI-MS (found): m/z=862.2 (M¹⁰⁻); ESI-MS (deconvoluted): 8632.1 Da.

These 21-nt oligo conjugates of the self-healing DBCO dyes 11-20 are also referred to as DNA strands 11 b-20b, respectively.

Analysis for 8-Nt Oligos Bearing Compounds of the Invention, from DBCO Coupling:

Cy5-AK-8 nt: MW: 4109; ESI-MS (found): m/z=820.4 (M⁵⁻); ESI-MS (deconvoluted): 4107.1 Da. Atto542-AK-8 nt: MW: 4525; ESI-MS (found): m/z=753.1 (M⁶⁻); ESI-MS (deconvoluted): 4524.4 Da. Cy3B-AK-8 nt: MW: 4172; ESI-MS (found): m/z=833.3 (M⁵⁻); ESI-MS (deconvoluted): 4171.4 Da. AlexaFluor647-AK-8 nt: MW: 4454; ESI-MS (found): m/z=741.6 (M⁶⁻); ESI-MS (deconvoluted): 4454.4 Da. AbberiorStar635P-AK-8 nt: MW: 4542; ESI-MS (found): m/z=907.9 (M⁵⁻); ESI-MS (deconvoluted): 4544.4 Da. Cy5-AO-8 nt: MW: 4113; ESI-MS (found): m/z=821.3 (M⁵⁻); ESI-MS (deconvoluted): 4111.4 Da. Atto542-AO-8 nt: MW: 4529; ESI-MS (found): m/z=646.0 (M⁷); ESI-MS (deconvoluted): 4528.8 Da. Cy3B-AO-8 nt: MW: 4176; ESI-MS (found): m/z=834.1 (M⁵⁻); ESI-MS (deconvoluted): 4175.3 Da. AlexaFluor647-AO-8 nt: MW: 4458; ESI-MS (found): m/z=636.1 (M⁷); ESI-MS (deconvoluted): 4459.8 Da. AbberiorStar635P-AO-8 nt: MW: 4546; ESI-MS (found): m/z=908.7 (M⁵⁻); ESI-MS (deconvoluted): 4548.5 Da.

These 8-nt oligo conjugates of the self-healing DBCO dyes 11-20 are also referred to as DNA strands 11c-20c, respectively.

### 4.4.3 Reference (“Comparator”) Fluorescent DNA Oligos

Reference (“comparator”) fluorescent DNA oligos were either synthesised by Ella Biotech (Ref1=AbberiorStar635P-21 nt) or Eurofins (Ref2=AlexaFluor647-21 nt, Ref3=Cy5-21 nt, Ref4=Cy3B-21 nt, Ref5=Atto488-21nt, Ref6=Atto647N-21 nt, Ref7=Atto542-21nt) by coupling 3′-amine oligos to commercial fluorophore NHS esters, then purifying by HPLC (thus, e.g. Ref3 has the sequence 5′-TATGAGAAGTTAGGAATGTTA-Cy5-3′). Similarly, 5′-dye-labelled reference oligos were sourced from Eurofins (Ref8=Cy3-21nt; sequence 5′-Cy3-TATGAGAAGTTAGGAATGTTA-3′). Analytical data for all commercially sourced comparator oligos confirmed sequence identity (e.g. Ref1: MW: 7835; ESI-MS (found): m/z=869.8 (M⁹⁻); ESI-MS (deconvoluted): 7838.1 Da; a match of expected and deconvoluted molecular weight (MW) is hereafter denoted as “found”; Ref2: MW: 7567.3, “found”; Ref3: MW: 7189.0, “found”; Ref4: MW: 7268.3, “found”; Ref5: MW: 7297.3, “found”; Ref6: 7353.3, “found”; Ref8: 7055.3, “found”).

### 4.4.4 Labelled DNA Oligos from Tetrazine-Bearing Compounds of the Invention

Tetrazine-bearing compounds (conjugates of Cy3 or Cy5 with AO, AK, or AN: i.e. molecules 35a, 36a, 37a, 38a, 39a, 43a) were coupled to 5′-trans-cyclooctene (TCO) 21-nt DNA oligos (5′-TCO-TATGAGAAGTTAGGAATGTTA-3′) at 1:1 ratio at 0.5 mM concentration overnight at room temperature under stirring. Without further purification, the coupled product strands (respectively 35b, 36b, 37b, 38b, 39b, 43b) were then hybridized to a complementary 5′-biotin-labelled oligonucleotide (5′-biotin-TAACATTCCTAACTTCTCATA-3′). The unpurified product mixtures containing the target labelled and biotinylated dsDNA were then applied to neutravidin-decorated surfaces prepared as detailed in section 6, for imaging. This is unproblematic since unlabeled oligonucleotides are not observed in single molecule studies, and extensive washing steps are implemented before imaging to ensure that only specifically immobilized DNA duplexes are studied.

### 4.5 Synthesis of EY-AK for Fluorescence Correlation Spectroscopy

### 2,4,5,7-tetrabromo-6-hydroxy-9-(2-(piperazine-1-carbonyl)phenyl)-3H-xanthen-3-one (EY-P)

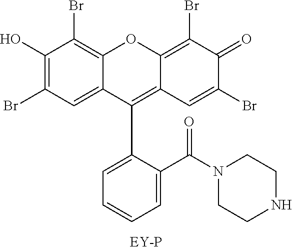 [SMILES: O=C(c1ccccc1-c1c2cc(Br)c(=O)c(Br)c-2oc2c(Br)c(O)c(Br)cc12)N1CCNCC1]

N,N′-dicyclohexylcarbodiimide (631 mg, 3.06 mmol, 1.54 eq.) and N-hydroxysuccinimide (305 mg, 2.65 mmol, 1.3 eq.) were added to a solution of commercial eosin Y disodium salt (disodium 2-(6-oxido-3-oxo-3H-xanthen-9-yl)benzoate) (1372 mg, 1.98 mmol, 1 eq.) in anhydrous DMF (6 mL) under nitrogen atmosphere and heated to 80° C. for 1 h. The reaction mixture was allowed to cool to room temperature. Piperazine (372 mg, 4.32 mmol, 2.2 eq.) and TEA (0.90 mL, 648 mg, 6.40 mmol, 3.2 eq.) were added and the mixture was stirred at room temperature for 16 h. The reaction mixture was concentrated under reduced pressure and purified by silica gel column chromatography (20-33% MeOH in DCM) to yield EY-P (800 mg, 1.12 mmol, 56%,) as a red solid.

¹H-NMR (500 MHz; dimethylsulfoxide-d₆): δ=7.74-7.68 (m, 2H), 7.70-7.64 (m, 1H), 7.54-7.47 (m, 1H), 7.05 (s, 2H), 3.50-3.43 (m, 4H), 2.94-2.83 (m, 4H).

¹³C-NMR (126 MHz; dimethylsulfoxide-d₆): δ=172.8, 168.4, 166.5, 152.8, 148.4, 134.7, 131.1, 130.5, 129.8, 129.6, 127.3, 118.4, 109.5, 99.5, 44.8, 43.3.

HR-MS (ESI): m/z C₂₄H₁₇Br₄N₂O₄⁺[M+H]⁺: calc.: 716.78754, found: 716.78786.

### 2,4,5,7-tetrabromo-9-(2-(4-(4-(4-((4-butylphenyl)diazenyl)phenyl)butanoyl)piperazine-1-carbonyl)phenyl)-6-hydroxy-3H-xanthen-3-one (EY-AK)

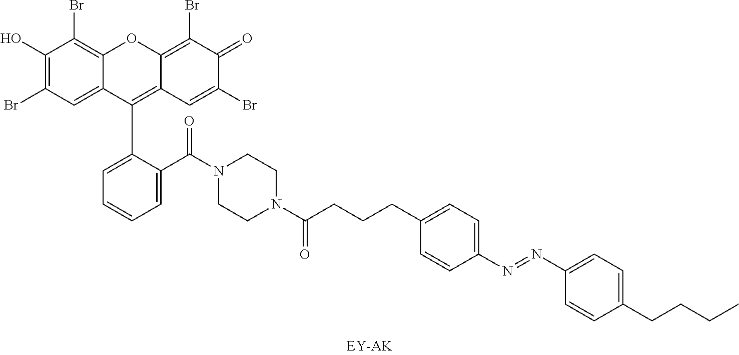 [SMILES: CCCCc1ccc(/N=N/c2ccc(CCCC(=O)N3CCN(C(=O)c4ccccc4-c4c5cc(Br)c(=O)c(Br)c-5oc5c(Br)c(O)c(Br)cc45)CC3)cc2)cc1]

4-(4-((4-butylphenyl)diazenyl)phenyl)butanoic acid (Frank; Nat Commun 2015 6, 7118) (25 mg, 77.1 μmol, 1 eq.), EYpip (55.2 mg, 77.1 μmol, 1 eq.), HBTU (35 mg, 92.4 μmol, 1.2 eq.) and TEA (28 μL, 39 mg, 386 μmol, 5 eq.) were dissolved in DMF (2.5 mL). The reaction mixture was stirred at room temperature for 16 h, diluted with water (300 μL) and AcOH (125 μL) and purified by reversed phase preparative HPLC to yield EY-AK (30 mg, 29.3 μmol, 38%) as an orange solid. Typical gradient used for preparative HPLC: 9:1 water (0.1% formic acid)/MeCN (0.1% formic acid) to MeCN (0.1% formic acid) over 27 min.

¹H-NMR (500 MHz; dimethylsulfoxide-d₆): δ=7.83-7.76 (m, 4H), 7.73-7.67 (m, 2H), 7.63 (s, 1H), 7.52-7.47 (m, 1H), 7.39 (dd, J=8.7, 6.7 Hz, 4H), 7.04 (d, J=8.1 Hz, 2H), 3.28 (s, 4H), 2.71-2.62 (m, 4H), 2.38-2.28 (m, 2H), 1.85-1.78 (m, 1H), 1.65-1.55 (m, 2H), 1.33 (h, J=7.4 Hz, 2H), 0.91 (t, J=7.4 Hz, 3H).

¹³C-NMR (126 MHz; dimethylsulfoxide-d₆): δ=170.4, 168.3, 166.6, 166.5, 162.5, 154.4, 152.8, 150.3, 150.3, 148.7, 146.2, 145.8, 135.0, 129.7, 129.3, 129.3, 127.3, 122.6, 122.5, 120.1, 118.3, 109.6, 99.5, 46.8, 41.1, 34.7, 34.4, 32.9, 31.5, 26.2, 21.8, 13.8.

HR-MS (ESI): m/z C₄₄H₃₉Br₄N₄O₅⁺ [M+H]⁺: calc.: 1022.96075; found: 1022.96134.

### 4.6 Synthesis of Molecules of the Invention with Tetrazine Tether Units

### 4.6.1 Synthesis of Monovalent Azobenzenes

### 4-(4-((4-butylphenyl)diazenyl)phenyl)butanoic acid (21, AK)

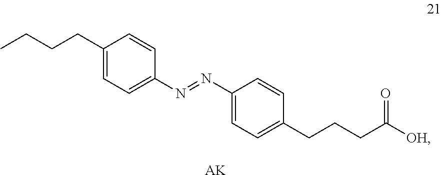 [SMILES: CCCCc1ccc(/N=N/c2ccc(CCCC(=O)O)cc2)cc1 ; "AK"]

Adapting a known procedure,^{[1]} a solution of Oxone® (3.67 g, 11.9 mmol, 9.0 eq.) in water (24 mL) was added to a solution of methyl 4-butylaniline (0.30 g, 2.0 mmol, 1.5 eq.) in DCM (24 mL, 0.08 M), and the biphasic reaction mixture was vigorously stirred at room temperature for 15 h. The phases were separated, the aqueous phase was extracted with DCM (2×50 mL) and the combined organic phases were washed with an aqueous hydrochloric acid solution (1 M, 50 mL), a saturated aqueous NaHCO₃ solution (50 mL), water (50 mL) and a saturated aqueous NaCl solution (50 mL). 4-(4-aminophenyl)butanoic acid (0.25 g, 1.3 mmol, 1.0 eq.) and AcOH (100%, 18 mL) were added to the organic phase and the reaction mixture was stirred at room temperature for 15 min. The DCM in reaction mixture was removed under reduced pressure and stirring was continued for additional 6 h at room temperature. The mixture was then concentrated under reduced pressure, co-evaporated with toluene (3×5 mL) and purified by silica gel flash column chromatography (DCM/MeOH/AcOH: 96.5/3.0/0.5) to yield 21 (0.23 g, 0.72 mmol, 54%) as a yellow/orange solid.

LRMS (ESI): m/z C₂₀H₂₅N₂O₂⁺[M+H]⁺: calc.: 325.19105, found: 325.2.

¹H-NMR (400 MHz, chloroform-d₁) δ (ppm): 7.86-7.79 (m, 4H), 7.35-7.29 (m, 4H), 2.81-2.65 (m, 4H), 2.41 (t, J=7.4, 2H), 2.08-1.96 (m, 2H), 1.71-1.59 (m, 2H), 1.45-1.31 (m, 2H), 0.94 (t, J=7.3, 3H).

¹³C-NMR (101 MHz, chloroform-d₁) δ (ppm): 177.4, 151.4, 151.1, 146.6, 144.5, 129.3, 129.3, 123.1, 122.9, 35.7, 35.0, 33.6, 33.0, 26.2, 22.5, 14.1.

### Mixture of methyl (22a) & ethyl (22b) 4-(4-((4-hydroxyphenyl)diazenyl)phenoxy)butanoate esters

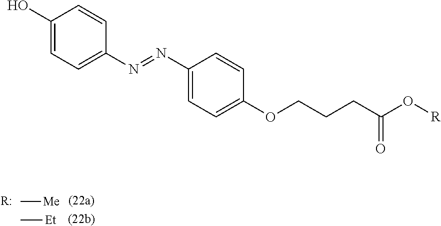

A solution of hydrochloric acid in dioxane (4 M, 6.0 mL, 5 eq.) was added to a solution of ethyl 4-(4-((tert-butoxycarbonyl)amino)phenoxy)butanoate^{[2]} (1.55 g, 4.8 mmol, 1 eq.) in hexane (50 mL, 0.1 M) and the reaction mixture was stirred at room temperature for 1 h. The precipitate was filtrated off and washed with hexane (2×5 mL). The crude ethyl 4-(4-aminophenoxy)butanoate hydrochloride (0.72 g, 2.77 mmol, 58%) was obtained as a white solid and was used directly without further purification, being resuspended in MeOH (20 mL, 0.14 M) and a solution of hydrochloric acid solution in dioxane (4 M, 4.16 mL, 6 eq.) was added. The mixture was cooled down to 0° C., a solution of isopentyl nitrite (0.41 mL, 3.05 mmol, 1.1 eq.) in MeOH (5 mL, 0.61 M) was added dropwise and the resulting reaction mixture was stirred for 30 min at 0° C. A solution of phenol (0.29 g, 3.05 mmol, 1.1 eq.) in MeOH (5 mL, 0.61 M) cooled to 0° C. was then added to the reaction mixture. After stirring for 5 min, the pH of the reaction mixture was adjusted to pH=11-12 with an aqueous NaOH solution (2 M) and stirring was continued for additional 30 min at 0° C. The reaction mixture was then neutralized with a saturated aqueous NH₄Cl solution, extracted with DCM (3×50 mL) and dried with a saturated aqueous NaCl solution (50 mL). The organic phase was concentrated under reduced pressure and purified by silica gel flash column chromatography (DCM/MeOH: 100/0 to 94/6) to yield a 1/1 mixture of 22a & 22b (0.33 g, 1.03 mmol, 36%) as a red solid. The 1/1 mixture of 22a & 22b was thus obtained from 4-(4-((tert-butoxycarbonyl)amino)phenoxy)butanoate with 21% yield over 2 steps.

22a:

LRMS (ESI): m/z C₁₇H₁₉N₂O₄⁺[M+H]⁺: calc. 315.13393, found: 315.2.

¹H-NMR (400 MHz, chloroform-d₁) δ (ppm): 7.89-7.79 (m, 4H), 7.01-6.89 (m, 4H), 4.09 (t, J=6.1, 2H), 3.71 (s, 3H), 2.56 (m, 2H), 2.20-2.12 (m, 2H).

22b:

LRMS (ESI): m/z C₁₈H₂₁N₂O₄⁺[M+H]⁺: calc.: 329.14958, found: 329.3.

¹H-NMR (400 MHz, chloroform-d₁) δ (ppm): 7.89-7.79 (m, 4H), 7.01-6.89 (m, 4H), 4.16 (q, J=7.2, 2H), 4.09 (t, J=6.1, 2H), 2.56 (m, 2H), 2.20-2.12 (m, 2H), 1.27 (t, J=7.2, 3H).

### Mixture of methyl (23a) & ethyl (23b) 4-(4-((4-butoxyphenyl)diazenyl)phenoxy)butanoate

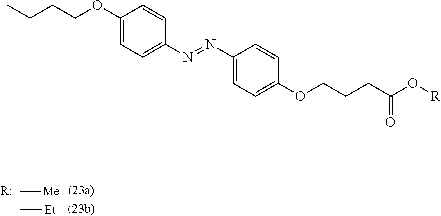

A pressure tube was charged with a solution of a 1/1 mixture of 22a & 22b (300 mg, 0.93 mmol, 1.0 eq.) and K₂CO₃ (514 mg, 3.72 mmol, 4.0 eq.) in acetone (9 mL, 0.1 M). 1-Bromobutane (200 μL, 1.86 mmol, 2.0 eq.) was added and reaction mixture was stirred at 65° C. for 16 h. The formed precipitate was filtrated off and washed with acetone (2×5 mL). The crude product was purified by silica gel flash column chromatography (hexanes/EtOAc: 95/5 to 70/30) to yield a 1/1 mixture of 23a & 23b (184 mg, 0.49 mmol, 52%) as an orange solid.

23a:

HRMS (ESI): m/z C₂₁H₂₇N₂O₄⁺[M+H]⁺: calc.: 371.19653, found: 371,19636.

¹H-NMR (400 MHz, chloroform-d₁) δ (ppm): 7.90-7.82 (m, 4H), 7.03-6.94 (m, 4H), 4.09 (t, J=6.1, 2H), 4.04 (t, J=6.5, 2H), 3.70 (s, 3H), 2.55 (q, J=7.3, 2H), 2.21-2.09 (m, 2H), 1.86-1.75 (m, 2H), 1.58-1.46 (m, 2H), 0.99 (t, J=7.4, 3H).

23b:

HRMS (ESI): m/z C₂₂H₂₉N₂O₄⁺[M+H]⁺: calc.: 385.21218, found: 385.21198.

¹H-NMR (400 MHz, chloroform-d₁) δ (ppm): 7.90-7.82 (m, 4H), 7.03-6.94 (m, 4H), 4.16 (q, J=7.1, 2H), 4.09 (t, J=6.1, 2H), 4.04 (t, J=6.5, 2H), 2.55 (q, J=7.3, 2H), 2.21-2.09 (m, 2H), 1.86-1.75 (m, 2H), 1.59-1.45 (m, 2H), 1.27 (t, J=7.1, 3H), 0.99 (t, J=7.4, 3H).

### 4-(4-((4-butoxyphenyl)diazenyl)phenoxy)butanoic acid (24, AO)

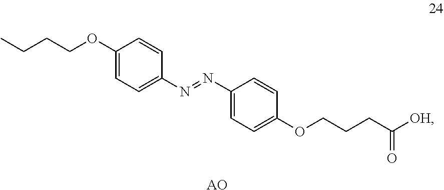 [SMILES: CCCCOc1ccc(/N=N/c2ccc(OCCCC(=O)O)cc2)cc1 ; "AO"]

An aqueous solution of LiOH (1 mL, 2 M,) was added to a solution of 1/1 mixture of 23a & 23b (184 mg, 0.49 mmol, 1.0 eq.) in MeCN/MeOH (1/1, 2 mL, 0.24 M) and the reaction mixture was stirred at room temperature for 22 h. The mixture was then concentrated under reduced pressure, acidified with an excess of AcOH and extracted with DCM (3×5 mL). The combined organic phases were dried (Na₂SO₄), concentrated under reduced pressure and purified by silica gel flash column chromatography (DCM/MeOH: 100/0 to 90/10) to yield 24 (AO, 161 mg, 0.45 mmol, 92%) as a yellow solid.

HRMS (ESI): m/z C₂₀H₂₅N₂O₄⁺[M+H]⁺: calc.: 357.18088, found: 357.18071.

¹H-NMR (400 MHz, chloroform-d₁) δ (ppm): 7.85-7.77 (m, 4H), 6.97-6.92 (m, 4H), 4.05 (t, J=6.1, 2H), 4.00 (t, J=6.5, 2H), 2.51 (t, J=7.3, 2H), 2.15-2.04 (m, 2H), 1.76 (dq, J=8.7, 6.6, 2H), 1.54-1.41 (m, 2H), 0.95 (t, J=7.4, 3H).

¹³C-NMR (101 MHz, chloroform-d₁) δ (ppm): 175.8, 161.3, 160.9, 147.1, 146.9, 124.4, 124.3, 114.8, 114.7, 68.1, 67.1, 31.3, 30.4, 24.6, 19.3, 13.9.

### 5-((4-((4-acetamidophenyl)diazenyl)phenyl)amino)-5-oxopentanoic acid (25, AN)

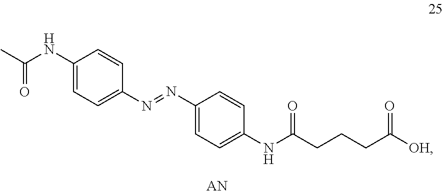 [SMILES: CC(=O)Nc1ccc(/N=N/c2ccc(NC(=O)CCCC(=O)O)cc2)cc1]

A pressure tube was charged with a solution of N-(4-((4-aminophenyl)diazenyl)phenyl)-acetamide^{[3]} (50 mg, 0.20 mmol, 1.0 eq.) in acetone (2 mL, 0.1 M). Glutaric anhydride (112 mg, 0.98 mmol, 5.0 eq.) and pyridine (158 μL, 1.97 mmol, 10.0 eq.) were added and the reaction mixture was stirred at 70° C. for 3 h. The formed precipitate was filtrated off and washed with Et₂O (5 mL). 25 (AN, 28 mg, 0.48 mmol, 39%) was obtained as an orange solid.

HRMS (ESI): m/z C₁₈H₂₁N₄O₄⁺ [M+H]⁺: calc.: 369.15573, found: 369.15584.

¹H-NMR (400 MHz, dimethyl sulfoxide-d₆) δ (ppm): 12.12 (s, 1H), 10.28 (s, 1H), 10.25 (s, 1H), 7.86-7.75 (m, 8H), 2.41 (t, J=7.4, 2H), 2.29 (t, J=7.3, 2H), 2.09 (s, 3H), 1.82 (m, 2H).

¹³C-NMR (101 MHz, dimethyl sulfoxide-d₆)) δ (ppm): 174.2, 171.2, 168.8, 147.51, 142.0, 141.9, 123.4, 119.2, 119.1, 35.5, 33.0, 24.2, 20.3.

### 4.6.2 Synthesis of Azobenzene-Tetrazine-Lysine Conjugates

### N₂-(tert-butoxycarbonyl)-N-(4-(4-((4-butylphenyl)diazenyl)phenyl)butanoyl)lysine (26)

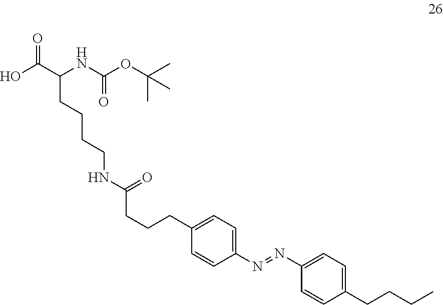 [SMILES: CCCCc1ccc(/N=N/c2ccc(CCCC(=O)NCCCCC(NC(=O)OC(C)(C)C)C(=O)O)cc2)cc1]

26 was synthesized according to GP-C1, using:
as azobenzene carboxylic acid: 21 (AK, 50 mg, 0.15 mmol, 1.00 eq., 0.25 M in DMF)
as incubation time after the addition of HBTU (0.95 eq.) and DIPEA (5.00 eq.): 30 min
Na-(tert-Butoxycarbonyl)-L-lysine (42 mg, 0.17 mmol 1.10 eq., 0.28 M in DMF)
silica gel flash column chromatography: DCM/MeOH 100/0 to 90/10

26 (51.8 mg, 0.09 mmol, 61%) was obtained as a yellow solid.

HRMS (ESI): m/z C₃₁H₄₄N₄NaO₅⁺ [M+Na]⁺: calc.: 575.32039, found: 575.31943.

### tert-butyl (6-(4-(4-((4-butylphenyl)diazenyl)phenyl)butanamido)-1-((4-(6-methyl-1,2,4,5-tetrazin-3-yl)benzyl)amino)-1-oxohexan-2-yl)carbamate (27)

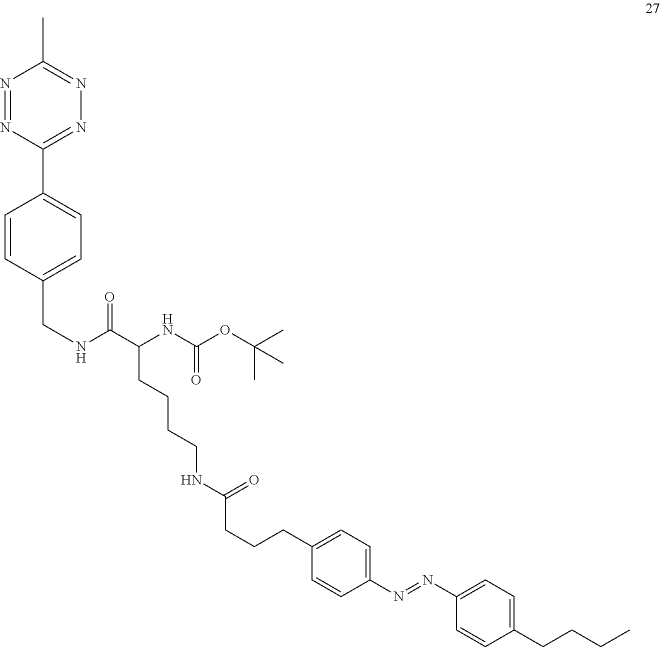 [SMILES: CCCCc1ccc(/N=N/c2ccc(CCCC(=O)NCCCCC(NC(=O)OC(C)(C)C)C(=O)NCc3ccc(-c4nnc(C)nn4)cc3)cc2)cc1]

27 was synthesized according to GP-C2, using:
as azobenzene-lysin carboxylic acid: 26 (21.9 mg, 39.6 μmol, 1.00 eq., 0.10 M in DMF)
as incubation time after the addition of HBTU (0.95 eq.) and DIPEA (5.00 eq.): 60 min
methyltetrazine-amine hydrochloride (8.5 mg, 35.6 μmol, 0.90 eq., neat)
silica gel flash column chromatography: DCM/MeOH 100/0 to 96/4

27 (17.8 mg, 24.2 μmol, 68%) was obtained as a red solid.

HRMS (ESI): m/z C₄₁H₅₃N₉O₄⁺[M+Na]⁺: calc.: 758.41127, found: 758.41055.

### 2-amino-6-(4-(4-((4-butylphenyl)diazenyl)phenyl)butanamido)-N-(4-(6-methyl-1,2,4,5-tetrazin-3-yl)benzyl)hexanamide (28, AK-(Tet-)Lys-NH₂)

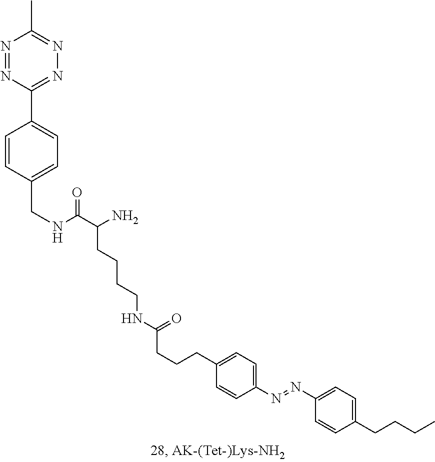 [SMILES: CCCCc1ccc(/N=N/c2ccc(CCCC(=O)NCCCCC(N)C(=O)NCc3ccc(-c4nnc(C)nn4)cc3)cc2)cc1]

28 was synthesized according to GP-C3, using:
as azobenzene-(tetrazine)-lysine tert-butyloxycarbonyl: 27 (17.8 mg, 24.2 μmol, 1.00 eq., 0.061 M in DMF)
as incubation time after the addition of trifluoroacetic acid (50 μL): 60 min

28 (AK-(Tet-)Lys-NH₂, 14.3 mg, 22.5 μmol, 93%) was obtained as a red resin.

LRMS (ESI): m/z C₃₆H₄₆N₉O₂⁺[M+H]⁺: calc.: 636.37690, found: 636.3.

### N²-(tert-butoxycarbonyl)-NM-(4-(4-((4-butoxyphenyl)diazenyl)phenoxy)butanoyl)lysine (29)

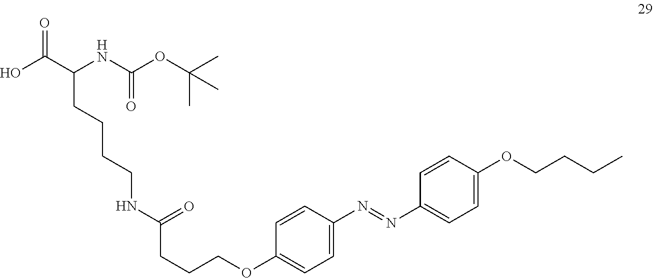 [SMILES: CCCCOc1ccc(/N=N/c2ccc(OCCCC(=O)NCCCCC(NC(=O)OC(C)(C)C)C(=O)O)cc2)cc1]

29 was synthesized according to GP-C1, using:
as azobenzene carboxylic acid: 24 (AO, 55 mg, 0.15 mmol, 1.00 eq., 0.25 M in DMF)
as incubation time after the addition of HBTU (0.95 eq.) and DIPEA (5.00 eq.): 30 min
Na-(tert-Butoxycarbonyl)-L-lysine (42 mg, 0.17 mmol 1.10 eq., 0.28 M in DMF)
silica gel flash column chromatography: DCM/MeOH 100/0 to 90/10

29 (34.8 mg, 0.06 mmol, 41%) was obtained as a yellow solid.

HRMS (ESI): m/z C₃₁H₄₄N₄NaO₇⁺[M+Na]⁺: calc.: 607.31002, found: 607.30939.

### tert-butyl (6-(4-(4-((4-butoxyphenyl)diazenyl)phenoxy)butanamido)-1-((4-(6-methyl-1,2,4,5-tetrazin-3-yl)benzyl)amino)-1-oxohexan-2-yl)carbamate (30)

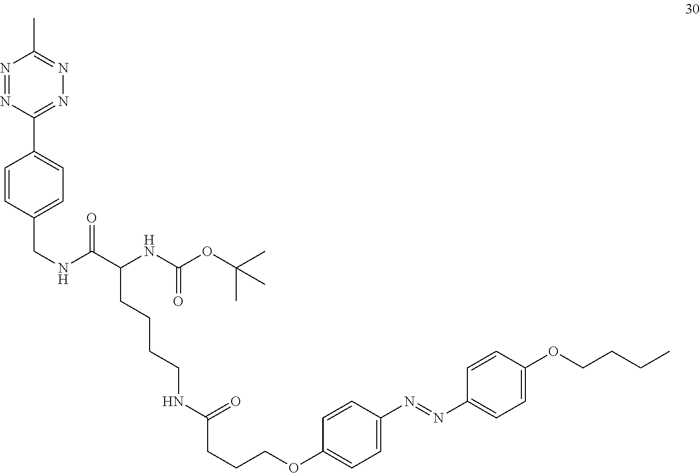 [SMILES: CCCCOc1ccc(/N=N/c2ccc(OCCCC(=O)NCCCCC(NC(=O)OC(C)(C)C)C(=O)NCc3ccc(-c4nnc(C)nn4)cc3)cc2)cc1]

30 was synthesized according to GP-C2, using:
as azobenzene-lysin carboxylic acid: 29 (23.1 mg, 39.6 μmol, 1.00 eq., 0.10 M in DMF)
as incubation time after the addition of HBTU (0.90 eq.) and DIPEA (5.00 eq.): 60 min
methyltetrazine-amine hydrochloride (8.5 mg, 35.6 μmol, 0.90 eq., neat)
silica gel flash column chromatography: DCM/MeOH 100/0 to 96/4

30 (21.2 mg, 27.6 μmol, 78%) was obtained as a red solid.

HRMS (ESI): m/z C₄₁H₅₃N₉NaO₆⁺ [M+Na]⁺: calc.: 790.40110, found: 790.40081.

### (E)-2-amino-6-(4-(4-((4-butoxyphenyl)diazenyl)phenoxy)butanamido)-N-(4-(6-methyl-1,2,4,5-tetrazin-3-yl)benzyl)hexanamide (31, AO-(Tet-)Lys-NH₂)

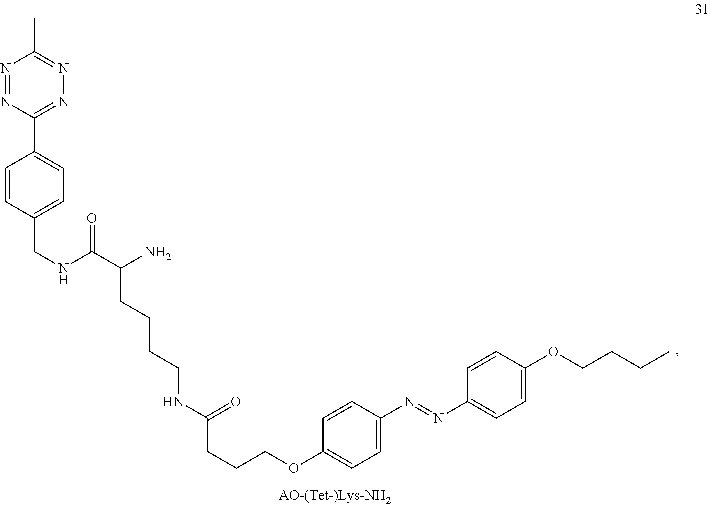 [SMILES: CCCCOc1ccc(/N=N/c2ccc(OCCCC(=O)NCCCCC(N)C(=O)NCc3ccc(-c4nnc(C)nn4)cc3)cc2)cc1]

31 was synthesized according to GP-C3, using:
as azobenzene-(tetrazine)-lysine tert-butyloxycarbonyl: 30 (21.1 mg, 27.5 μmol, 1.00 eq., 0.069 M in DMF)
as incubation time after the addition of trifluoroacetic acid (50 μL): 50 min
silica gel flash column chromatography: DCM/MeOH 100/0 to 85/15

31 (AO-(Tet-)Lys-NH₂, 16.9 mg, 25.3 μmol, 92%) was obtained as a red resin/solid.

HRMS (ESI): m/z C₃₆H₄₆N₉O₄⁺[M+H]⁺: calc.: 668.36673, found: 668.36621.

### N⁶-(5-((4-((4-acetamidophenyl)diazenyl)phenyl)amino)-5-oxopentanoyl)-1-(tert-butoxycarbonyl)lysine (32)

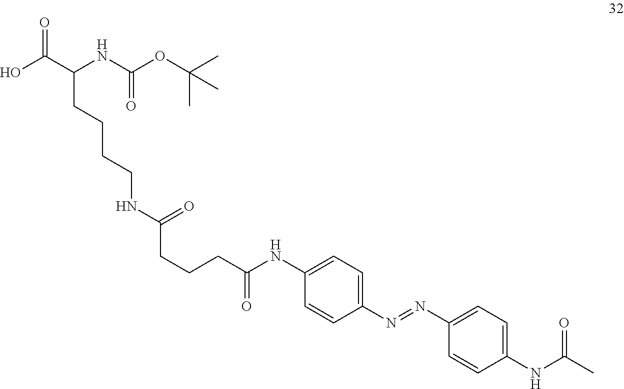 [SMILES: CC(=O)Nc1ccc(/N=N/c2ccc(NC(=O)CCCC(=O)NCCCCC(NC(=O)OC(C)(C)C)C(=O)O)cc2)cc1]

32 was synthesized according to GP-C1, using:
as azobenzene carboxylic acid: 25 (AN, 22 mg, 0.06 mmol, 1.00 eq., 0.10 M in DMF)
as incubation time after the addition of HBTU (0.95 eq.) and DIPEA (5.00 eq.): 10 min
Na-(tert-Butoxycarbonyl)-L-lysin (16 mg, 0.07 mmol 1.10 eq., 0.11 M in DMF)
silica gel flash column chromatography: DCM/MeOH 90/10 to 70/30

32 (20.7 mg, 0.03 mmol, 61%) was obtained as an orange solid.

HRMS (ESI): m/z C₃₀H₄₀N₆NaO₇⁺[M+Na]⁺: calc.: 619.28507, found: 619.28452.

### tert-butyl (6-(5-((4-((4-acetamidophenyl)diazenyl)phenyl)amino)-5-oxopentanamido)-1-((4-(6-methyl-1,2,4,5-tetrazin-3-yl)benzyl)amino)-1-oxohexan-2-yl)carbamate (33)

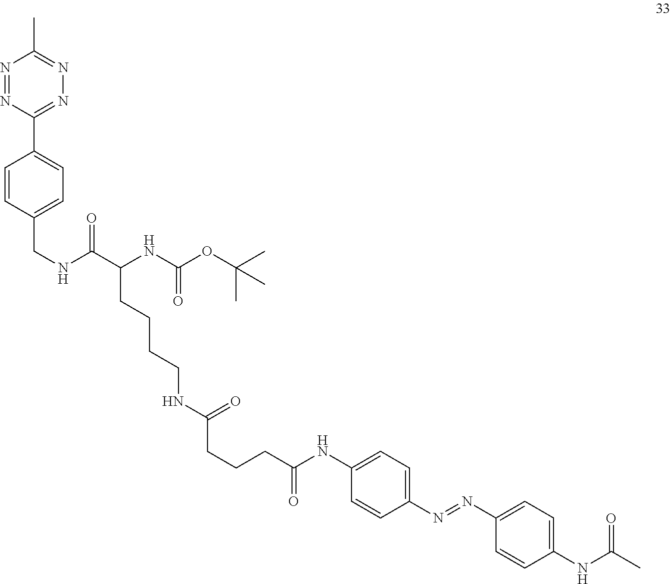 [SMILES: CC(=O)Nc1ccc(/N=N/c2ccc(NC(=O)CCCC(=O)NCCCCC(NC(=O)OC(C)(C)C)C(=O)NCc3ccc(-c4nnc(C)nn4)cc3)cc2)cc1]

33 was synthesized according to GP-C2, using:
as azobenzene-lysin carboxylic acid: 32 (19.3 mg, 32.3 μmol, 1.00 eq., 0.08 M in DMF)
as incubation time after the addition of HBTU (1.00 eq.) and DIPEA (7.00 eq.): 15 min
methyltetrazine-amine hydrochloride (7.7 mg, 32 μmol, 1.0 eq., 0.13 M in DMF)
silica gel flash column chromatography: DCM/MeOH (+0.1% NEt₃) 100/0 to 94/6

33 (15.1 mg, 19.4 μmol, 60%) was obtained as a red solid.

HRMS (ESI): m/z C₄₀H₄₉N₁₁NaO₆⁺ [M+Na]⁺: calc.: 802.37595, found: 802.37431.

### N¹-(4-((4-acetamidophenyl)diazenyl)phenyl)-A-(5-amino-6-((4-(6-methyl-1,2,4,5-tetrazin-3-yl)benzyl)amino)-6-oxohexyl)glutaramide (34a, AN-(Tet-)Lys-NH₂)

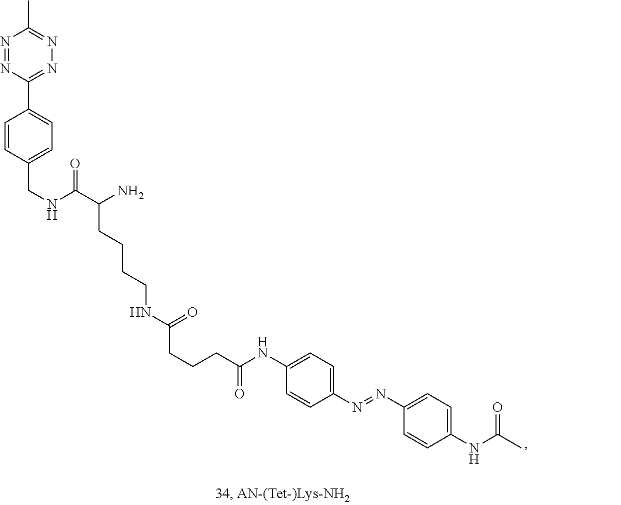 [SMILES: CC(=O)Nc1ccc(/N=N/c2ccc(NC(=O)CCCC(=O)NCCCCC(N)C(=O)NCc3ccc(-c4nnc(C)nn4)cc3)cc2)cc1]

34 was synthesized according to GP-C3, using:
as azobenzene-(tetrazine)-lysine tert-butyloxycarbonyl: 33 (15.1 mg, 19.4 μmol, 1.00 eq., 0.049 M in DMF)
as incubation time after the addition of trifluoroacetic acid (50 μL): 40 min

34 (AN-(Tet-)Lys-NH₂, 11.5 mg, 16.9 μmol, 87%) was obtained as a red solid.

HRMS (ESI): m/z C₃₅H₄₂N₁₁O₄⁺[M+H]⁺: calc.: 680.34158, found: 680.34036.

### 4.6.3 Synthesis of Azobenzene-(Tetrazine)Lysine-Fluorophore Molecules of the Invention

### 1-(6-((6-(4-(4-((4-butylphenyl)diazenyl)phenyl)butanamido)-1-((4-(6-methyl-1,2,4,5-tetrazin-3-yl)benzyl)amino)-1-oxohexan-2-yl)amino)-6-oxohexyl)-2-((E)-3-((E)-1-ethyl-3,3-dimethylindolin-2-ylidene)prop-1-en-1-yl)-3,3-dimethyl-3H-indol-1-ium (35a, AK-(Tet-)Lys-Cy3)

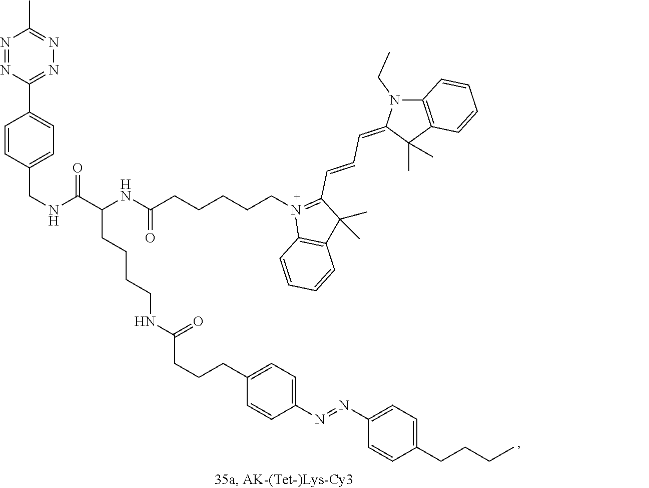

35a was synthesized according to GP-C4, using:
as fluorophore carboxylic acid: Cy3 carboxylic acid (3.03 mg, 6.42 μmol, 1.10 eq., 0.006 M in DMF)
as coupling reagent: TSTU (1.76 mg, 5.84 μmol, 1.00 eq., 17.4 mg/mL in DMF)
as C3: 28 (AK-(Tet-)Lys-NH₂, (3.53 mg, 5.55 μmol, 0.95 eq., in 100 μL DMF) incubation time after the addition of C3: 5 h

35a (AK-(Tet-)Lys-Cy3, 0.42 mg, 0.38 μmol, 7%) was obtained as a red/pink solid.

HRMS (ESI): m/z Cs₇H₈₂N₁₁O₃⁺[M]⁺: calc.: 1088.65966, found: 1088.65902.

### 1-(6-((6-(4-(4-((4-butoxyphenyl)diazenyl)phenoxy)butanamido)-1-((4-(6-methyl-1,2,4,5-tetrazin-3-yl)benzyl)amino)-1-oxohexan-2-yl)amino)-6-oxohexyl)-2-((E)-3-((E)-1-ethyl-3,3-dimethylindolin-2-ylidene)prop-1-en-1-yl)-3,3-dimethyl-3H-indol-1-ium (36a, AO-(Tet-)Lys-Cy3)

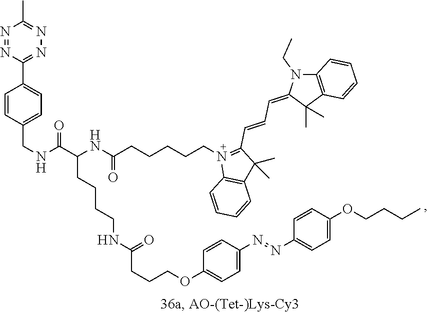

36a was synthesized according to GP-C4, using:
as fluorophore carboxylic acid: Cy3 carboxylic acid (1.65 mg, 3.49 μmol, 1.05 eq., 0.017 M in DMF)
as coupling reagent: HBTU (1.26 mg, 3.33 μmol, 1.00 eq., 12.6 mg/mL in DMF)
as C3: 31 (AO-(Tet-)Lys-NH₂, 2.00 mg, 2.99 μmol, 0.90 eq., in 100 μL DMF)
incubation time after the addition of C3: 4 h

36a (AO-(Tet-)Lys-Cy3, 0.42 mg, 0.38 μmol, 13%) was obtained as a red/pink solid.

HRMS (ESI): m/z C₆₇H₈₂N₁₁O₅ [M]: calc.: 1120.64949, found: 1120.64859.

### 1-(6-((6-(4-(4-((4-butoxyphenyl)diazenyl)phenoxy)butanamido)-1-((4-(6-methyl-1,2,4,5-tetrazin-3-yl)benzyl)amino)-1-oxohexan-2-yl)amino)-6-oxohexyl)-2-((1 E,3E)-5-((E)-1-ethyl-3,3-dimethylindolin-2-ylidene)penta-1,3-dien-1-yl)-3,3-dimethyl-3H-indol-1-ium (37a, AO-(Tet-)Lys-Cy5)

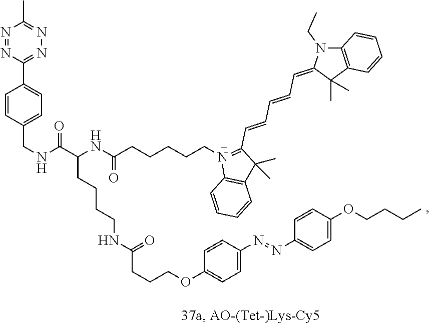

37a was synthesized according to GP-C4, using:
as fluorophore carboxylic acid: Cy5 carboxylic acid (1.74 mg, 3.49 μmol, 1.05 eq., 0.017 M in DMF)
as coupling reagent: HBTU (1.26 mg, 3.33 μmol, 1.00 eq., 12.6 mg/mL in DMF)
as C3: 31 (AO-(Tet-)Lys-NH₂, 2.00 mg, 2.99 μmol, 0.90 eq., in 100 μL DMF incubation time after the addition of C3: 1 h

37a (AO-(Tet-)Lys-Cy5, 0.48 mg, 4.18 μmol, 14%) was obtained as a blue/green solid.

HRMS (ESI): m/z C₆₉H₈₄N₁₁O₅ [M]⁺: calc.: 1146.66514, found: 1146.66425.

### 1-(6-((6-(5-((4-((4-acetamidophenyl)diazenyl)phenyl)amino)-5-oxopentanamido)-1-((4-(6-methyl-1,2,4,5-tetrazin-3-yl)benzyl)amino)-1-oxohexan-2-yl)amino)-6-oxohexyl)-2-((E)-3-((E)-1-ethyl-3,3-dimethylindolin-2-ylidene)prop-1-en-1-yl)-3,3-dimethyl-3H-indol-1-ium (38a, AN-(Tet-)Lys-Cy3)

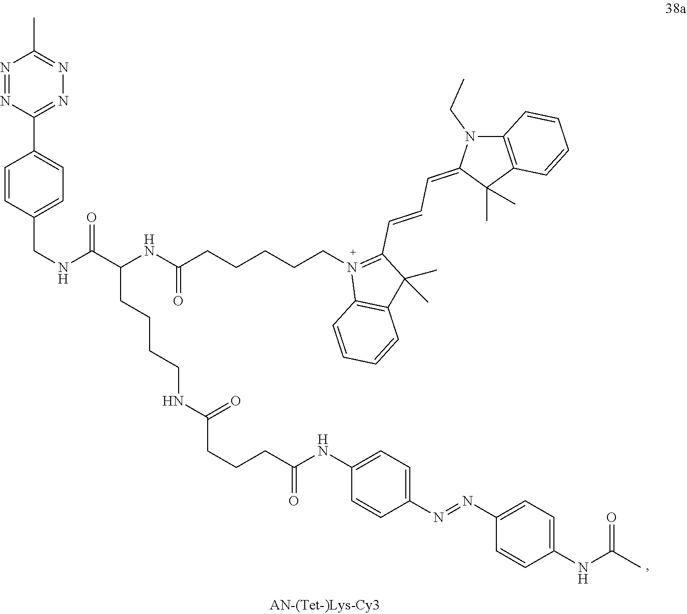

38a was synthesized according to GP-C4, using:
   as fluorophore carboxylic acid: Cy3 carboxylic acid (2.77 mg, 5.88 μmol, 1.05 eq., 0.029 M in DMF)
   as coupling reagent: HBTU (2.12 mg, 5.60 μmol, 1.00 eq., 12.6 mg/mL in DMF)
   as C3: 34 (AN-(Tet-)Lys-NH₂, 4.00 mg, 5.04 μmol, 0.90 eq., in 100 μL DMF) incubation time after the addition of C3: 2 h

38a (AN-(Tet-)Lys-Cy3, 1.01 mg, 0.89 μmol, 16%) was obtained as a red/pink solid.

HRMS (ESI): m/z C₆₆H₇₈N₁₃O₅⁺[M]⁺: calc.: 1132.62434, found: 1132.62274.

### 1-(6-((6-(5-((4-((4-acetamidophenyl)diazenyl)phenyl)amino)-5-oxopentanamido)-1-((4-(6-methyl-1,2,4,5-tetrazin-3-yl)benzyl)amino)-1-oxohexan-2-yl)amino)-6-oxohexyl)-2-((1 E,3E)-5-((E)-1-ethyl-3,3-dimethylindolin-2-ylidene)penta-1,3-dien-1-yl)-3,3-dimethyl-3H-indol-1-ium (39a, AN-(Tet-)Lys-Cy5)

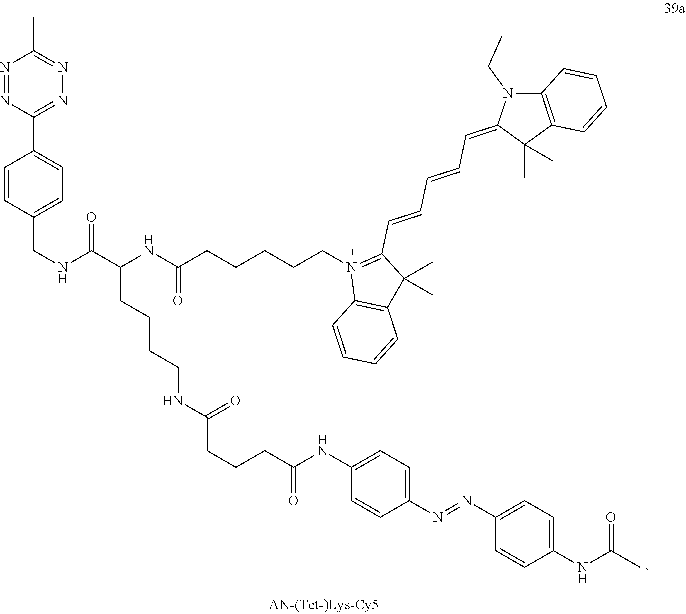

39a was synthesized according to GP-C4, using:
as fluorophore carboxylic acid: Cy5 carboxylic acid (2.93 mg, 5.88 μmol, 1.05 eq., 0.029 M in DMF)
as coupling reagent: HBTU (2.12 mg, 5.60 μmol, 1.00 eq., 12.6 mg/mL in DMF)
as C3: 34 (AN-(Tet-)Lys-NH₂, 4.00 mg, 5.04 μmol, 0.90 eq., in 100 μL DMF) incubation time after the addition of C3: 5 h

39a (AN-(Tet-)Lys-Cy5, 1.55 mg, 1.34 μmol, 27%) was obtained as a blue/green solid.

HRMS (ESI): m/z C₆₈H₈₀N₁₃O₅⁺ [M]⁺: calc.: 1158.63999, found: 1158.63814.

### 1-(6-((5-((tert-butoxycarbonyl)amino)-1-carboxypentyl)amino)-6-oxohexyl)-2-((1 E,3E)-5-((E)-1-ethyl-3,3-dimethylindolin-2-ylidene)penta-1,3-dien-1-yl)-3,3-dimethyl-3H-indol-1-ium (40)

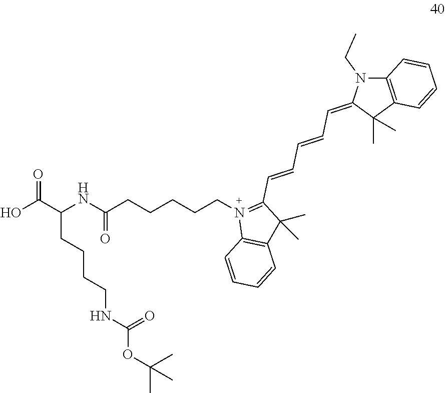

To a solution of Cy5 carboxylic acid (35.7 mg, 71.7 μmol, 1.1 eq.) in DMF (1.0 mL, 0.07 M) was added at room temperature, TSTU (21.6 mg, 71.7 μmol, 1.0 eq.) and DIPEA (51.5 mL, 287.0 μmol, 4.0 eq.). The reaction mixture was vortexed and incubated for 1 h, before a solution of commercial N-(tert-butoxycarbonyl)-L-lysine (20.0 mg, 78.9 μmol, 1.1 eq.) in DMF (0.5 mL, 0.16 M) was added. The reaction mixture was stirred at room temperature overnight, concentrated under a gentle stream of nitrogen and purified by silica gel flash column chromatography (DCM/MeOH: 100/0 to 50/50) yielding 40 (43.8 mg, 60.3 μmol, 84%) as a blue solid.

HRMS (ESI): m/z C₄₄H₆₁N₄O₅⁺[M]⁺: calc.: 725.46365, found: 725.46272.

### 1-(6-((5-amino-1-carboxypentyl)amino)-6-oxohexyl)-2-((1 E,3E)-5-((E)-1-ethyl-3,3-dimethylindolin-2-ylidene)penta-1,3-dien-1-yl)-3,3-dimethyl-3H-indol-1-ium (41)

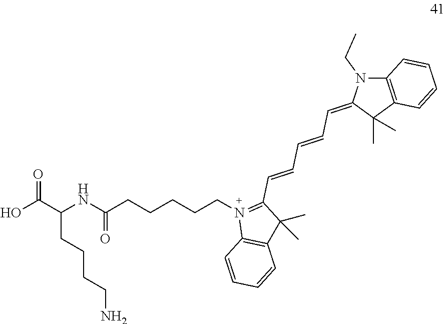

TFA (0.23 mL) was added dropwise to a solution of 40 (43.8 mg, 60.3 μmol, 1.0 eq.) in DCM (2.5 mL, 0.024 M) at room temperature. The reaction mixture was stirred until full conversion of the starting material was confirmed by LC/MS. The reaction mixture was then concentrated under reduced pressure, co-evaporated with toluene (2×1 mL) and filtered over silica (MeOH/AcOH: 100/0 to 99/1) to yield 41 (20.9 mg, 33.4 μmol, 55%) as a blue solid. The crude product was used for subsequent experiments without further purification.

HRMS (ESI): m/z C₃₉H₅₃N₄O₃⁺ [M]⁺: calc.: 625.41122, found: 625.41077.

### 1-(6-((5-(4-(4-((4-butylphenyl)diazenyl)phenyl)butanamido)-1-carboxypentyl)amino)-6-oxohexyl)-2-((1 E,3E)-5-((E)-1-ethyl-3,3-dimethylindolin-2-ylidene)penta-1,3-dien-1-yl)-3,3-dimethyl-3H-indol-1-ium (42)

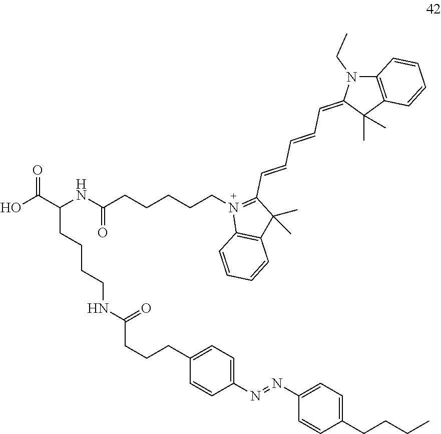

To a solution of 21 (AK, 3.89 mg, 12.0 μmol, 1.2 eq.) in DMF (0.15 mL, 0.08 M) were added at room temperature, a solution of TSTU (3.01 mg, 10.0 μmol, 1.0 eq.) in DMF (0.1 g/mL) and DIPEA (6.96 μL, 39.9 μmol, 4.0 eq.). The mixture was vortexed and incubated for 1 h, before a solution of 41 (5.0 mg, 8.0 μmol, 0.8 eq.) in DMF (0.25 mL, 0.03 M) was added. The reaction mixture was stirred over night, concentrated under a gentle stream of nitrogen and purified by silica gel flash column chromatography (DCM/MeOH: 90/10 to 70/30) yielding 42 (4.1 mg, 4.4 μmol, 55%) as a blue/green solid.

HRMS (ESI): m/z C₅₈H₇₅N₆O₄⁺[M]⁺: calc.: 931.58443, found: 931.58398.

### 1-(6-((6-(4-(4-((4-butylphenyl)diazenyl)phenyl)butanamido)-1-((4-(6-methyl-1,2,4,5-tetrazin-3-yl)benzyl)amino)-1-oxohexan-2-yl)amino)-6-oxohexyl)-2-((1 E,3E)-5-((E)-1-ethyl-3,3-dimethylindolin-2-ylidene)penta-1,3-dien-1-yl)-3,3-dimethyl-3H-indol-1-ium (43a, AK-(Tet-)Lys-Cy5)

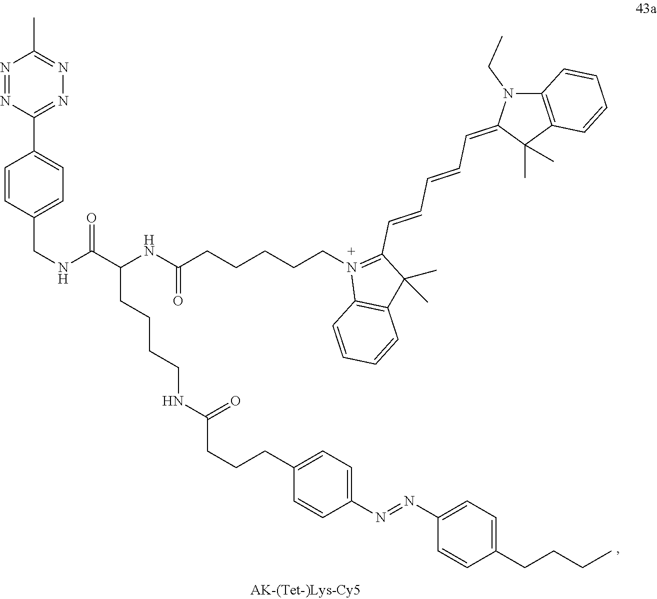

To a solution of 42 (4.10 mg, 4.40 μmol, 1.00 eq.) in DMF (0.7 mL, 0.006 M) were added at room temperature, a solution of HBTU (1.75 mg, 4.61 μmol, 1.05 eq.) in DMF (17.5 mg/mL) and DIPEA (4.59 μL, 26.4 μmol, 6.0 eq.). The mixture was vortexed and incubated for 1 h, before commercial methyltetrazine-amine hydrochloride (1.04 mg, 4.39 μmol, 1.00 eq.) was added. The reaction mixture was stirred at room temperature for 18 h, concentrated under a gentle stream of nitrogen and purified by silica gel flash column chromatography (DCM/MeOH: 100/0 to 90/10) yielding 43a (AK-(Tet-)Lys-Cy5, 1.24 mg, 1.11 μmol, 25%) as a blue/green solid.

HRMS (ESI): m/z C₆₉H₈₄N₁₁O₃⁺[M]⁺: calc.: 1114.67531, found: 1114.67369.

### 4.7 Synthesis of Azoaryl Tetrazines and Labelling of Photostabiliser Strands

### N-methyl-N-(4-((4-sulfophenyl)diazenyl)phenyl)glycine (44)

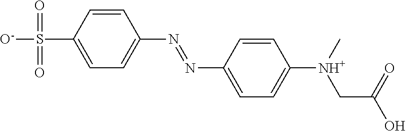 [SMILES: C[NH+](CC(=O)O)c1ccc(/N=N/c2ccc(S(=O)(=O)[O-])cc2)cc1]

Sulfanilic acid (520 mg, 3 mmol, 1.0 eq.) was added to aq. HCl (2 M, 3.6 mL) and methanol (2 mL) and cooled to 0° C. before adding aq. NaNO₂ (2 M, 1.58 mL, 1.05 eq.). The reaction mixture was stirred for 0.5 h and then dropwise added into a mixture of 2-(methyl(phenyl)amino) acetic acid (496 mg, 3 mmol, 1.0 eq.) and sodium acetate trihydrate (2.04 g, 15 mmol, 5 eq.) in water (10 mL) at 0° C. After 1 h, the mixture was concentrated under reduced pressure and the resulting crude product was purified by reverse phase flash column chromatography (H₂O/MeCN+0.1% FA, 5→100% MeCN) yielding 44 (122 mg, 0.349 mmol, 12%) as a red solid.

LCMS (ESI): tᵣₑₜ=4.36 min, 350 m/z [M+H]⁺.

### 4-((4-(methyl(2-((4-(6-methyl-1,2,4,5-tetrazin-3-yl)benzyl)amino)-2-oxoethyl)amino)phenyl)diazenyl)benzenesulfonic acid (45)

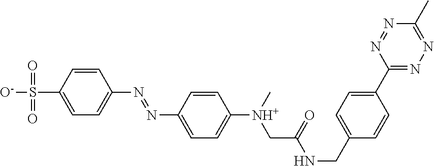 [SMILES: Cc1nnc(-c2ccc(CNC(=O)C[NH+](C)c3ccc(/N=N/c4ccc(S(=O)(=O)[O-])cc4)cc3)cc2)nn1]

44 (22.0 mg, 0.0631 mmol, 1.0 eq.), and HATU (26.4 mg, 0.0694 mmol, 1.1 eq.) were dissolved in DMF (2 mL) and DIPEA (0.044 mL, 0.25 mmol, 4.0 eq.) was added. After 5 minutes, (4-(6-methyl-1,2,4,5-tetrazin-3-yl)phenyl)methanamine hydrochloride (15.0 mg, 0.0631 mmol, 1.0 eq.) was added. The reaction mixture was stirred for 70 h, then the mixture was concentrated under reduced pressure and the resulting crude product was purified by reverse phase flash column chromatography (H₂O/MeCN+0.1% FA, 5→100% MeCN) yielding 45 (3.5 mg, 0.0066 mmol, 10%) as a red solid.

R_{f}=0.44 [H₂O:MeCN, 1:1 (+0.1% FA)]. LCMS (ESI): tᵣₑₜ=5.03 min, 531 m/z [M−H]⁻.

### 4-(4-((4-butylphenyl)diazenyl)phenyl)-N-(4-(6-methyl-1,2,4,5-tetrazin-3-yl)benzyl)butanamide (46)

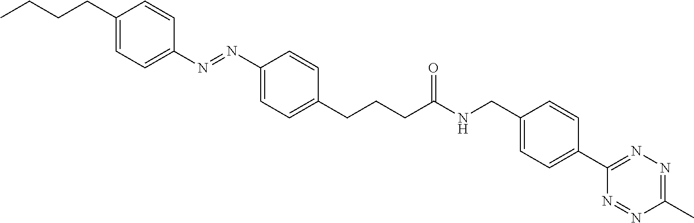 [SMILES: CCCCc1ccc(/N=N/c2ccc(CCCC(=O)NCc3ccc(-c4nnc(C)nn4)cc3)cc2)cc1]

FAAzo4(Frank; Nat Commun 2015 6, 7118) (20.5 mg, 0.0631 mmol, 1.0 eq.), and HATU (26.4 mg, 0.0694 mmol, 1.1 eq.) were dissolved in DMF (2 mL) and DIPEA (0.044 mL, 0.25 mmol, 4.0 eq.) was added. After 5 minutes, (4-(6-methyl-1,2,4,5-tetrazin-3-yl)phenyl)methanamine hydrochloride (15.0 mg, 0.0631 mmol, 1.0 eq.) was added. The reaction mixture was stirred for 70 h, diluted with ethyl acetate (20 mL), separated against a half-saturated aqueous sodium bicarbonate solution (40 mL; i.e. a 1:1 mixture of saturated aqueous sodium bicarbonate solution and water), and washed twice with 10% LiCl (2×20 mL) and brine (2×20 mL). The combined organic phases were dried over Na₂SO₄ and concentrated under reduced pressure. Purification of the resulting crude product by flash column chromatography (CH₂Cl₂/MeOH gradient, 0→20% MeOH) gave 46 (15 mg, 0.0295 mmol, 47%) as a red solid.

R_{f}=0.50 [CH₂Cl₂:MeOH, 19:1]. LCMS (ESI): tᵣₑₜ=8.27 min, 508 m/z [M+H]⁺.

### N-(4-(6-methyl-1,2,4,5-tetrazin-3-yl)benzyl)-4-(4-((4-propoxyphenyl)diazenyl)phenoxy)-butanamide (47)

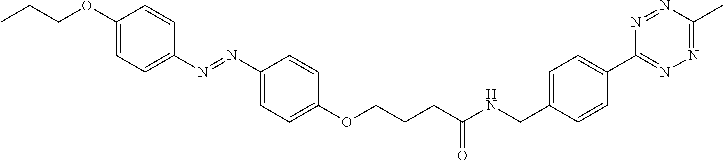 [SMILES: CCCOc1ccc(/N=N/c2ccc(OCCCC(=O)NCc3ccc(-c4nnc(C)nn4)cc3)cc2)cc1]

4-(4-((4-propoxyphenyl)diazenyl)phenoxy)butanoic acid (15.0 mg, 0.0438 mmol, 1.0 eq.) prepared following known procedures (Kunitake; J. Am. Chem. Soc. 1983 105, 6070-6078), and HATU (18.3 mg, 0.0482 mmol, 1.1 eq.) were dissolved in DMF (2 mL) and DIPEA (0.031 mL, 0.18 mmol, 4.0 eq.) was added. After 5 minutes, (4-(6-methyl-1,2,4,5-tetrazin-3-yl)phenyl)methanamine hydrochloride (10.4 mg, 0.0438 mmol, 1.0 eq.) was added. The reaction mixture was stirred for 70 h, diluted with ethyl acetate (20 mL), separated against half-saturated aqueous sodium bicarbonate solution (40 mL), and washed twice with 10% LiCl (2×20 mL) and brine (2×20 mL). The combined organic phases were dried over Na₂SO₄ and concentrated under reduced pressure. Purification of the resulting crude product by flash column chromatography (CH₂Cl₂/MeOH gradient, 0→20% MeOH) gave 47 (9.1 mg, 0.0173 mmol, 40%) as a red solid.

R_{f}=0.47 [CH₂Cl₂:MeOH, 19:1]. LCMS (ESI): tᵣₑₜ=7.70 min, 526 m/z [M+H]⁺.

### N-(4-(6-methyl-1,2,4,5-tetrazin-3-yl)benzyl)-11,12-dihydrodibenzo[c,g][1,2]diazocine-2-carboxamide (48)

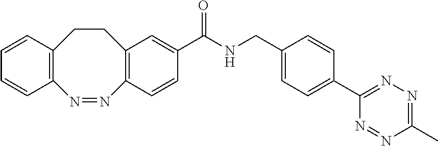 [SMILES: Cc1nnc(-c2ccc(CNC(=O)c3ccc4c(c3)CCc3ccccc3/N=N\4)cc2)nn1]

11,12-dihydrodibenzo[c,g][1,2]diazocine-2-carboxylic acid (15.9 mg, 0.0631 mmol, 1.0 eq.), and HATU (26.4 mg, 0.0694 mmol, 1.1 eq.) were dissolved in DMF (2 mL) and DIPEA (0.044 mL, 0.25 mmol, 4.0 eq.) was added. After 5 minutes, (4-(6-methyl-1,2,4,5-tetrazin-3-yl)phenyl)methanamine hydrochloride (15.0 mg, 0.0631 mmol, 1.0 eq.) was added. The reaction mixture was stirred for 70 h, diluted with ethyl acetate (20 mL), washed with half-saturated aqueous sodium bicarbonate solution (40 mL), and washed twice with 10% LiCl (2×20 mL) and brine (2×20 mL). The combined organic phases were dried over Na₂SO₄ and concentrated under reduced pressure. Purification of the resulting crude product by flash column chromatography (CH₂Cl₂/MeOH gradient, 0→20% MeOH) gave 48 (21 mg, 0.048 mmol, 76%) as a pink solid.

R_{f}=0.50 [CH₂Cl₂:MeOH, 19:1]. LCMS (ESI): tᵣₑₜ=6.68 min, 436 m/z [M+H]⁺.

### 2-(4-((4-(bis(2-hydroxyethyl)amino)phenyl)diazenyl)phenyl)-N-(4-(6-methyl-1,2,4,5-tetrazin-3-yl)benzyl)acetamide (49)

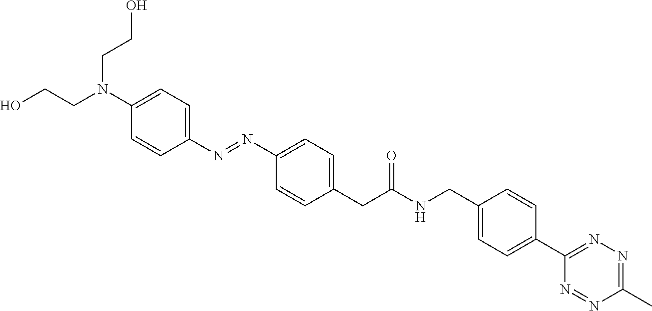 [SMILES: Cc1nnc(-c2ccc(CNC(=O)Cc3ccc(/N=N/c4ccc(N(CCO)CCO)cc4)cc3)cc2)nn1]

4-aminophenylacetic acid (227 mg, 1.5 mmol, 1.0 eq.) was added to aq. HCl (2 M, 1.8 mL) and methanol (2 mL) and cooled to 0° C. before adding aq. NaNO₂ (2 M, 0.79 mL, 1.05 eq.). The reaction mixture was stirred for 0.5 h and then dropwise added into a mixture of N-phenyldiethanolamine (272 mg, 1.5 mmol, 1.0 eq.), aq. NaOH (2 M, 1 mL) in water (5 mL) and methanol (5 mL) at 0° C. After 1 h, the mixture was diluted with ethyl acetate (60 mL), sat. aq. NH₄Cl (20 mL) and water 10 mL the organic phase was washed with brine (10 mL), dried over Na₂SO₄ and concentrated to give the crude azobenzene intermediate.

The intermediate (22 mg, 0.0642 mol, 1.02 eq.) and HATU (26.4 mg, 0.0694 mmol, 1.1 eq.) were dissolved in DMF (2 mL) and DIPEA (0.044 mL, 0.25 mmol, 4.0 eq.) was added. After 5 minutes, (4-(6-methyl-1,2,4,5-tetrazin-3-yl)phenyl)methanamine hydrochloride (15.0 mg, 0.0631 mmol, 1.0 eq.) was added. The reaction mixture was stirred for 18 h and the mixture was concentrated under reduced pressure and the resulting crude product was purified by flash column chromatography (CH₂Cl₂/MeOH gradient, 0→10% MeOH) yielding 49 (3.6 mg, 0.0068 mmol, 11%) as a red solid.

R_{f}=0.29 [CH₂Cl₂:MeOH, 9:1]. LCMS (ESI): tᵣₑₜ=6.20 min, 527 m/z [M+H]⁺.

The azoaryl-tetrazines 45-49 were then labelled onto 21-nt DNO oligos with 5′-TCO by the same procedure as described above to generate 21-nt oligos 45b-49b, such that when a 8-nt fluorophore strand (with 3′-amine fluorophore) and a 21-nt azoaryl strand (5′-azoaryl) are scaffolded together by the long complementary strand, their fluorophore and azoaryl motifs are reasonly near one another.

### 4.8 Solution-State Azoaryls

Alizarin Yellow (Alizarin Y) was purchased from BLDpharm (Product BD112961-25g, Lot BGZ779) and purified by reverse phase HPLC (H₂O/MeCN+0.1% formic acid, 15 min gradient 10→100% MeCN) yielding the pure compound as a red solid.

Orange G was purchased from Sigma Aldrich (Product 03756-25G, Lot MKBZ66364V) and recrystallized from ethanol containing 3% H₂O. The mixture was left at 4° C. overnight to yield red plate-shaped crystals, which were collected by filtration and washed with cold ethanol.

Alternatively, Orange G was also purified by reverse phase HPLC (H₂O/MeCN+0.1% formic acid, 15 min gradient 10→100% MeCN) yielding the pure compound.

Methyl Orange was purchased from AppliChem GmbH (Product 131431.1606, Lot 0000969249) and recrystallized from ethanol containing 3% H₂O. The mixture was left at 4° C. overnight to yield orange plate-shaped crystals, which were collected by filtration and washed with cold ethanol. Alternatively, it was purified by reverse phase HPLC (H₂O/MeCN+0.1% formic acid, 15 min gradient 10→100% MeCN) to give the pure azobenzene.

Acid Yellow 9 was purchased from abcr GmbH (Product AB576584, Lot 1467263) and directly used as purchased.

Water-soluble PST-1P and PST-2S were synthesised as reported (Borowiak; Cell 2015 162, 403-411).

Tartrazine was purchased from BLDpharm (Product BD01427245-25g, Lot CMP501) and purified by reverse phase high-performance liquid chromatography (H₂O/MeCN+0.1% formic acid, 15 min gradient 10→100% MeCN) yielding the pure tartrazine as orange solid.

### CS196

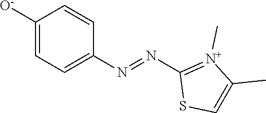 [SMILES: Cc1csc(/N=N/c2ccc([O-])cc2)[n+]1C]

An azo coupling was adapted from a previously described procedure (Alshargabi; Molecular Crystals and Liquid Crystals 2013 575, 128-139): 2-amino-4-methylthiazole (114 mg, 1.0 mmol, 1.0 eq.) was dissolved in AcOH (3 mL) and aqueous H₂SO₄ (60%, 4 mL) was added, and the solution was cooled down to 0° C. After dropwise addition of NaNO₂ (69 g, 1.0 mmol, 1.0 eq) in water (2.5 mL) the mixture was stirred for 1 h and then added dropwise to a cooled solution of phenol (0.38 g, 4.0 mmol, 1.0 eq.) in ethanol (3 mL). After 30 min of stirring at 0° C. the pH was adjusted to 5-6 by dropwise addition of aqueous NaOH (1 M). The precipitate was filtered off and washed with water to yield 4-((4-methylthiazol-2-yl)diazenyl)phenol (66 mg, 0.30 mmol, 30%, LCMS (ESI): tᵣₑₜ=6.69 min, 220 m/z [M+H]⁺) as an orange solid.

4-((4-methylthiazol-2-yl)diazenyl)phenol (165 mg, 0.75 mmol, 1.0 eq.) was dissolved in acetone (10 mL) and K₂CO₃ (156 mg, 1.13 mmol, 1.5 eq.) was added. After stirring for 5 min, Me₂SO₄ (0.071 mL, 0.75 mmol, 1.0 eq.) was added and the mixture was stirred at 50° C. for 5 h and then cooled to room temperature. The reaction was quenched with saturated NaHCO₃ solution, extracted with EtOAc, dried over Na₂SO₄ and purified via column chromatography (hexanes:ethyl acetate gradient, 25→100% ethyl acetate) yielding CS196 (20 mg, 0.088 mmol, 12%, LCMS (ESI): tᵣₑₜ=4.66 min, 234 m/z [M+H]⁺) as an orange solid.

### 5 Fluorescence Correlation Spectroscopy (FCS)

### 5.1 FCS in Solution, not-Deoxygenated

Fluorescence correlation analysis was performed on a custom-built inverted confocal microscope based on Olympus IX-71 body (Olympus Deutschland GmbH). Samples were excited with 532 nm pulsed laser (LDH-P-FA-530B, PicoQuant GmbH) with a repetition rate of 80 MHz. After passing a single-mode fiber (P3-488PM-FC, Thorlabs GmbH), the laser light was circularly polarized by a linear polarizer (LPVISE100-A, Thorlabs GmbH) and a quarter-wave plate (AQWP05M-600, Thorlabs GmbH). The excitation power used for each measurement was adjusted and measured at the entrance of the microscope body by means of a neutral-density filter (ND06A, Thorlabs GmbH). The light was focused onto the sample by water immersion objective (UPlanSApo, 60XWO, NA 1.20, Olympus Deutschland GmbH). The emission was separated from the excitation beam by a dichroic beam splitter (zt532/640rpc, Chroma Technologies) and focused onto a 50 μm diameter pinhole (Thorlabs GmbH). The emission light was separated from scattered excitation light by a long pass filter (RazorEdge LP 532, Semrock) and split into two detection channels by a nonpolarizing 50:50 beam splitter (CCM1-BS013/M, Thorlabs GmbH). In each detection channel, the afterglow luminescence of the avalanche photodiode was blocked by a 750 nm short-pass filter (FES0750, Thorlabs GmbH). Emission was focused onto avalanche photodiodes (SPCM-AQRH-14-TR, Excelitas Technologies GmbH& Co. KG), and the signals were registered by a multichannel picosecond event timer (HydraHarp 400, PicoQuant GmbH). SymphoTime 64 (PicoQuant GmbH) was used as control and analysis software. The fluorescence correlation measurements were performed in Nunc Lab-Tek II Chambered Slides (Thermo Fisher) which were cleaned with 1 M KOH for 1 h, washed with 1×phosphate buffered saline (PBS) buffer four times, and passivated with 1 mg/mL BSA-biotin (Thermo Fisher). Measurements were performed in 50/50 water/acetonitrile solution at ˜200 pM concentrations of EY-AK or EY-P, under air and without deoxygenation.

The cross-correlation function (G(T)) of the signals recorded on two avalanche photodiodes was calculated with SymphoTime 64. The software computes the cross-correlation of each of the two channels with the other channel, respectively, using the formula

$$G ( τ ) = \frac{〈 I ( t ) I ( t + τ ) 〉}{{〈 I ( t ) 〉}^{2}} - 1$$

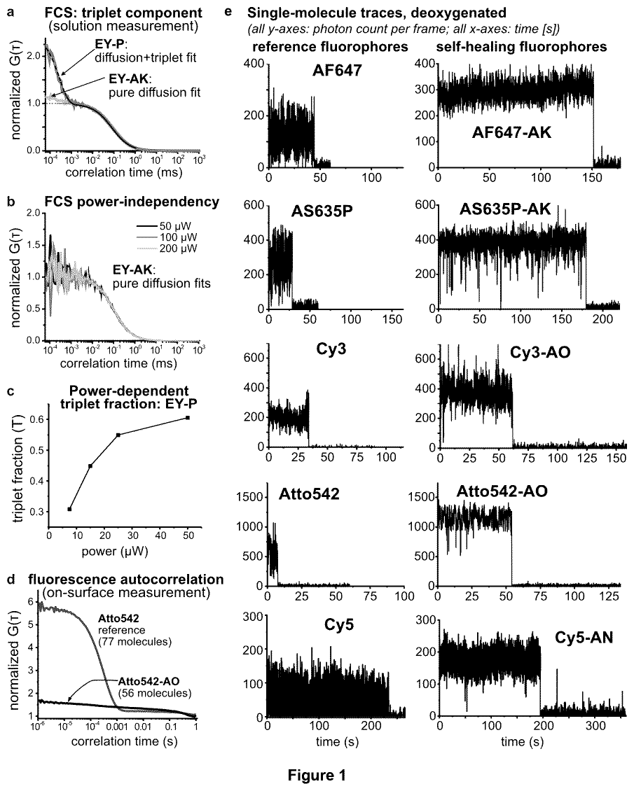

and then averages the result of both calculations to yield an averaged correlation function. These were normalized to the diffusion-only component (correlation time range 0.01-1 ms) and are plotted in FIG. 1a-b.

FIG. 1a shows that the FCS curve for stabilised molecule of the invention EY-AK fits a pure diffusion model that assumes no long-lived triplet states or other long-lived dark states are present (where long-lived implies lifetime 0.1 μs), returning a fitted diffusion time constant of 95 μs. The FCS curve for unstabilised EY-P only fits to a triplet+diffusion model which describes diffusion of fluorescent species with a significant component of a dark triplet state, returning fitted time constants of 81 μs for diffusion and 0.27 μs for the triplet (at 50 μW).

It is known that eosin Y (EY) has a high intersystem crossing yield (ca. 30%) to enter a long-lived dark triplet state, which can only relax spontaneously very slowly (lifetimes are typically quoted in the μs range) unless they are depleted either by e.g. collision with molecular oxygen, though that generates damaging reactive singlet oxygen as a byproduct, or else by photoexcitation to a typically damaging reactive higher excited state which is particularly likely under higher intensity imaging. The EY-P data indicate that relaxation of its triplet by oxygen and by photodepletion are very significant (observed lifetime 0.27 μs at 50 μW, or 1.2 μs at 7.5 μW [less photodepletion of the triplet at this lower excitation intensity]), matching the literature that states that high amounts of singlet oxygen are generated during illumination of eosin and that eosin is a photounstable and phototoxic species especially under high illumination intensity. By contrast, the absence of a detectable long-lived dark state FCS component for EY-AK indicates that any triplet states arising during excitation of the EY motif are very efficiently quenched by the azoaryl AK motif, much more efficiently than by molecular oxygen; therefore illumination of EY-AK should not generate as much singlet oxygen, and EY-AK is likely to have much higher photostability and instant brightness, compared to unstabilised EY.

FIG. 1b highlights that stabilised molecule of the invention EY-AK functions even under increasingly high photon intensity illumination as a good fluorophore, without any noticeable sign of long-lived triplet states (note that this suggests that it will cause low photogeneration of singlet oxygen under oxygenated conditions, and that it can have good photostability and high instant brightness and high photon budget).

FIG. 1c serves as a comparison to FIG. 1b, by showing that unstabilised EY-P has a very high triplet fraction even at a very low excitation power (7.5 μW) and that this fraction increases further as excitation intensity increases, whereas EY-AK had no detectable triplet fraction even at the vastly higher power of 200 μW (FIG. 1b). This highlights the need for and utility of the stabilisation brought about by the invention; and shows that the compound of the invention can perform as a self-healing dye even in the presence of molecular oxygen, which has been an unsolved challenge in self-healing dye design.

Therefore the invention can transform even “bad” fluorophores (EY) into an effective self-healing fluorescent dye where triplet states are no longer in evidence and better fluorescence performance is to be expected; furthermore, the compounds of the invention are effective even when excitation intensity increases (whereas the performance of typical dyes worsens as intensity increases).

### 5.2 Single Molecule Fluorescence Autocorrelation, Surface Immobilized, Deoxygenated

A sample chamber was created by attaching a cleaned glass slide via sticky tape onto a microscopy slide, such that a ca. 0.3 cm wide passage was formed. The surface of this chamber was functionalized with BSA-biotin (50 μL, 0.5 mg/mL in PBS, Sigma Aldrich, USA) and Neutravidin (50 μL, 0.25 mg/mL in PBS, Sigma Aldrich, USA). After hybridizing a 21-nt DNA oligo sequence of interest (labelled either at the 3′ or 5′ end with a fluorophore-azoaryl molecule of the invention, or else with a parent fluorophore [reference]) to a complementary biotin-labelled DNA strand (overnight incubation at 25° C. in PBS, 1:1 DNA molar ratio, total DNA strand concentration 200 nM), followed by dilution with 1× PBS (resulting DNA strand concentration 100 μM), the mixture containing the desired ds-DNA (labelled with both fluorophore and biotin) was attached to the prepared surface via biotin-neutravidin binding (surface treated with ds-DNA solution for 20 seconds, then washed with 200 μL of 2× PBS containing 500 mM NaCl and 0.05% Tween20). Oxygen was then removed using the GODCAT enzymatic oxygen scavenging system (glucose oxidase-catalase: 1% (wt/v) D-(+)-glucose (Sigma Aldrich, USA), 165 units/mL glucose oxidase (G2133, Sigma Aldrich, USA), 2170 units/mL catalase (C3155, Sigma Aldrich, USA), in 2× PBS buffer with 10 mM MgCl₂).

Single-molecule fluorescence measurements (autocorrelation of different blinking behaviors) were performed on a custom-built confocal microscope, based on an inverted microscope (IX-83, Olympus Corporation, Japan) and a 78 MHz-pulsed supercontinuum white light laser (SuperK Extreme, NKT Photonics A/S, Denmark) with selected wavelength of 532 nm. The wavelengths are selected via an acousto-optic tunable filter (AOTF, SuperK Dual AOTF, NKT Photonics A/S, Denmark). This is controlled by a digital controller (AODS 20160 8R, Crystal Technology, USA) via a computer software (AODS 20160 Control Panel, Crystal Technology, Inc., USA). A second AOTF (AA.AOTF.ns: TN, AA Opto-Electronic, France) was used to alternate 532 nm (for green channel) and 639 nm (for red channel) wavelengths, as well as to further spectrally clean the laser beam. It is controlled via self-written LabVIEW software (National Instruments, USA). A neutral density filter was used to regulate the laser intensity, followed by a linear polarizer and a λ/4 plate to achieve circularly polarized excitation. A dichroic beam splitter (ZT532/640rpc, Chroma Technology, USA) and an oil immersion objective (UPlanSApo 100×, NA=1.4, WD=0.12 mm, Olympus Corporation, Japan) were used to focus the excitation laser onto the sample. Nanopositioning was performed using a Piezo-Stage (P-517.3CL, E-501.00, Physik Instrumente GmbH&Co. KG, Germany). The excitation power of the 532 nm laser was set to 2 μW. Emitted light was collected using the same objective and filtered from the excitation light by the dichroic beam splitter. The light was later focused on a 50 μm pinhole (Linos AG, Germany) and detected using SinglePhoton Avalanche Diodes (SPCM, AQR 14, PerkinElmer, Inc., USA) registered by a TCSPC system (HydraHarp 400, PicoQuant GmbH, Germany) after additional spectral filtering (RazorEdge 647, Semrock Inc., USA for the red channel and BrightLine HC 582/75, Semrock Inc., USA for the green channel). A custom-made LabVIEW software (National Instruments, USA) was used to acquire the raw data. Confocal scans of 10×10 μm using a resolution of 2 ms/pixel and size of 50 nm/pixel were acquired. After single molecules were manually selected, temporal intensity trajectories were acquired using the Pick&Destroy tool of the setup software. The obtained data was further analysed using a self-written Python script allowing to determine the start and bleaching point of every single-molecule trajectory picked, which then calculates the autocorrelation curve for each trace. More than 50 autocorrelation curves were averaged to generate FCS autocorrelation graphs as shown in FIG. 1d.

FIG. 1d shows FCS autocorrelation data for the representative stabilised molecule of the invention Atto542-AO (that had been labelled to its object target 21-nt DNA strand by DBCO-azide click chemistry as detailed above, to generate strand Atto542-AO-21 nt) as compared to unstabilised Atto542 (from comparator Atto542-21 nt strand). Similarly to the solution-based FCS measurements in section 5.1, it will be seen that the dark states (triplet states or triplet-born dark states) associated to the fluorophore (trace Atto542, high G(T) at times T below 300 μs) are greatly suppressed or eliminated when the fluorophore is derivatised to become a compound of the invention (trace Atto542-AO, G(T) remains low even down to 1 μs). This is coherent with the 8-fold higher photon budget and 5-fold higher instantaneous brightness of Atto542-AO as compared to Atto542 which were recorded in the single-molecule localisation (cf. Table below); and it also indicates that under non-deoxygenated conditions, illumination of Atto542-AO should not generate as much singlet oxygen as Atto542 will.

### 6 Single-Molecule Imaging and Characterization of Photophysical Properties

### 6.1 Single Molecule Imaging with Self-Healing 21-Nt DNA Oligos, Deoxygenated

To enable single-molecule studies and detailed characterization of photophysical properties, the 21-nt DNA strand with fluorophore-azoaryl conjugates or parent fluorophores conjugated at the 3′-end or 5′-end were hybridized to a biotin-labelled complementary strand. These ds-DNA conjugates were immobilized on BSA-biotin coated glass coverslips via biotin-Neutravidin interaction as detailed in section 5.2. Oxygen was removed using GODCAT oxygen scavenging system (see 5.2). The strands were then imaged on a commercial Nanoimager S (ONI Ltd., UK), exciting the single molecules with 532 nm laser (for ATT0542 or Cy3 based molecules) or 639 nm laser (for AlexaFluor647, Cy5, AbberiorStar635P based molecules) using 9 mW laser power measured at the objective. The movies were recorded at 50 ms/frame time resolution, until photobleaching, as previously described (Scheckenbach; Angewandte Chemie International Edition 2021 60, 4931-4938). The data were analysed using ImageJ 1.53 to subtract the background and extract single-molecule fluorescence vs. time trajectories. The picked trajectories were analyzed with a custom Python code utilizing a Hidden Markov Model to identify the blinking and bleaching events. The photon budget until photobleaching, average count rate (brightness) and survival time were extracted for every single molecule. The ensemble histograms of all analyzed single molecules were obtained for these parameters by compiling data, with exponential decay fits to extract the average values for photon budget, or normal distribution fit to find mean values for instant brightness; within a fluorophore class (parent plus its fluorophore-azoaryl conjugates). In the following table, the average total photon count (TPC) and instant brightness (iBR) (from averaging across >100 analyzed single molecule trajectories per compound) are tabulated, for sets of parent and azoaryl-conjugated fluorophores; the compound number (cpd*) refers to the numbering of the labelled DNA strand being imaged (e.g. Ref8 for a parent, 38b for a self-healing dye, etc). The values reported for parent fluorophores are absolute emitted TPC and iBR; the values reported for all self-healing dyes are the fold values relative to their parent Ref, e.g. TPC=2.0 indicates twice as high photon budget as the unstabilised reference fluorophore.

| no O2 | Cy3- | | | Cy5- | | |
|---|---|---|---|---|---|---|
| azoaryl | cpd* | TPC | iBR | cpd* | TPC | iBR |
| none | Ref8 | 102000 ± | 195 ± | Ref3 | 56900 ± | 100 ± |
| (raw | | 1200 | 6 | | 3100 | 1 |
| counts) | | | | | | |
| -AK | 35b | 2.4 | 1.4 | 43b | 7.5 | 2 |
| -AN | 38b | 1.8 | 0.8 | 39b | 18.1 | 1.4 |
| -AO | 36b | 3.6 | 1.2 | 37b | 16 | 1.7 |

| no O2 | Atto542- | | | AS635P- | | | AF647- | | |
|---|---|---|---|---|---|---|---|---|---|
| azoaryl | cpd* | TPC | iBR | cpd* | TPC | iBR | cpd* | TPC | iBR |
| none | Ref7 | 68200 ± | 195 ± | Ref1 | 45200 ± | 58 ± | Ref2 | 105600 ± | 117 ± |
| (raw | | 3500 | 8 | | 2200 | 2 | | 3300 | 2 |
| counts) | | | | | | | | | |
| -AK | 12b | 2.6 | 2.9 | 15b | 16.8 | 5.7 | 14b | 6.7 | 3 |
| -AO | 17b | 7.9 | 4.8 | 20b | 16.3 | 5.6 | 19b | 6.5 | 1.7 |

The tabulated results notably show that the azoaryl-fluorophore conjugates of the invention can have up to >15-fold higher photon budget and >5-fold higher instant brightness than the reference fluorophores, across different fluorophore chemotypes and excitation colours. Especially considering that many of the reference fluorophores are widely in use as “best in class” fluorophores, these results show the value and practical utility of the invention.

Representative single molecule traces are shown in FIG. 1e for 5 self-healing molecules of the invention as well as their reference fluorophores under deoxygenated conditions [y-axis: photons collected per binning time]. These traces provide extra information about the utility of the invention that is not captured by averaging the photon budget (area under the curve before bleaching) or instantaneous brightness (average photon count before bleaching) over many molecules as was done to give the numerical values in the table. For example, the amplitude of the photon count trace (relative to its mean value) indicates the variability of fluorophore brightness, with higher variability expected when triplet states are longer-lived. When the emission trace amplitude is more variable, quantitative interpretation of imaging data will be more accurate and/or more rapid.

These traces illustrate that molecules of the invention show significantly enhanced instant brightness [higher average y-axis value] as well as signal stability, as well as typically ca. 2-to-10-fold higher photon budget (and ca. 2- to 5-fold longer survival time), and dye longevity [time before photobleaching] than the commercial fluorophores they are built around, which are already considered high-performance for single-molecule spectroscopy. Since the invention can outperform them in many respects, this indicates its high value.

### 6.2 Single Molecule Imaging with Self-Healing 21-Nt DNA Oligos, not Deoxygenated

Identical procedures as in the deoxygenated system (section 6.1) were used, except that no GODCAT oxygen scavenger was applied.

The tabulated results (formatting and abbreviations as defined above) notably show that the azoaryl-fluorophore conjugates of the invention even have substantially improved performance under oxygenated conditions (which current self-healing dye strategies struggle to achieve) compared to the parent fluorophores, across different fluorophore chemotypes and excitation colours. This again indicates the value and practical utility of the invention.

| with O2 | Cy3- | | | Cy5- | | |
|---|---|---|---|---|---|---|
| azoaryl | cpd* | TPC | iBR | cpd* | TPC | iBR |
| none | Ref8 | 17400 ± | 247 ± | Ref3 | 900 ± | 139 ± |
| (raw | | 130 | 3 | | 39 | 3 |
| counts) | | | | | | |
| -AK | 35b | 1.3 | 1.3 | 43b | 2.8 | 1.5 |
| -AN | 38b | 1.2 | 0.8 | 39b | 2.2 | 1 |
| -AO | 36b | 1.5 | 1.5 | 37b | 2.5 | 1 |

| with O2 | Atto542- | | | AS635P- | | | AF647- | | |
|---|---|---|---|---|---|---|---|---|---|
| azoaryl | cpd* | TPC | iBR | cpd* | TPC | iBR | cpd* | TPC | iBR |
| none | Ref7 | 180000 ± | 1592 ± | Ref1 | 80500 ± | 280 ± | Ref2 | 5620 ± | 170 ± |
| (raw | | 1700 | 24 | | 780 | 4 | | 230 | 5 |
| counts) | | | | | | | | | |
| -AK | 12b | 1 | 0.5 | 15b | 1.8 | 1.3 | 14b | 1.5 | 2 |
| -AO | 17b | 2 | 0.7 | 20b | 1.1 | 1.1 | 19b | 1.7 | 0.8 |

### 6.3 Single Molecule Imaging with Intermolecular Healing (Solution State Photostabilisation of Comparators)

Fluorophore-bearing reference 21nt DNA strands were immobilised and imaged as in the SMLM experiments in sections 6.1-6.2. Optionally, the following potential photostabilising solution-state additives were added, all at 2 mM: the commercial water-soluble azoaryls (listed with their CAS numbers) Alizarin Y (584-42-9), Orange G (1936-15-8), Methyl Orange (547-58-0), Acid Yellow 9 (2706-28-7), or Tartrazine (1934-21-0) (either high purity commercial product was used, or else commercial compound was additionally purified by preparative HPLC); or else azobenzenes PST-1P and PST-2S; or else the known non-azoaryl photostabilisation additives “TX/TQ” (Trolox/Trolox Quinone) or “COT”. The Trolox/Trolox quinone (TX/TQ) buffer was prepared by pre-dissolving 0.025 g of 6-hydroxy-2,5,7,8-tetramethylchroman-2-carboxylic acid in 1 mL of MeOH, mixing the solution with 50 mL of 2× PBS containing 10 mM MgCl₂, then illuminating it for 10-15 mins with UV light until the desired TQ concentration of ˜25 μM was achieved. The concentration was verified as described (Cordes; J. Am. Chem. Soc. 2009 131, 5018-5019). The “COT solution” was prepared as standard in the field by combining 1 μL of 200 mM COT (dissolved in DMSO) and 99 μL 2× PBS containing 10 mM MgCl₂.

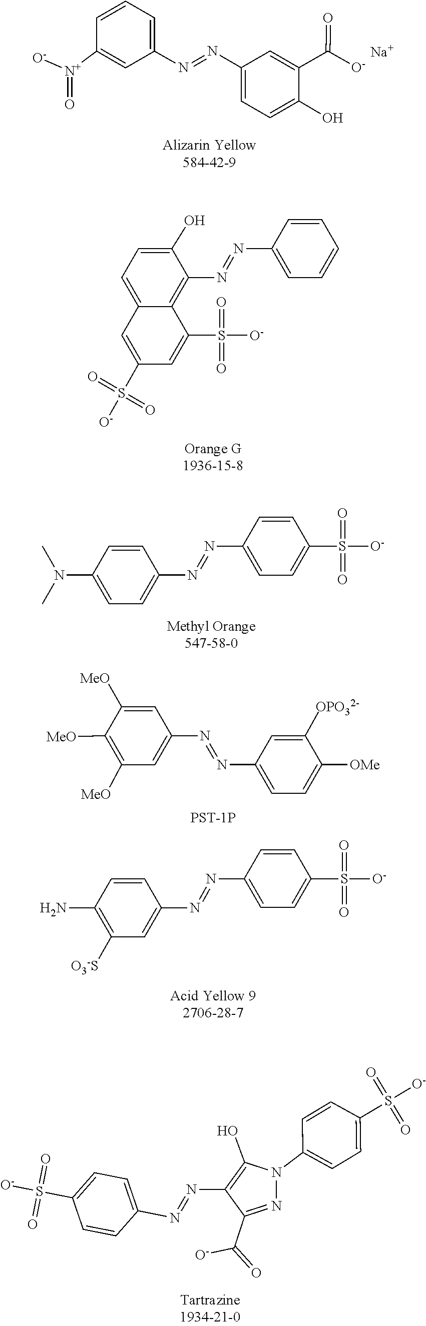

These intermolecular measurements are indicative of the performance that an intramolecular (self-healing) compound containing the same stabiliser and fluorophore would be capable of accessing, since here the relatively high concentration of the solution state stabiliser can ensure regular molecular collisions with the fluorophore, and thus report on the potential for photostabilisation. Therefore, these results are indicative of the utility that the self-healing design of the invention can reach, and are also useful for screening which combinations of fluorophore and azoaryl are likely to be most effective in a self-healing compound. The azoaryl additives were highly effective in increasing fluorophore performance.

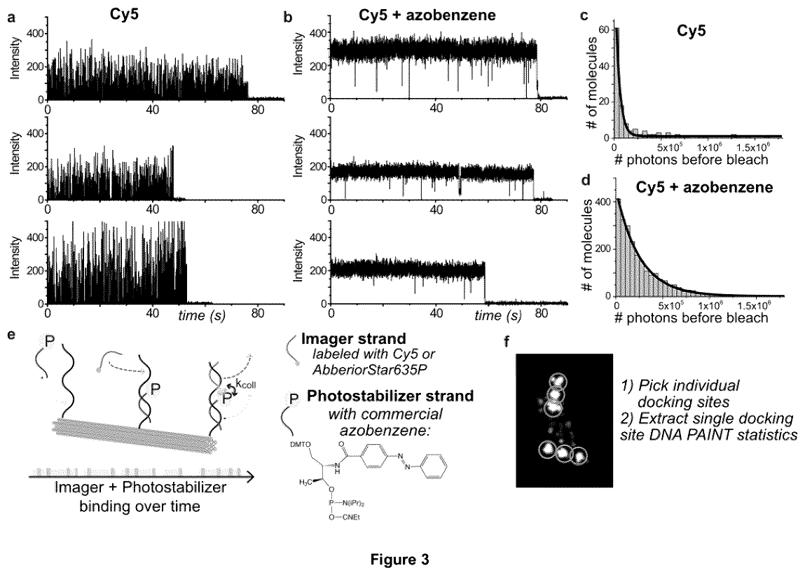

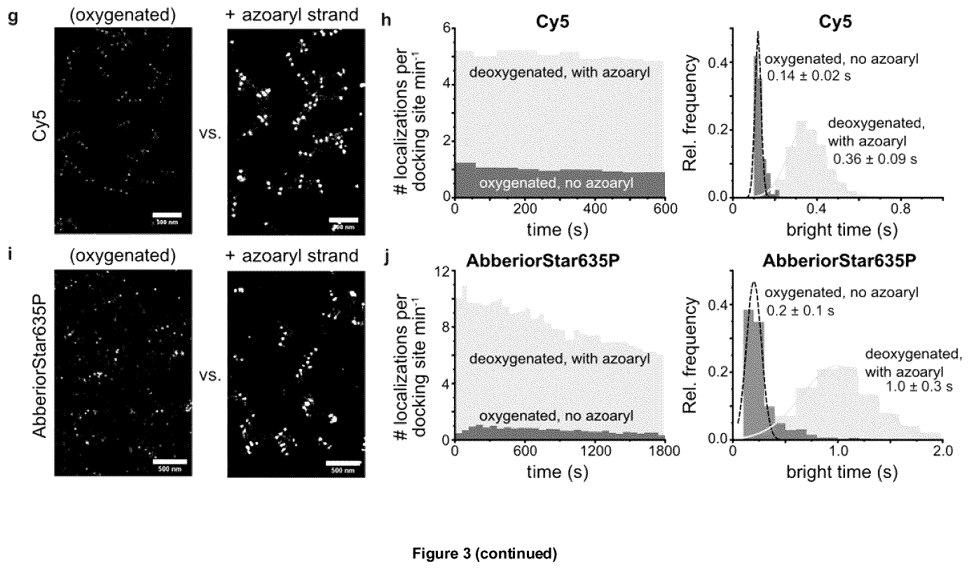

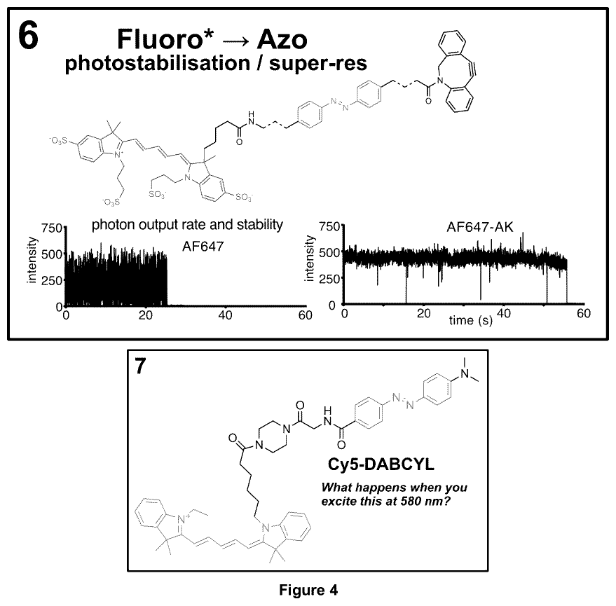

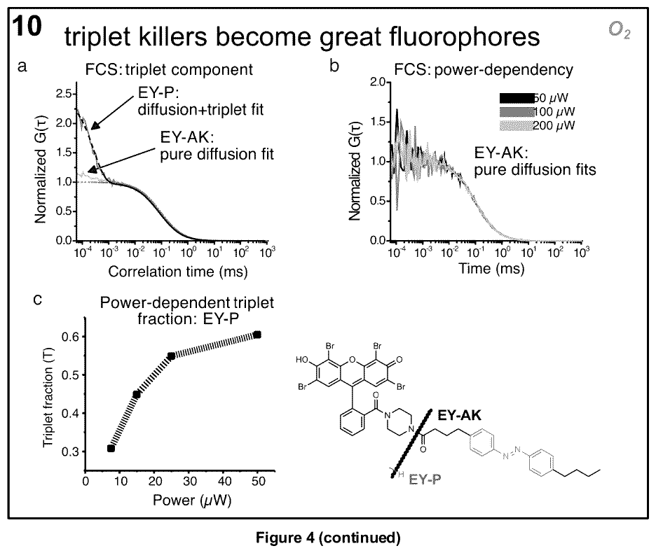

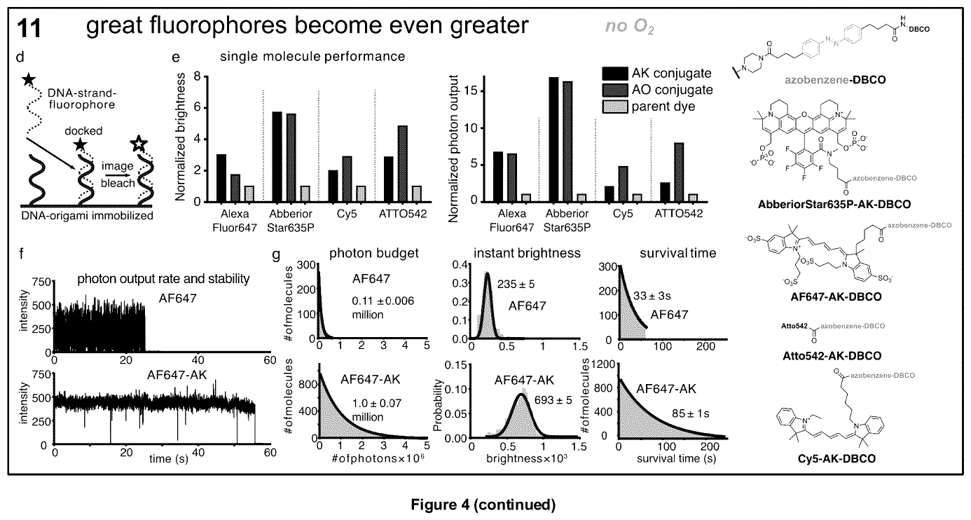

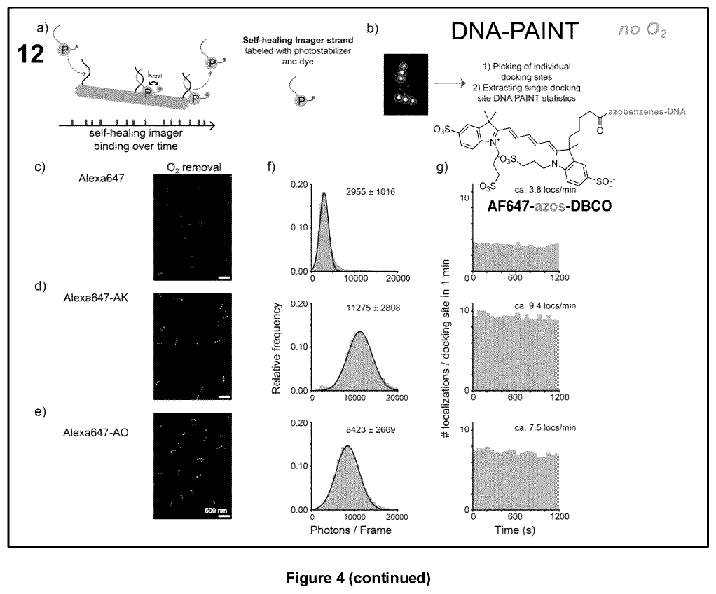

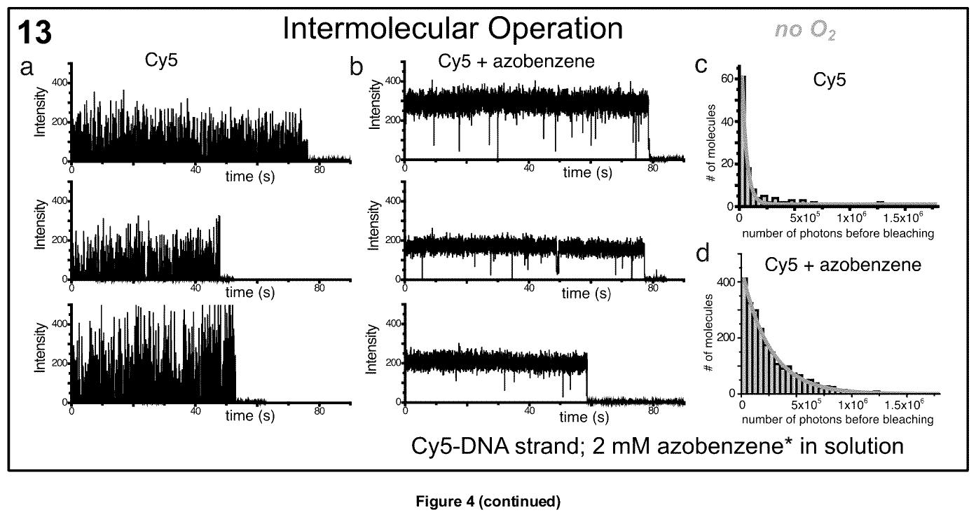

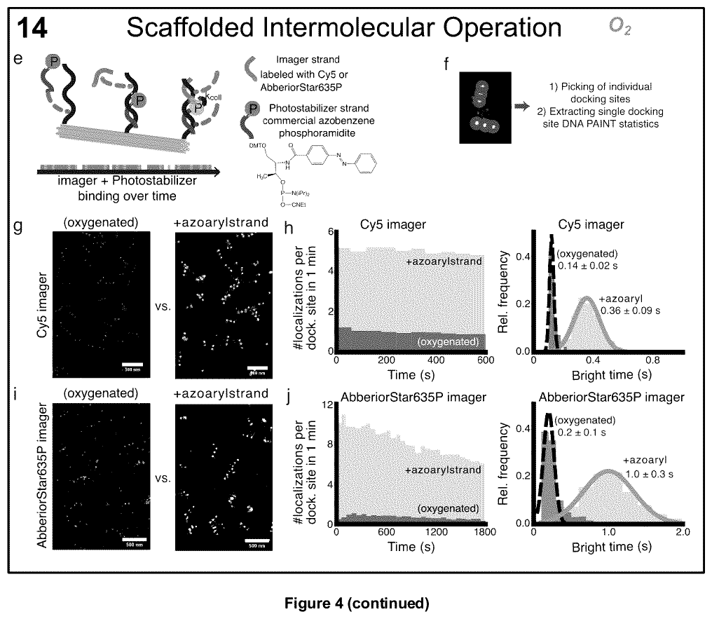

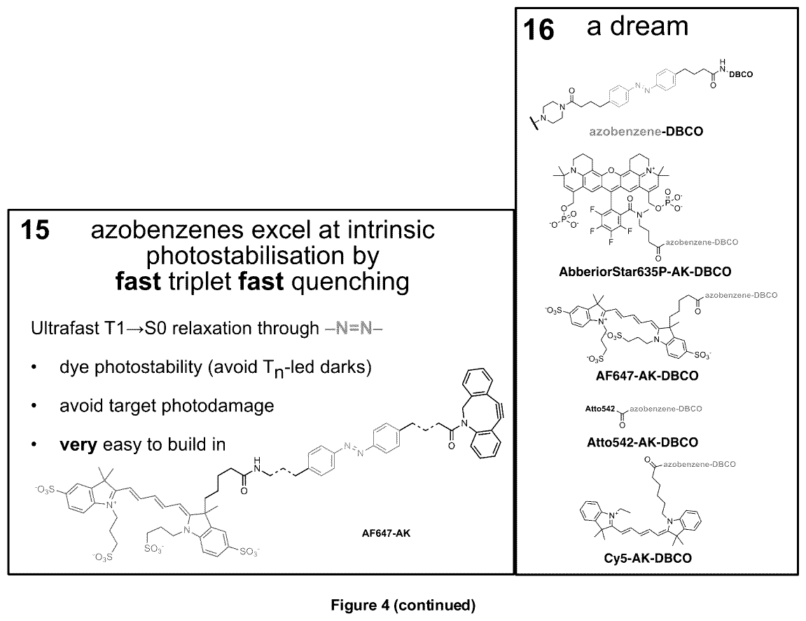

FIG. 3a-d shows example single-molecule trajectories and statistics for single molecule localisation experiments, imaging a Cy5-21 nt strand in GODCAT-deoxygenated samples optionally in the presence of azobenzene PST-2S (2 mM in solution) as a photoprotective agent. The signal intensity of Cy5 (FIG. 3a, photon counts per frame) is greatly increased as well as made more stable in the presence of the azobenzene (FIG. 3b); and the Cy5 photon budget distribution (FIG. 3c) is also shifted towards much larger photon budgets when the azobenzene is present (FIG. 3d). These features extend to most other fluorophore/azobenzene combinations tested under deoxygenated conditions: the photon budgets of even high-performance fluorophores are drastically increased, by ca. 5-fold (Atto488), 10-fold (AF647), or >15-fold (Cy3B); and so is instantaneous brightness:

### Solution-State Intermolecular Stabilisation, Deoxygenated

| | Atto488 | | Cy3B | | AF647 | |
|---|---|---|---|---|---|---|
| | TPC | iBR | TPC | iBR | TPC | iBR |
| Reference | 9830 ± | 726 ± | 47700 ± | 871 ± 6 | 224000 ± | 177 ± 2 |
| | 320 | 41 | 1700 | | 6000 | |
| TXTQ | 4.2 | 1.6 | 16.4 | 1.3 | 5.1 | 1.6 |
| COT | 0.7 | 1.0 | 16.7 | 1.6 | 5.3 | 1.5 |
| PST-1P | 4.7 | 1.6 | 16.9 | 1.4 | 11.6 | 2.0 |
| Alizarin Y | 5.7 | 1.8 | — | — | — | — |
| Orange G | — | — | 17.1 | 1.4 | 8.6 | 1.5 |
| Methyl Orange | — | — | 34.5 | 1.2 | 10.3 | 1.5 |
| Acid Yellow 9 | — | — | 14.9 | 1.2 | 7.5 | 1.4 |

Even under non-deoxygenated conditions, the photon budget particularly of Atto488 could be outstandingly enhanced by azoaryls (ca. 6- to 16-fold), and other performance aspects of all the fluorophores could be somewhat improved:

### Solution-State Intermolecular Stabilisation, not Deoxygenated

| | Atto488 | | Cy3B | | AF647 | |
|---|---|---|---|---|---|---|
| | TPC | iBR | TPC | iBR | TPC | iBR |
| Reference | 4232 ± | 1070 ± | 26520 ± | 1331 ± | 4760 ± | 320 ± 4 |
| | 50 | 50 | 570 | 16 | 120 | |
| TXTQ | 11.4 | 1.2 | 1.3 | 1.0 | 1.6 | 0.9 |
| COT | 2.0 | 1.4 | 1.0 | 0.8 | 0.8 | 0.9 |
| PST-1P | 10.6 | 1.2 | 0.9 | 0.7 | 1.3 | 0.9 |
| Alizarin Y | 10.5 | 1.1 | — | — | — | — |
| Orange G | 11.7 | 1.1 | 1.3 | 0.9 | 1.3 | 0.9 |
| Methyl | 14.4 | 1.3 | 1.7 | 0.9 | 1.5 | 0.9 |
| Orange | | | | | | |
| Acid Yellow 9 | 16.6 | 1.2 | 1.2 | 0.8 | 1.8 | 0.8 |
| Tartrazine | 5.9 | 1.5 | — | — | — | — |

These intermolecular experiments indicate that azoaryls can be highly effective as photostabilisers for a range of dye “colours” (Atto488: “blue” excitation channel; Cy3B: “green”; AF647: “red”), which taken together with the other data presented, indicates a unified mechanism by which azoaryls can act as efficient dye photostabilisers, and thus supports the efficacy and utility of the (intramolecular) self-healing constructs of the invention.

### 7 Superresolution DNA-PAINT Imaging with 8-Nt DNA Oligos, Intramolecular Self-Healing, Deoxygenated

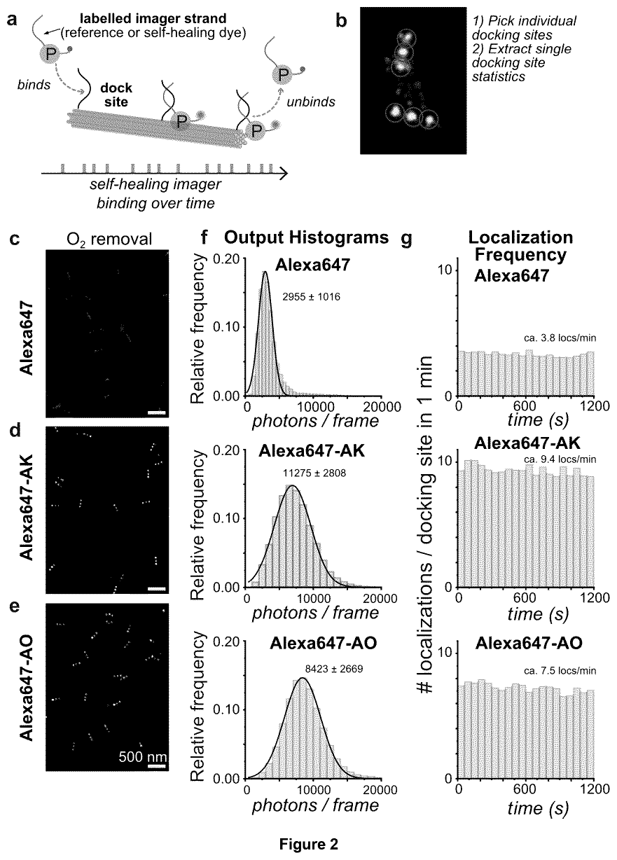

Photostability is of utmost importance in super-resolution imaging methods. Firstly, depopulation of the dark triplet states allows to improve the number of photons that can be extracted per fluorophore in localization-based super-resolution imaging thus leading to better resolution that can be achieved, and enhancing the chance for a given imager strand to pass the threshold for detection of localisation before it bleaches out and is replaced. Secondly, depopulation of the dark triplet states also allows improved chemical or biochemical stability of the docking site itself during imaging (due to minimized generation of reactive oxygen species by fluorophore triplet states), which permits each site to host more imager strand docking cycles before it is damaged and can no longer bind the imager. Taken together, a good self-healing dye should give higher resolution images and more localisations per minute. Exchangeable 8-nt DNA imager strands bearing parent fluorophores or self-healing compounds of the invention (e.g. 14c, 19c) were used in DNA-PAINT imaging (Jungmann; Nature Methods 2014 11, 313-318)), with GODCAT deoxygenation as above (FIG. 2a,b). DNA-PAINT measurements with standard 12-helix-bundle (12HB) DNA origami structures containing 3 docking sites (as shown in FIG. 2a) were carried out on a custom-built total internal reflection fluorescence (TIRF) microscope, based on an inverted microscope (IX71, Olympus) placed on an actively stabilized optical table (TS-300, JRS Scientific Instruments) and equipped with a nosepiece (IX2-NPS, Olympus) for drift suppression. The sample was excited at 644 nm with a 150 mW laser (iBeam smart, Toptica Photonics). The laser beam was spectrally cleaned up (Brightline HC 650/13, Semrock), directed over a dichroic mirror (zt 647 rdc, Chroma) and focused on the back focal plane of the objective (UPLXAPO 100×, NA=1.45, WD=0.13, Olympus). An additional ×1.6 optical magnification lens was applied to the detection path resulting in an effective pixel size of 101 nm. The fluorescence light was spectrally filtered with an emission filter (ET 700/75, Chroma). Image stacks in TIF format were recorded by an electron multiplying charge-coupled device camera (Ixon X3 DU-897, Andor), which was controlled with the software Micro-Manager 1.4. The measurements were done in PBS containing 500 mM NaCl at 5 nM concentration of 8-nt imager strands. The samples were excited with 0.8 kW/cm² laser intensities and 12 000 frames were acquired at the time resolution of 100 ms/frame (total imaging time of 20 minutes).

DNA-PAINT raw data were analyzed using the Picasso software package (Schnitzbauer; Nature Protocols 2017 12, 1198-1228). The raw TIFF file format movies were first analyzed with the “localize” software. For fitting the centroid position information of single point spread functions (PSF) of individual imager strands, the MLE (maximum likelihood estimation) analysis was used with a minimal net gradient of 5000 and a box size of 7 px for the 12HB measurements. The fitted localizations were further analyzed with the “render” software from Picasso. X-y-drift of the localizations was corrected with the RCC drift correction. Individual docking sites were picked and the corresponding pick region statistics were exported for further analysis. For photon distributions, localization in the first or last frame of individual binding events were filtered out since they exhibit lowered photon counts than full-on frames.

FIG. 2c-g show example results from AlexaFluor647 and its self-healing compounds of the invention. The compounds of the invention have substantially increased imaging quality (FIG. 2c-e), photon budget (FIG. 2f), and localisation frequency (FIG. 2g), compared to the parent fluorophore. Scale bars in FIG. 2c,d,e are 500 nm.

### 8 DNA-PAINT with 8-Nt DNA Oligos, DNA-Mediated Photostabilization, Deoxygenated

Exchangeable 8nt DNA imager strands labelled with Cy5 and AbberiorStar635P were used in DNA-PAINT imaging without exclusion of molecular oxygen. Optionally, a 21nt DNA strand bearing a simple azoaryl derivative as a photostabiliser (either e.g. 45b-49b or else a commercial azobenzamide as shown in FIG. 3e) was permanently scaffolded onto the surface-bound DNA strand, to bring the azoaryl into proximity with the dye of the exchangeable imager in the deoxygenated conditions (GODCAT as in section 5). DNA-PAINT measurements with Cy5 imager strand were carried out on the setup detailed in section 6 while exciting the samples with 1.8 kW/cm² 640 nm laser and acquiring 6000 frames at a time resolution of 100 ms/frame (total imaging time of 10 minutes). DNA-PAINT imaging with AberiorStar635P was acquired on the setup detailed in section 7 while exciting the samples with 1.8 kW/cm² 644 nm laser and acquiring 18 000 frames at a time resolution of 100 ms/frame (total imaging time of 30 minutes). Raw DNA-PAINT data were analyzed using Picasso software package as detailed in section 7, extracting the statistics from the docking sites on the underlying DNA nanostructure (FIG. 3f).

The image brightness is drastically enhanced with the azobenzamide strand (FIG. 3g,i) which also increases by many-fold the rate of detecting localisations, as well as the time that fluorophores spend in an emissive state during their binding event (bright time) (FIG. 3h,j).

This scaffolded intermolecular photostabilization is another supporting result for the invention (intramolecular), confirming the unified mechanism by which azoaryls act as photostabilisers, and highlighting the efficacy and utility expected for self-healing constructs of the invention.

### 9 Summary

The compounds of the invention are self-healing dyes that are constructed by attaching an azoaryl unit to a parent dye such that its optical properties are improved, particularly its optical properties that are most relevant in highly-demanding imaging, including instant brightness, signal stability, photon budget, and rate of detecting localisations. Therefore the compounds of the invention can be useful for high-performance imaging, e.g. delivering higher spatial and temporal resolution within a shorter experimental acquisition time, and with more confidence. It was shown that compounds of the invention improve these fluorescence performance parameters in oxygen-free conditions by even 10- to 25-fold compared to the parent fluorophore. It was also indicated that compounds of the invention can be effective in aerated conditions. It was even shown that dyes which are not good as fluorophores (EY) can have their drawbacks addressed by transformation to an azoaryl-stabilised self-healing construct of the invention (EY-AK). It is also expected that dyes for which the currently known photostabilisation strategies are not efficient, can nevertheless be photostabilised by derivatisation to compounds of the invention (experimentally indicated by the solution state screening results with Atto488). Therefore, the unifying advance of the invention, i.e. photostabilisation and fluorescence improvement by ensuring the proximity of an azoaryl unit with a fluorophore unit, provides a valuable general solution for problems in fluorescence imaging, especially high-end and high-demand single-molecule and superresolution imaging.

1. A compound comprising a fluorophore unit, an azoaryl unit, and optionally a tether unit, wherein the compound is a compound having a formulae (I-1) to (I-9):
wherein “Azo” denotes the azoaryl unit; “Attach” denotes the tether unit; “F” denotes the fluorophore unit; the straight or wavy lines denote a linker unit; an intersection between a straight and a wavy line marks a branch point in a linker unit; an asterisk on a unit indicates that the unit is bivalent;

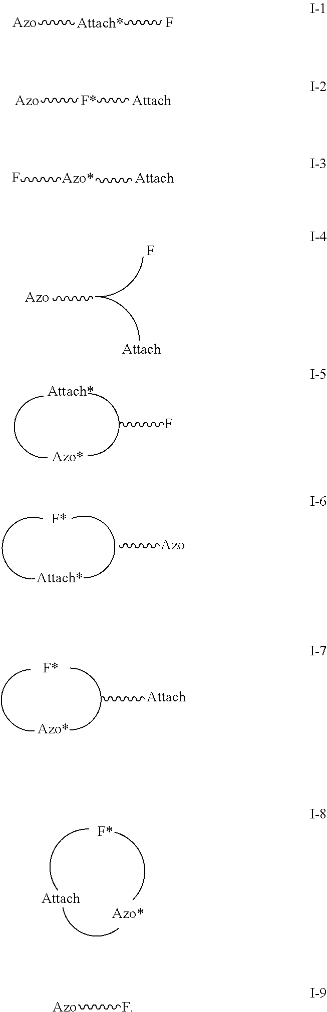

2. The compound according to claim 1, wherein the azoaryl unit is capable of depopulating a triplet state of the fluorophore.

3. The compound according to claim 1, wherein the azoaryl unit contains one, two, three, or four azoaryl species having the formula aryl-N═N-aryl′, wherein aryl and aryl′ are independently selected from an aryl group having 5 to 12 ring atoms selected from C, N, O and S; or
wherein the azoaryl unit contains one azoaryl species; or
wherein the azoaryl unit contains two, three, or four azoaryl species.

4. A compound according to claim 1, wherein the aryl rings of the azoaryl species are optionally substituted phenyl rings, wherein the substituents are independently selected from alkyl, halogen, ester, amide, nitrile, nitro, trifluoromethyl, alkoxy, amine, hydroxy, carboxylic acid, sulfonic acid, phosphonic acid, sulfonamide, carboxamide, and carboxylester.

5. A compound according to claim 1, wherein the azoaryl species are annelated.

6. A compound according to claim 1, wherein the azoaryl species are dibenzodiazepine, diazocine, or diazonine in which 0 to 2 of the carbon atoms in the bridge are replaced by heteroatoms chosen from N, O, Si, P, and S.

7. The compound according to claim 1, wherein the linker unit is independently selected from a covalent bond and a hydrocarbon linking moiety which comprises 1 to 48 atoms along the shortest linear path between the units that it connects, wherein the hydrocarbon linking moiety can optionally contain 1 to 16 heteroatoms selected from N, O and S and wherein the hydrocarbon linking moiety can be optionally substituted by one or more substituents,
wherein the linker units is preferably selected from —(CH₂)ₜ— (with t being 1 to 20), —O(CH₂)ₜO— (with t being 1 to 6), —(O)C(CH₂)ₜC(O)— (with t being 1 to 6), —(O)C(CH₂)ₜC(O)— (with t being 1 to 6), —(CH₂CH₂O)ₙ— (with n being 1 to 6), and poly(glycine) (—(C(O)CH₂NH)ₚ— with p being 1 to 6).

8. A compound according to claim 1, wherein the linker unit between the fluorophore unit and the azoaryl unit is a bond or an aliphatic group containing 1 to 8 non-hydrogen atoms, e.g. —O(CH₂)₁₋₂O—, —(CH₂)₁₋₄—, —(O)C(CH₂)₂C(O)—, —(CH₂CH₂O)₁₋₂—), preferably wherein the linker unit between the fluorophore unit and the azoaryl unit is an aliphatic group containing 1 to 4 non-hydrogen atoms.

9. The compound according to claim 1, wherein the fluorophore unit is selected from: cyanine (“Cy”) polymethines or their analogues including Cy3, Cy3.5, Cy3B, Cy5, AlexaFluor 555, AlexaFluor 647, Cy5.5, Cy7, Cy7.5, FNIR-tag, ICG, IRDye 8000W, SNIR1, IRDye 700, IRDye 78, DiO, Dil, DiO, DiR, MeOFlav7, and JuloFlav7; coumarin fluorophores including 4-methylumbelliferone, AlexaFluor 350, AlexaFluor430, pacific blue, Star 440 SXP, Atto 425, and Coumarin 153; BODIPY fluorophores including BODIPY FL, BODIPY 507/545, BODIPY TR, BDP R6G, BODIPY 558/568, BDP 581/591, BOPHY, C11-BODIPY, and BDP 650/665; xanthene-type fluorophores (including the fluorescein, rhodamine, rhodol, carbofluorescein, carborhodamine, carborhodol, silarhodamine, phosphorhodamine, pyronine, carbopyronine, and ketorhodamine subfamilies) including ATTO 647N, JF585, HMSiR, HM-DS655, SiP650, SiR 700, 680SiR, SiR680, HMSiR_{indol}, HMSiRⱼᵤₗₒₗ, HMSiR_{THQ}, Yale676_{sb}, Nebraska Red 700, HMSiR, HEtetTFER, Yale676sb, HMSiR_{THQ}, calcein, Fluo-4, fluorescein, oregon green, tokyo green, tetrachlorofluorescein, rhodol, Nebraska Red rhodol, Rhodamine 110, Rhodamine 6G, Rhodamine B, tetramethylrhodamine (TMR or TAMRA), JF526, AlexaFluor 488, AlexaFluor 546, AlexaFluor 594, DyLight488, JF549, Sulforhodamine 101, AlexaFluor 532, JFX549, Atto 647, Atto 488, CF 488, Rhodamine 800, Atto 550, HEtetTFER, Texas Red, HMJF526, HMRG, HMAcRG, JF593, sulfone-rhodamine, Atto 520, JF585, MaP555, Atto 610, CP550, SiP650, TMDHS, naphthofluorescein, AlexaFluor 568, Atto 594, and AlexaFluor 633; phenoxazine and benzophenoxazine fluorophores including Atto 655, Atto 680, resorufin, brilliant cresyl blue, Nile blue, Nile red, methylene blue, azure B, and azure A; metal-complexing fluorogenic fluorophores and their derivatives including Fura-1, Fura-2, calcein AM, Fura-2-acetoxymethyl ester, and Fluo-4 AM; and any derivatives of the the above that have identical π-system-chromophores but substitution patterns that tune physicochemical properties e.g. solubility or biolocalisation, or deuterated derivatives thereof.

10. The compound according to claim 1, wherein the tether unit is independently selected from a substrate for SNAPTag, CLIPTag, or HaloTag self-labelling proteins, or a bioorthogonally reactive moiety chosen from alkyne or strained alkyne, strained alkene, azide, phosphine, tetrazine, isothiocyanate, amine, NHS ester, benzylic fluoride, maleimide, acrylate, acrylamide, thiol, iodoacetamide, or chloroacetamide; lipid anchor (palmitoyl, cholesterol, prenyl), mitochondrial targeting delocalised lipophilic cation (rhodamine or triarylphosphonium), DNA-binding motif (oligo-benzimidazole or DAPI derivative), taxane, phalloidin, jasplankinolide, biotin, or desthiobiotin, or a DNA or PNA oligomer.

11. A compound according to claim 1, wherein the tether unit is a benzylguanine derivative for SNAP-tag labelling, a 6-chlorohexyl derivative for HaloTag-labelling, a benzylcytosine derivative for CLIP-tag labelling; or a strained alkene, alkyne, strained alkyne, azide, or tetrazine for click reactions; or an isothiocyanate, amine, NHS ester, benzylic halide, maleimide, acrylate, thiol, ethynylphosphonamidate, tetrafluorophenyl, pentafluorophenyl, 2-chloroacetamide, or 2-chloroacetate.

12. A compound according to claim 1, wherein the tether unit is biotin, desthiobiotin, a lipid motif containing 8 to 30 carbon atoms, a mitochondrial-targeting delocalised lipophilic cation including one based on a substituted triphenylphosphonium substituent, a DNA-binding motif, a taxane, a phalloidin, a jasplakinolide, or a short nucleic acid strand of 6 to 30 bases including a DNA strand.

13. A compound according to claim 1, wherein the tether unit is a substrate for a self-labelling protein tag (such as a HaloTag, CLIP-tag, or SNAP-tag) or is a bioorthogonally or biologically reactive moiety that is also useful for covalently labelling biomolecules; or is a moiety that is useful for noncovalently targeting specific cellular regions or environments.

14. A compound according to claim 1, where the tether unit is a benzylguanine derivative for SNAP-tag labelling, a 6-chlorohexyl derivative for HaloTag-labelling, or a benzylcytosine derivative for CLIP-tag labelling; and the fluorophore unit is from the xanthene-type series, the cyanine series or the polymethine series.

15. A compound according to claim 1, wherein the compound is selected from (i) a compound having one of the formulae (I-1) to (I-8), (ii) a compound having a formula (I-2), (I-3), or (I-4), or (iii) a compound having the formula (I-9).

16. A compound according to claim 1, wherein the fluorophore unit is from the xanthene-type series and has the formula XL-1 or XL-2: 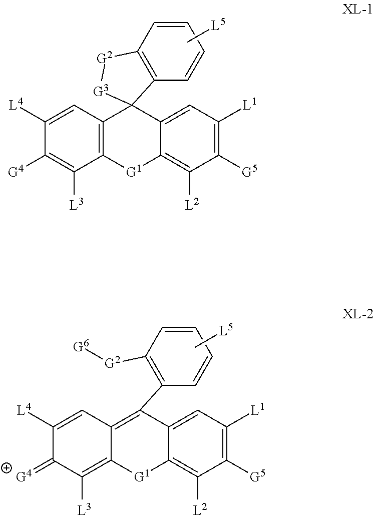
wherein L¹, L², L³ or L4 are independently selected from —H, -Hal, —NO₂, —SO₃H, —C₁₋₄-alkyl, —CN, —OMe, —OCF₃, and an —C₁₋₈-alkylene-group that connects to G⁴ and/or G⁵ to form fused rings;
zero, one, two, three, or four substituents L⁵ can be attached at any free position on the indicated benzene ring, and are independently selected from —C₁₋₄-alkyl, —O—C₁₋₄-alkyl, —CO₂H, -Hal, —C(O)—C₁₋₄-alkyl, —C(O)—O—C₁₋₄-alkyl, —C(O)—N(—C₁₋₄-alkyl)₂, —C(O)—NH(—C₁₋₄-alkyl), —SO₃H, —S(O)₂—N(—C₁₋₄-alkyl)₂, and —S(O)₂—NH(—C₁₋₄-alkyl);
G¹ is selected from —O—, —S—, —Se—, —C(—C₁₋₄-alkyl)₂-, —Si(—C₁₋₄-alkyl)₂-, —C(O)—, —CF₂—, —N(—C₁₋₄-alkyl)-, and —N(-phenyl);
G² is selected from —C(O)—, —S(O)₂—, —P(O)₂—, —CH₂—, and —CH(CH₃)—;
G³ is selected from —O—, —N(—C₁₋₄-alkyl)-, —NS(O)₂(—C₁₋₄-alkyl)-, —NS(O)₂(N(—C₁₋₄-alkyl)₂)-, —NS(O)₂(NH(—C₁₋₄-alkyl))-, and —N(C≡N)—;
G⁴ and -G⁵ are independently selected from —O—H, —O—C₁₋₄-alkyl, —NH₂, —NH—C₁₋₄-alkyl, —N(—C₁₋₄-alkyl)₂, —N(—C₁₋₆-alkylene-) (e.g. —N-azetidinyl or —N-pyrrolidinyl), —N—C(O)—C₁₋₄-alkyl, noting that (i) the =G⁴ group in XL-2 may either be a tautomer of -G⁴ in XL-1 (e.g. ═O instead of —OH) or may bear an additional positive charge (e.g. =N+Me₂ instead of —NMe₂), and that (ii) when L¹, L², L³ or L4 are alkylene groups, each (G⁴ or G⁵)-alkylene bond replaces one (G⁴ or G⁵)-H or (G⁴ or G⁵)-C₁₋₄-alkyl bond in the above definition;
G⁶ in compounds that can undergo an open-closed equilibrium is either protonated -G³-H, or unprotonated -G³-: with a lone pair as indicated (e.g. —O—); but G⁶ in compounds that are exclusively in the open form can also be -G³-C₁₋₆-alkyl or —N(—C₁₋₈-alkylene-) (e.g. —N— azetidinyl or —N-pyrrolidinyl);
and the fluorophore unit bears one or two connection points (typically, a substituent may act as a connection point, e.g. a carboxylic acid substituent —CO₂H may act as a connection point when derivatised to an amide by reaction with a linker-amine);
all “alkyl” or “alkylene” groups in the fluorophore unit can be selected independently, and each can be optionally substituted by 1-12 heteroatom-containing moieties, wherein the heteroatoms are selected from O, N, S, Se, Si, Hal, B and P, with preferable heteroatom-containing moieties being sulfonic acid or phosphonic acid groups (—SO₃H, —PO₃H₂);
any C—H in the fluorophore unit may be substituted by a C-D (deuteration), preferably at alkyl or alkylene groups of substituents G⁴, G⁵, L¹, L², L³ and/or L⁴, more preferably at G⁴ and G⁵.

17. A compound according to claim 1, wherein the fluorophore unit is from the cyanine-type series and has the formula XL-3: 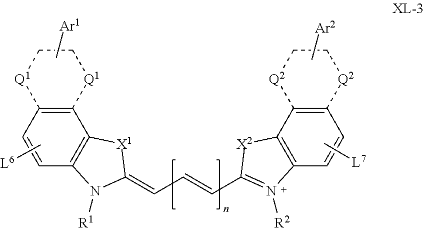
wherein
n=1-3;
X¹=O, S, or C(Me)₂;
X²=O, S, C(Me)₂, or C(Me)(R³);
R¹=Me, —C₁₋₄-alkyl, or —(CH₂)₁₋₅CO₂H;
R²=Me, —C₁₋₄-alkyl, or —(CH₂)₁₋₅CO₂H;
R³ is independently —(CH₂)₁₋₅CO₂H;
the rings Ar¹ and/or Ar² can be independently chosen to be benzene rings that annelate the indole-type phenyl ring as indicated, or not to be present (i.e. Q¹ and/or Q²=H);
zero, one, or several substituents L⁶ and/or L⁷ can be attached at any free position on the indicated benzene ring or the annelated Ar¹/Ar² ring if present, and are independently selected from —C₁₋₄-alkyl, —O—C₁₋₄-alkyl, —CO₂H, -Hal, —C(O)—C₁₋₄-alkyl, —C(O)—O—C₁₋₄-alkyl, —C(O)—N(—C₁₋₄-alkyl)₂, —C(O)—NH(—C₁₋₄-alkyl), —SO₃H, —S(O)₂—N(—C₁₋₄-alkyl)₂, and —S(O)₂—NH(—C₁₋₄-alkyl);
all “alkyl” groups can be selected independently, and each can be optionally substituted by 1-12 heteroatom-containing moieties, wherein the heteroatoms are selected from O, N, S, Se, Si, Hal, B or P, with preferable heteroatom-containing moieties being selected from sulfonic acid or phosphonic acid groups (—SO₃H, —PO₃H₂);
any C—H in the fluorophore unit may be substituted by a C-D (deuteration);
and the fluorophore unit bears one or two connection points, such as but not limited to —CO₂H, —NH₂, —C≡CH, and —N₃, independently chosen, that are required for assembling a compound of the invention (a substituent may act as a connection point, e.g. a carboxylic acid substituent —CO₂H may act as a connection point when derivatised to an amide by reaction with a linker-amine).

18. A compound according to claim 1, wherein the fluorophore is a blinking fluorophore, which preferably blinks spontaneously, or may blink extrinsically during an assay as a result of a photoreaction or a reaction with an additive such as a thiol during imaging.

19. A compound according to claim 1, wherein the fluorescence of the fluorophore is unmasked after a covalent reaction including a photochemical reaction or enzymatic reaction, or after a non-covalent association including intercalation between DNA bases or binding to nucleic acids in the minor groove, or after complexation such as of a metal cation.

20. A compound according to claim 1, wherein the fluorophore is a cyanine-type, coumarin-type, BODIPY-type, xanthene-type (including fluorescein-type, rhodol-type, and rhodamine-type), or phenoxazine-type fluorophore.

21. A compound according to claim 1, wherein the fluorophore is Cy3, Cy3.5, Cy3B, Cy5, AlexaFluor 555, AlexaFluor 647, Cy5.5, Cy7, Cy7.5, FNIR-tag, ICG, IRDye 8000W, SNIR1, IRDye 700, IRDye 78, DiO, Dil, DiO, DiR, MeOFlav7, JuloFlav7, 4-methylumbelliferone, AlexaFluor 350, AlexaFluor430, pacific blue, Star 440 SXP, Atto 425, Coumarin 153, BODIPY FL, BODIPY 507/545, BODIPY TR, BDP R6G, BODIPY 558/568, BDP 581/591, BOPHY, C11-BODIPY, BDP 650/665, fluorescein, rhodamine 123, rhodol, carbofluorescein, carborhodamine, carborhodol, ketorhodamine, ATTO 647N, JF585, silarhodamine, HMSiR, HM-DS655, SiP650, SiR 700, 680SiR, SiR680, HMSiR_{indol}, HMSiRⱼᵤₗₒₗ, HMSiR_{THQ}, Yale676_{sb}, phosphorhodamine, Nebraska Red 700, HEtetTFER, calcein, Fluo-4, oregon green, tetrachlorofluorescein, Nebraska Red rhodol, Rhodamine 110, Rhodamine 6G, Rhodamine B, tetramethylrhodamine (TMR or TAMRA), JF526, Alexa Fluor 488, Alexa Fluor 546, Alexa Fluor 594, DyLight488, JF549, Sulforhodamine 101, AlexaFluor 532, JFX549, Atto 647, Atto 488, CF 488, Rhodamine 800, Atto 550, HMJF526, HMRG, HMAcRG, JF593, sulfone-rhodamine, Atto 520, JF585, Atto 610, CP550, SiP650, TMDHS, naphthofluorescein, AlexaFluor 568, Atto 594, AlexaFluor 633, Atto 655, Atto 680, resorufin, brilliant cresyl blue, Nile blue, Nile red, methylene blue, azure B, azure A, Hoechst 33342, AlexaFluor 405, Cascade Blue, Fura-1, Fura-2, calcein AM, Fura-2-acetoxymethyl ester, Fluo-4 AM, AlexaFluor 594, Texas Red, or MaP555.

22. A method of fluorescence imaging, wherein a compound according to claim 1 is labelled onto a target that is a biomolecule; and optionally the labelled object is then applied to a biological system, or additional components are added to the labelled object; and the fluorescence emitted by the compound of claim 1 under excitation illumination is detected.

23. A method of fluorescence imaging, wherein a compound of claim 1 is introduced to a biological system so that the compound labels a target, optionally by reaction with a self-labelling protein, or optionally by bioorthogonal ligation chemistry, and the fluorescence emitted by the compound according to claim 1 under excitation illumination is detected.

24. A method of fluorescence imaging, wherein multiple compounds according to claim 1, which are the same or different, are used to label multiple targets, and fluorescence emitted by one or more of the multiple compounds according to claim 1 is detected, wherein optionally the fluorescence can be generated by direct photoexcitation of the one or more of the multiple compounds according to claim 1, or fluorescence is detected that is generated by fluorescence resonance energy transfer from a donor fluorophore that may or may not be a compound according to claim 1 to a compound according to claim 1, or else fluorescence is detected that is generated by fluorescence resonance energy transfer from a compound according to claim 1 to another fluorophore that may or may not be a compound according to claim 1.

25. A method of fluorescence imaging, wherein a compound according to claim 1 is labelled onto a target by covalent reaction or by noncovalent association, and is either imaged directly or the labelled target is brought into contact with additional assay components before imaging, wherein the imaging is done by photoexciting the compound according to claim 1 and detecting fluorescence which is emitted by fluorescence intensity imaging, fluorescence spectrum imaging, fluorescence lifetime imaging (FLIM), confocal or TIRF or superresolution (e.g. STED, MinSTED, PALM, dSTORM) imaging of fluorescence intensity; single-molecule FRET and/or multicolour FRET assays, single-particle tracking assays, calcium imaging assays, membrane potential imaging, or membrane fluidity or viscosity imaging.

26. A method of fluorescence imaging, wherein a compound according to claim 1 is photoexcited and the fluorescence which is emitted is detected by imaging, wherein the imaging is preferably fluorescence intensity imaging, fluorescence spectrum imaging, fluorescence lifetime imaging (FLIM), confocal or TIRF or superresolution imaging of fluorescence intensity; single-molecule FRET and/or multicolour FRET assays, single-particle tracking assays, calcium imaging assays, membrane potential imaging, or membrane fluidity or viscosity imaging.

27. A method of detecting a cellular process or protein or structure by fluorescence, the method comprising (i) administering to a cell, cell culture, or organism, a biomolecule such as a protein or an antibody that is labelled with a compound according to claim 1, and (ii) detecting that biomolecule in the cell, cell culture, or organism, by fluorescence which is emitted by the compound according to claim 1.

28. A method for modifying the photophysical properties of a fluorophore, the method comprising covalently linking from one to four azoaryl units to the fluorophore, wherein the nature of the covalent linkage and/or the nature of the azoaryl species is optionally varied in order to find a fluorophore-azoaryl compound structure with an optimal set of photophysical properties.
